(19) United States
(12) Patent Application Publication
(10) Pub. No.: US 2010/0298289 A1
(43) Pub. Date: Nov. 25, 2010

(54) HETEROBICYCLIC COMPOUNDS AS HISTAMINE H4-RECEPTOR ANTAGONISTS

(75) Inventors: Gilles Raphy, Brussels (BE); Cecile Pegurier, Brussels (BE); Hans Meissner, Brussels (BE); Roland Knight, Brussels (BE); David Alan Owen, Brussels (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brussels (BE)

(21) Appl. No.: 12/682,093

(22) PCT Filed: Oct. 7, 2008
(86) PCT No.: PCT/EP2008/063413

§ 371 Date: Jul. 20, 2010

(30) Foreign Application Priority Data
Oct. 9, 2007 (GB) 0719651.2
Oct. 26, 2007 (EP) 07020963.0

(51) Int. Cl. A61K 31/397; C07D 403/04; A61K 31/497; A61K 31/517; A61K 31/551; C07D 487/04; A61P 11/00; A61P 37/08; A61P 31/12; A61P 17/06; A61P 15/10; A61P 13/10; A61P 9/00; A61P 19/02

(52) U.S. Cl. 514/210.21; 544/253; 514/252.16; 514/258.1; 544/292; 514/266.22; 540/543; 514/221; 544/284; 514/252.11

(57) ABSTRACT

The present invention concerns heterobicyclic compounds of formula (I) processes for preparing them, pharmaceutical compositions containing them and their use as pharmaceuticals.

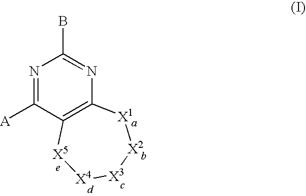

The present invention concerns novel bicyclic and heterobicyclic derivatives, processes for preparing them, pharmaceutical compositions containing them and their use as pharmaceuticals.

### BACKGROUND

To date a number of inflammatory actions of the H₄-receptor have been described: in vitro actions, calcium mobilisation and chemotaxis of murine mast cells (Hofstra et al. 2003) and eosinophils (Buckland et al., 2003; Ling et al., 2004), upregulation of adhesion molecules, CD11b/CD18 (Mac1) and CD54 on eosinophils (Buckland et al. 2003; Ling et al. 2004) and reduction in pro-inflammatory cytokine profiles following TLR ligand stimulation of dendritic cells (Dunford et al. 2006); in vivo actions, histamine-induced mast cell recruitment (Thurmond et al., 2004), neutrophil infiltration in a mouse zymosan-induced peritonitis model (Thurmond et al. 2004) and zymosan-induced neutrophilia to the pleural cavity (Takeshita et al. 2003), eosinophil recruitment (Dunford et al. 2006; Douglas et al., 2006) and mediating itch/puritis (Bell et al. 2004).

On this basis histamine H₄-receptor antagonists and inverse agonists may be used for the prophylaxis and treatment of different kind of diseases and disorders such as: respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

It has now surprisingly been found that some novel bicyclic and heterobicyclic derivatives demonstrate therapeutic properties in this field.

In one aspect, the invention provides a compound having formula I or pharmaceutically acceptable salts thereof or stereoisomeric forms thereof, and the geometrical isomers, enantiomers, diastereoisomers, and pharmaceutically acceptable salts thereof

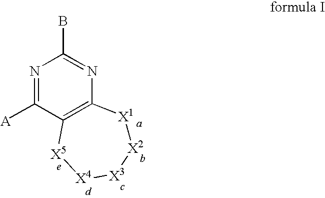

* represents the point of attachment to the rest of the molecule wherein:

B is H, NH₂, cyclopropyl, C₁₋₃ alkyl optionally substituted by cyclopropyl, NRR′;

X¹ is C(R¹)(R²), O, S, SO₂, CO or NR³;

X² is C(R⁴)(R⁵), O, S, SO₂, CO or NR⁶;

X³ is C(R⁷)(R⁸), O, S, SO₂, CO or NR⁹;

X⁴ is C(R¹⁰)(R¹¹), O, S, SO₂, CO or NR¹²;

X⁵ is C(R¹³)(R¹⁴), O, S, SO₂, CO or NR¹⁵;

a is 0 or 1;

b is 0 or 1;

c is 0 or 1;

d is 0 or 1;

e is 0 or 1;

with the proviso that a+b+c+d+e=3 or 4 or 5;

R is H, C₁₋₃ alkyl;

R′ is C₁₋₃ alkyl;

R¹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;

R² is H; or is C₁₋₃ alkyl; or can form with R¹ a C₃₋₇ cycloalkyl which is spiro-fused to the cycle (formed by X¹-X⁵); or R² can form a methylene bridge with R⁸, R¹¹ or R¹⁴;

R³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, ON, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);

R⁴ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;

R⁵ is H; or is C₁₋₃ alkyl; or can form with R⁴ a C₃₋₇ cycloalkyl which is spiro-fused to the cycle (formed by X¹-X⁵); or R⁵ can form a methylene bridge with R⁸, R¹¹ or R¹⁴;

R⁶ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, ON, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);

R⁷ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;

R⁸ is H; or is C₁₋₃ alkyl; or can form with R⁷ a C₃₋₇ cycloalkyl which is spiro-fused to the cycle (formed by X¹-X⁵); or R⁸ can form a methylene bridge with R¹¹ or R¹⁴;

R⁹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);

R¹⁰ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;

R¹¹ is H; or is C₁₋₃ alkyl; or can form with R10 a C₃₋₇ cycloalkyl which is spiro-fused to the cycle (formed by X¹-X⁵); or R¹¹ can form a methylene bridge with R¹⁴;

R¹² is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);

R¹³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;

R¹⁴ is H; or is C₁₋₃ alkyl; or can form with R¹³ a C₃₋₇ cycloalkyl which is spiro-fused to the cycle (formed by X¹-X⁵);

R¹⁵ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);

A is a group of formula II

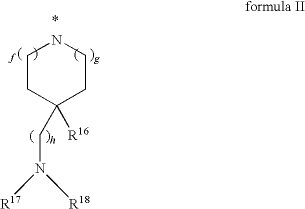

wherein

f is 0 or 1;
g is 0, 1 or 2;
h is 0 or 1;
R¹⁶ is hydrogen or unsubstituted C₁₋₃ alkyl;
R¹⁷ is hydrogen or unsubstituted C₁₋₃ alkyl;
R¹⁸ is hydrogen or unsubstituted C₁₋₃ alkyl;
or A is group of formula III

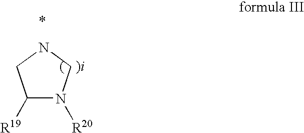

wherein

i is 2, 3;
R¹⁹ is hydrogen or unsubstituted C₁₋₃ alkyl;
R²⁰ is hydrogen or unsubstituted C₁₋₃ alkyl;
or A is a group of formula IV

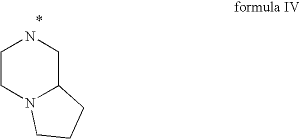

or A is a group of formula V

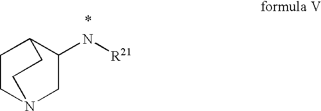

wherein

R²¹ is hydrogen or unsubstituted C₁₋₃ alkyl;
or A is a group of formula VI

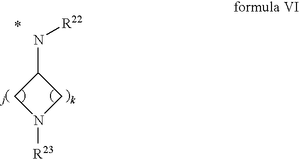

wherein
R²² is hydrogen or unsubstituted C₁₋₃ alkyl;
R²³ is hydrogen or unsubstituted C₁₋₃ alkyl;
j is 1 or 2;
k is 1 or 2;
or A is a group of formula VII

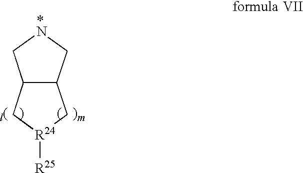

wherein

l is 1, 2 or 3;
m is 0, 1 or 2;
with the proviso that l+m=2 or 3;
R²⁴ is a CH group or N;
R²⁵ is hydrogen or unsubstituted C₁₋₃ alkyl group or is NH₂;
or A is a group of formula VIII

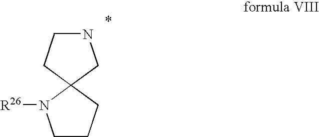

wherein

R²⁶ is hydrogen or is unsubstituted C₁₋₃ alkyl group;
or A is a group of formula IX

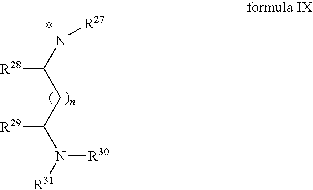

wherein

n is 0, 1 or 2;
R²⁷ is hydrogen or is unsubstituted C₁₋₃ alkyl group;
R²⁸ is hydrogen or is unsubstituted C₁₋₃ alkyl group;
R²⁹ is hydrogen or is unsubstituted C₁₋₃ alkyl group;
R³⁰ is hydrogen or is unsubstituted C₁₋₃ alkyl group;
R³¹ is hydrogen or is unsubstituted C₁₋₃ alkyl group.

The term “cycloalkyl”, as used herein, refers to a monovalent or divalent group of 3 to 6 carbon atoms, derived from a saturated cyclic hydrocarbon.

The term “cyclopropyl”, as used herein, refers to a cycloalkyl, as described above, containing 3 carbon atoms.

The term “alkyl”, as used herein, refers to saturated, monovalent or divalent hydrocarbon radicals having linear or branched moieties and containing 1-6 carbon atoms.

The term “methylene”, as used herein, refers to a group of formula —CH₂—.

The term “halogen”, as used herein, refers to an atom of chlorine, bromine, fluorine, iodine.

The term “alkoxy”, as used herein, refers to a group of formula —OR^{a} wherein R^{a} is an alkyl as defined above, containing 1 to 4 carbon atoms. C₁₋₄ alkoxy can be optionally substituted by OH or OMe.

The term “C₁₋₄ alkoxymethyl” as used herein, refers to a group of formula —CH₂—O—R, wherein, R is an alkyl group of 1 to 4 carbons as defined above.

The term “alkenyl”, as used herein refers to monovalent or divalent hydrocarbon radicals having 2 to 6 carbon atoms, derived from a saturated alkyl having at least a double bond. C₂₋₆ alkenyl groups can be in Z or E configuration.

The term “cycloalkenyl”, as used herein, refers to a monovalent or divalent group of 5 to 7 carbon atoms, derived from a saturated cycloalkyl having one double bond. Cycloalkenyl groups can be monocyclic or polycyclic.

The term “heterocycloalkyl”, as used herein refers to a monovalent or divalent group of 3 to 10 carbon atoms, derived from a saturated cyclic hydrocarbon, containing at least one heteroatom selected from O or N or S or combinations of at least two thereof, interrupting the carbocyclic ring structure. The heterocyclic ring can be interrupted by —C═O. The S heteroatom can be oxidized. Heterocycloalkyls can be optionally substituted by C₁₋₃ alkyl, halogen, C₁₋₃ alkoxy.

The term “aryl” as used herein, refers to an organic moiety derived from an aromatic hydrocarbon consisting of a ring or multiple rings, containing 6 to 10 carbon atoms by removal of one hydrogen atom, which can optionally be substituted by one or more groups selected from C₁₋₃ alkyl, halogen, C₁₋₃ alkoxy, CF₃, OCHF₂, OCF₃.

The term “aryloxy” as used herein, refers to a group of formula —OR^{b} wherein R^{b} is an aryl as defined above. Aryloxy groups can be optionally substituted by C₁₋₃ alkyl, halogen, CF₃, C₁₋₃ alkoxy, OCHF₂, OCF₃.

The term “heteroaryl”, as used herein refers to an aryl ring, as described above, containing at least one heteroatom selected from O or N or S or combinations of at least two thereof, interrupting the carbocyclic ring structure. The heteroaryl ring can be interrupted by —C═O. The S heteroatom can be oxidized. Heteroaryls can optionally be substituted by one or more groups selected from C₁₋₃ alkyl, halogen, C₁₋₃ alkoxy, CF₃, CN, OCHF₂, OCF₃. In one embodiment heteroaryls can be optionally substituted by one or more groups selected from C₁₋₃ alkyl, halogen, C₁₋₃ alkoxy, CF₃, OCHF₂, OCF₃.

The term “heteroaryloxy” as used herein, refers to a group of formula —OR^{c} wherein R^{c} is an heteroaryl as defined above. The heteroaryloxy ring can be optionally substituted by C₁₋₃ alkyl, halogen, CF₃, C₁₋₃ alkoxy, OCHF₂, OCF₃.

The term “heteroarylmethyloxy” as used herein, refers to a group of formula —OCH₂R^{d}, wherein R^{d} is a heteroaryl as defined above. Heteroarylmethyloxy rings can be optionally substituted by C₁₋₃ alkyl, halogen, CF₃, C₁₋₃ alkoxy, OCHF₂, OCF₃.

The term “benzyloxy” as used herein, refers to a group of formula —OR^{e}, wherein R^{e} is a phenyl group. The benzloxy ring can be optionally substituted by C₁₋₃ alkyl, halogen, CF₃, C₁₋₃ alkoxy, OCHF₂, OCF₃.

The term “aryl C₁₋₂ alkyl”, as used herein refers to a group of formula —CH₂-aryl or —CH₂—CH₂-aryl, where aryl is defined as above.

The term “heteroaryl C₁₋₂ alkyl”, as used herein refers to a group of formula —CH₂-heteroaryl or —CH₂—CH₂-heteroaryl, where heteroaryl is defined as above.

In one embodiment of the invention usually B is H, NH₂, cyclopropyl, C₁₋₃ alkyl optionally substituted by cyclopropyl, NRR′. In another embodiment of the invention B is H, NH₂ or C₁₋₃ alkyl, typically methyl. In a preferred embodiment of the invention B is NH₂.

In one embodiment of the invention usually X¹ is C(R¹)(R²), O, S, SO₂, CO or NR³. In a preferred embodiment of the invention X¹ is C(R¹)(R²). In another preferred embodiment of the invention X¹ is CH₂. In a further preferred embodiment of the invention X¹ is C(CH₃)(CH₃).

In one embodiment of the invention usually X² is C(R⁴)(R⁵), O, S, SO₂, CO or NR⁶. In one preferred embodiment of the invention X² is C(R⁴)(R⁵) or NR⁶. In another preferred embodiment of the invention X² is C(R⁴)(R⁵). In one preferred embodiment of the invention X² is CH₂, C(C₆H₅)(H), C(3-Cl—C₆H₅)(H), C(CH₃)(CH₃), C(2-Cl—C₆H₅)(H), C(4-F—C₆H₅)(H), C(2-NC₅H₄)(H), C(5-Cl-2SC₄H₂)(H), C(CH₂CH(CH₃)₂)(H), C(CH(CH₃)₂)(H), C(CH₂(CH₂)₃CH₂) (spiro-fused), NC(O)CH₃, N-(5-CN)pyridin-2-yl, N-(4-CF₃)pyrimidin-2-yl. In another preferred embodiment of the invention X² is CH₂, C(C₆H₅)(H), C(3-Cl—C₆H₅)(H), C(CH₃)(CH₃), C(2-Cl—C₆H₅)(H), C(4-F—C₆H₅)(H), C(2-NC₅H₄)(H), C(5-Cl-2SC₄H₂)(H). In a more preferred embodiment of the invention X² is C(CH₃)(CH₃), C(5-Cl-2SC₄H₂)(H). In a further more preferred embodiment of the invention X² is C(CH₂CH(CH₃)₂)(H), C(CH(CH₃)₂)(H), C(CH₂(CH₂)₃CH₂) (spiro-fused).

In one embodiment of the invention usually X³ is C(R⁷)(R⁸), O, S, SO₂, CO or NR⁹. In a preferred embodiment of the invention X³ is C(R⁷)(R⁸). In another preferred embodiment of the invention X³ is CH₂. In a further preferred embodiment of the invention X³ is C(CH₃)(CH₃).

In one embodiment of the invention usually X⁴ is C(R¹⁰)(R¹¹), O, S, SO₂, CO or NR¹². In a preferred embodiment of the invention X⁴ is C(R¹⁰)(R¹¹). In another preferred embodiment of the invention X⁴ is CH₂, C(5-Cl-2SC₄H₂)(H).

In one embodiment of the invention usually X⁵ is C(R¹³)(R¹⁴) O, S, SO₂, CO or NR¹⁵. In a preferred embodiment of the invention X⁵ is C(R¹³)(R¹⁴). In another preferred embodiment of the invention X⁵ is CH₂.

In one embodiment of the invention usually a is 0 or 1. In a preferred embodiment of the invention a is 1.

In one embodiment of the invention usually b is 0 or 1. In a preferred embodiment of the invention b is 1.

In one embodiment of the invention usually c is 0 or 1. In a preferred embodiment of the invention c is 1.

In one embodiment of the invention usually d is 0 or 1. In a preferred embodiment of the invention d is 1. In another preferred embodiment of the invention d is 0.

In one embodiment of the invention usually e is 0 or 1. In a preferred embodiment of the invention e is 0.

With the proviso that a+b+c+d+e=3 or 4 or 5.

In one embodiment of the invention usually R¹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃. In a preferred embodiment of the invention R¹ is hydrogen. In a further preferred embodiment of the invention R¹ is CH₃.

In one embodiment of the invention usually R² is H; or is C₁₋₃ alkyl; or can form with R¹ a C₃₋₇ cycloalkyl which is spiro-fused to the cycle (formed by X¹-X⁵); or R² can form a methylene bridge with R⁵, R⁸, R¹¹ or R¹⁴. In a preferred embodiment of the invention R² is hydrogen. In a further preferred embodiment of the invention R² is CH₃.

In one embodiment of the invention usually R³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl). In another embodiment of the invention R³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl).

In one embodiment of the invention usually R⁴ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃. In a preferred embodiment of the invention R⁴ is hydrogen, phenyl, 3-chlorophenyl, 2-chlorophenyl, 4-fluorophenyl, methyl, 2-pyridine, 2-chlorothiophene. In a more preferred embodiment R⁴ is methyl, 2-chlorothiophene. In a further more preferred embodiment R⁴ is isobutyl, isopropyl.

In one embodiment of the invention usually R⁵ is H; or is C₁₋₃ alkyl; or can form with R⁴ a C₃₋₇ cycloalkyl which is spiro-fused to the cycle (formed by X¹-X⁵); or R⁵ can form a methylene bridge with R⁸, R¹¹ or R¹⁴. In a preferred embodiment R⁵ is hydrogen, methyl.

In a more preferred embodiment R⁴ and R⁵ join to form a cyclohexyl which is spiro-fused to the cycle (formed by X¹-X⁵).

In one embodiment of the invention usually R⁶ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl). In another embodiment of the invention R⁶ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl). In a preferred embodiment of the invention R⁶ is heteroaryl optionally substituted by CF₃, CN; or is —CO(C₁₋₆ alkyl). In a preferred embodiment of the invention R⁶ is acetyl, (4-trifluoromethyl)pyrimidin-2-yl, (5-cyano)pyridin-2-yl.

In one embodiment of the invention usually R⁷ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃. In another embodiment of the invention R⁷ is phenyl, hydrogen. In a further embodiment of the invention R⁷ is methyl. In a preferred embodiment R⁷ is hydrogen.

In one embodiment of the invention usually R⁸ is H; or is C₁₋₃ alkyl; or can form with R⁷ a C₃₋₇ cycloalkyl which is spiro-fused to the cycle (formed by X¹-X⁵); or R⁸, can form a methylene bridge with R¹¹ or R¹⁴. In another embodiment of the invention R⁸ is H; or is C₁₋₃ alkyl, typically methyl. In a preferred embodiment R⁸ is hydrogen.

In one embodiment of the invention usually R⁹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl). In another embodiment of the invention R⁹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl).

In one embodiment of the invention usually R¹⁰ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃. In a preferred embodiment R¹⁰ is hydrogen, 2-chlorothiophene.

In one embodiment of the invention usually R¹¹ is H; or is C₁₋₃ alkyl; or can form with R10 a C₃₋₇ cycloalkyl which is spiro-fused to the cycle (formed by X¹-X⁵); or R¹¹ can form a methylene bridge with R¹⁴. In a preferred embodiment R¹¹ is hydrogen.

In one embodiment of the invention usually R¹² is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl). In another embodiment of the invention R¹² is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl).

In one embodiment of the invention usually R¹³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃. In another embodiment R¹³ is hydrogen.

In one embodiment of the invention usually R¹⁴ is H; or is C₁₋₃ alkyl; or can form with R¹³ a C₃₋₇ cycloalkyl which is spiro-fused to the cycle (formed by X¹-X⁵). In another embodiment R¹⁴ is hydrogen.

In one embodiment of the invention usually R¹⁵ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy; or is C₁₋₆ alkyl, CF₃, OCHF₂, OCF₃; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl). In one embodiment of the invention usually R¹⁵ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy; or is C₁₋₆ alkyl, CF₃, OCHF₂, OCF₃; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl).

In one embodiment of the invention usually A is a group of formula II wherein f is 0 or 1; g is 0, 1 or 2; h is 0 or 1; R¹⁶ is hydrogen or unsubstituted C₁₋₃ alkyl; R¹⁷ is hydrogen or unsubstituted C₁₋₃ alkyl; R¹⁸ is hydrogen or unsubstituted C₁₋₃ alkyl. In a preferred embodiment of the invention f is 0, 1; g is 0, 1; h is 0; R¹⁶ is hydrogen; R¹⁷ is hydrogen; R¹⁸ is hydrogen. In a more preferred embodiment f is 0; g is 1; h is 0; R¹⁶ is hydrogen; R¹⁷ is hydrogen; R¹⁸ is hydrogen. In another more preferred embodiment f is 0; g is 1; h is 0; R¹⁶ is hydrogen; R¹⁷ is hydrogen; R¹⁸ is methyl. In another more preferred embodiment f is 0; g is 0; h is 0; R¹⁶ is hydrogen; R¹⁷ is hydrogen; R¹⁸ is hydrogen or methyl.

In another embodiment of the invention usually A is a group of formula III wherein i is 2, 3; R¹⁹ is hydrogen or unsubstituted C₁₋₃ alkyl; R²⁰ is hydrogen or unsubstituted C₁₋₃ alkyl. In a preferred embodiment of the invention i is 2, 3; R¹⁹ is hydrogen, methyl; R²⁰ is hydrogen, methyl.

In another embodiment of the invention usually A is a group of formula IV.

In another embodiment of the invention usually A is a group of formula V wherein R²¹ is hydrogen or unsubstituted C₁₋₃ alkyl.

In another embodiment of the invention usually A is a group of formula VI wherein R²² is hydrogen or unsubstituted C₁₋₃ alkyl; R²³ is hydrogen or unsubstituted C₁₋₃ alkyl; j is 1 or 2; k is 1 or 2. In a preferred embodiment of the invention R²² is hydrogen; R²³ is hydrogen; j is 1 or 2; k is 2.

In another embodiment of the invention usually A is a group of formula VII wherein l is 1, 2 or 3; m is 0, 1 or 2; R²⁴ is a CH group or N; R²⁵ is hydrogen or unsubstituted C₁₋₃ alkyl group or is NH₂; with the proviso that l+m=2 or 3, and preferably l+m=3. In one embodiment l+m=3. In a preferred embodiment of the invention l is 3; m is 0; R²⁴ is N; R²⁵ is hydrogen. In another preferred embodiment l is 1; m is 1; R²⁴ is N; R²⁵ is hydrogen.

In another embodiment of the invention usually A is a group of formula VIII wherein usually R²⁶ is hydrogen or is unsubstituted C₁₋₃ alkyl group. In another embodiment of the invention R²⁶ is hydrogen.

In another embodiment of the invention usually A is a group of formula IX wherein usually n is 0, 1 or 2; R²⁷ is hydrogen or is unsubstituted C₁₋₃ alkyl group; R²⁸ is hydrogen or is unsubstituted C₁₋₃ alkyl group; R²⁹ is hydrogen or is unsubstituted C₁₋₃ alkyl group; R³⁰ is hydrogen or is unsubstituted C₁₋₃ alkyl group; R³¹ is hydrogen or is unsubstituted C₁₋₃ alkyl group. In a preferred embodiment of the invention n is 0; R²⁷ is hydrogen; R²⁸ is hydrogen; R²⁹ is hydrogen; R³⁰ is hydrogen; R³¹ is methyl.

In one embodiment of the invention B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula II wherein f is 0 or 1; g is 0, 1 or 2; h is 0 or 1; R¹⁶ is hydrogen or unsubstituted C₁₋₃ alkyl; R¹⁷ is hydrogen or unsubstituted C₁₋₃ alkyl; R¹⁸ is hydrogen or unsubstituted C₁₋₃ alkyl.

In another embodiment of the invention B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 0 or 1; and e is 0 or 1; and A is a group of formula III wherein i is 2, 3; R¹⁹ is hydrogen or unsubstituted C₁₋₃ alkyl; R²⁰ is hydrogen or unsubstituted C₁₋₃ alkyl. In one embodiment d is 1.

In another embodiment of the invention B is NH₂; and X¹ is C(R¹)(R²); and X² is NR⁶; and X³ is C(R⁷)(R⁸); and X⁴ is C(R¹⁰)(R¹¹); and a is 1; and b is 1; and c is 1; and d is 0 or 1; and e is 0; and A is a group of formula III wherein i is 2; R¹⁹ is hydrogen; R²⁰ is unsubstituted C₁₋₃ alkyl.

In another embodiment of the invention B is CH₃ or H; and X¹ is C(R¹)(R²); and X² is C(R⁴)(R⁵); and X³ is C(R⁷)(R⁸); and X⁴ is C(R¹⁰)(R¹¹); and a is 1; and b is 1; and c is 1; and d is 1; and e is 0; and A is a group of formula III wherein i is 2; R¹⁹ is hydrogen; R²⁰ is unsubstituted C₁₋₃ alkyl.

In another embodiment of the invention B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 0 or 1; and e is 0 or 1; and A is a group of formula VI wherein R²² is hydrogen or unsubstituted C₁₋₃ alkyl; R²³ is hydrogen or unsubstituted C₁₋₃ alkyl; j is 1 or 2, k is 2.

In another embodiment of the invention B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula VII wherein l is 1, 2 or 3; m is 0, 1 or 2; R²⁴ is a CH group or N; R²⁵ is hydrogen or unsubstituted C₁₋₃ alkyl group or is NH₂; with the proviso that l+m=2 or 3. In one embodiment l+m=3.

In another embodiment of the invention B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula VIII wherein R²⁶ is hydrogen or is unsubstituted C₁₋₃ alkyl group.

In another embodiment of the invention B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula IX wherein n is 0; R²⁷ is hydrogen or is unsubstituted C₁₋₃ alkyl; R²⁸ is hydrogen or is unsubstituted C₁₋₃ alkyl; R²⁹ is hydrogen or is unsubstituted C₁₋₃ alkyl; R³⁹ is hydrogen or is unsubstituted C₁₋₃ alkyl; R³¹ is hydrogen or is unsubstituted C₁₋₃ alkyl.

In another embodiment of the invention B is NH₂; and X¹ is CH₂, C(CH₃)(CH₃) and X² is CH₂, C(C₆H₅)(H), C(3Cl—C₆H₅)(H), C(CH₃)(CH₃), C(2Cl—C₆H₅)(H), C(CH₂CH(CH₃)₂)(H), C(CH(CH₃)₂)(H), C(CH₂(CH₂)₃CH₂) (spiro-fused); and X³ is CH₂, C(C₆H₅)(H); and X⁴ is CH₂; and X⁵ is CH₂; and a is 1; and b is 1; and c is 1; and d is 1; and e is 0 or 1; and A is a group of formula II wherein f is 0; g is 0, 1; h is 0; R¹⁶ is hydrogen; R¹⁷ is hydrogen; R¹⁸ is hydrogen or methyl.

In another embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H); and X³ is CH₂, C(C₆H₅)(H); and X⁴ is CH₂; and X⁵ is CH₂; and a is 1; and b is 1; and c is 1; and d is 1; and e is 0 or 1; and A is a group of formula II wherein f is 0; g is 0, 1; h is 0; R¹⁶ is hydrogen; R¹⁷ is hydrogen; R¹⁸ is hydrogen.

In another embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H), C(4FI—C₆H₅)(H), C(2NC₅H₄)(H), C(5-Cl-2SC₄H₂)(H), C(CH₂CH(CH₃)₂)(H), C(CH(CH₃)₂)(H), C(CH₂(CH₂)₃CH₂) (spiro-fused); and X³ is CH₂; and X⁴ is CH₂; and X⁵ is CH₂; and a is 1; and b is 1; and c is 1; and d is 0 or 1; and e is 0 or 1; and A is a group of formula III wherein i is 2, 3; R¹⁹ is hydrogen, methyl; R²⁰ is methyl, hydrogen.

In another embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H), C(4FI—C₆H₅)(H), C(2NC₅H₄)(H), C(5-Cl-2SC₄H₂)(H); and X³ is CH₂; and X⁴ is CH₂; and X⁵ is CH₂; and a is 1; and b is 1; and c is 1; and d is 1; and e is 0 or 1; and A is a group of formula III wherein i is 2, 3; R¹⁹ is hydrogen, methyl; R²⁰ is methyl, hydrogen.

In another embodiment of the invention B is NH₂; and X¹ is CH₂; and X² is CH₂; and X³ is C(CH₃)(CH₃); and X⁴ is CH₂; and X⁵ is CH₂; and a is 1; and b is 1; and c is 1; and d is 1; and e is 0 or 1; and A is a group of formula III wherein i is 2, 3; R¹⁹ is hydrogen, methyl; R²⁰ is methyl, hydrogen.

In another embodiment of the invention B is NH₂; and X¹ is C(CH₃)(CH₃); and X² is CH₂; and X³ is CH₂; and X⁴ is CH₂; and X⁵ is CH₂; and a is 1; and b is 1; and c is 1; and d is 1; and e is 0 or 1; and A is a group of formula III wherein i is 2, 3; R¹⁹ is hydrogen, methyl; R²⁰ is methyl, hydrogen.

In another embodiment of the invention B is NH₂; and X¹ is CH₂; and X² is NC(O)CH₃, N-(5-CN)pyridin-2-yl, N-(4-CF₃)pyrimidin-2-yl; and X³ is CH₂; and X⁴ is CH₂; and a is 1; and b is 1; and c is 1; and d is 0 or 1; and e is 0; and A is a group of formula III wherein i is 2; R¹⁹ is hydrogen; R²⁰ is methyl.

In another embodiment of the invention B is CH₃ or H; and X¹ is CH₂; and X² is C(CH₃)₂; and X³ is CH₂; and X⁴ is CH₂; and a is 1; and b is 1; and c is 1; and d is 1; and e is 0; and A is a group of formula III wherein i is 2; R¹⁹ is hydrogen; R²⁰ is methyl.

In another embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H), C(4F—C₆H₅)(H), C(CH(CH₃)₂)(H), C(CH₂(CH₂)₃CH₂) (spiro-fused); and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; or l is 1, m is 1; R²⁴ is N; R²⁵ is hydrogen.

In another embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H); and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen.

In another embodiment of the invention B is NH₂; and X¹ is C(CH₃)(CH₃); X² is CH₂; and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen.

In another embodiment of the invention B is NH₂; and X¹ is CH₂; and X² is CH₂, C(C₆H₅)(H); and X³ is CH₂; and X⁴ is CH₂; X⁵ is CH₂; and a is 1; and b is 1; and c is 1; and d is 1; and e is 0 or 1; and A is a group of formula VIII wherein R²⁶ is hydrogen.

In a preferred embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H), C(CH₂CH(CH₃)₂)(H), C(CH(CH₃)₂)(H), C(CH₂(CH₂)₃CH₂) (spiro-fused); and X³ is CH₂; and X⁴ is CH₂; and X⁵ is CH₂; and a is 1; and b is 1; and c is 1; and d is 1; and e is 0 or 1; and A is a group of formula II wherein f is 0, 1; g is 0, 1; h is 0; R¹⁶ is hydrogen; R¹⁷ is hydrogen; R¹⁸ is hydrogen or methyl. In a further preferred embodiment of the invention B is NH₂; and X¹ is CH₂; and X² is CH₂, C(C₆H₅)(H), C(3Cl—C₆H₅)(H), C(CH₃)(CH₃), C(2Cl—C₆H₅)(H); and X³ is CH₂; and X⁴ is CH₂; and X⁵ is CH₂; and a is 1; and b is 1; and c is 1; and d is 1; and e is 0 or 1; and A is a group of formula II wherein f is 0, 1; g is 0, 1; h is 0; R¹⁶ is hydrogen; R¹⁷ is hydrogen; R¹⁸ is hydrogen.

In another preferred embodiment of the invention B is NH₂; and X¹ is C(CH₃)(CH₃); and X² is CH₂; and X³ is CH₂; and X⁴ is CH₂; and X⁵ is CH₂; and a is 1; and b is 1; and c is 1; and d is 1; and e is 0 or 1; and A is a group of formula II wherein f is 0; g is 0; h is 0; R¹⁶ is hydrogen; R¹⁷ is hydrogen; R¹⁸ is hydrogen.

In another preferred embodiment of the invention B is NH₂; and X¹ is CH₂; and X² is C(C₆H₅)(H), C(3Cl—C₆H₅)(H), C(CH₃)(CH₃), C(2Cl—C₆H₅)(H), C(4FI—C₆H₅)(H), C(2NC₅H₄)(H), C(5-Cl-2SC₄H₂)(H), C(CH₂CH(CH₃)₂)(H), C(CH(CH₃)₂)(H), C(CH₂(CH₂)₃CH₂) (spiro-fused); and X³ is CH₂; and X⁴ is CH₂, C(5-Cl-2SC₄H₂)(H); and X⁵ is CH₂; and a is 1; and b is 1; and c is 1; and d is 0 or 1; and e is 0 or 1; and A is a group of formula III wherein i is 2, 3; R¹⁹ is hydrogen, methyl; R²⁰ is methyl, hydrogen. In a further preferred embodiment B is NH₂; and X¹ is CH₂; and X² is C(C₆H₅)(H), C(3Cl—C₆H₅)(H), C(CH₃)(CH₃), C(2Cl—C₆H₅)(H), C(4FI—C₆H₅)(H), C(2NC₅H₄)(H), C(5-Cl-2SC₄H₂)(H); and X³ is CH₂; and X⁴ is CH₂, C(5-Cl-2SC₄H₂)(H); and X⁵ is CH₂; and a is 1; and b is 1; and c is 1; and d is 1; and e is 0 or 1; and A is a group of formula III wherein i is 2, 3; R¹⁹ is hydrogen, methyl; R²⁰ is methyl, hydrogen. In another preferred embodiment of the invention B is NH₂; and X¹ is C(CH₃)(CH₃); and X² is CH₂; and X³ is CH₂; and X⁴ is CH₂; and X⁵ is CH₂; and a is 1; and b is 1; and c is 1; and d is 1; and e is 0; and A is a group of formula III wherein i is 2, 3; R¹⁹ is hydrogen, methyl; R²⁰ is methyl, hydrogen.

In another preferred embodiment of the invention B is NH₂; and X¹ is CH₂; and X² is N-(4-CF₃)pyrimidin-2-yl; and X³ is CH₂; and X⁴ is CH₂; and a is 1; and b is 1; and c is 1; and d is 0 or 1; and e is 0; and A is a group of formula III wherein i is 2; R¹⁹ is hydrogen; R²⁰ is methyl.

In another preferred embodiment of the invention B is NH₂; and X¹ is C(CH₃)(CH₃); and X² is CH₂; and X³ is CH₂; and X⁴ is CH₂; and X⁵ is CH₂; and a is 1; and b is 1; and c is 1; and d is 1; and e is 0; and A is a group of formula VI wherein R²² is hydrogen; R²³ is hydrogen; j is 1 or 2; k is 2.

In another preferred embodiment of the invention B is NH₂; and X¹ is CH₂; and X² is C(C₆H₅)(H), C(3Cl—C₆H₅)(H), C(CH₃)(CH₃), C(2Cl—C₆H₅)(H), C(4F—C₆H₅)(H), C(CH(CH₃)₂)(H), C(CH₂(CH₂)₃CH₂) (spiro-fused); and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen. In another preferred embodiment of the invention B is NH₂; and X¹ is CH₂; and X² is C(C₆H₅)(H), C(3Cl—C₆H₅)(H), C(CH₃)(CH₃), C(2Cl—C₆H₅)(H); and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen.

In another preferred embodiment of the invention B is NH₂; and X¹ is CH₂; and X² is C(CH₃)(CH₃); and X³ is CH₂; and X⁴ is CH₂; and X⁵ is CH₂; and a is 1; and b is 1; and c is 1; and d is 1; and e is 0; and A is a group of formula VII wherein l is 1; m is 1; R²⁴ is N; R²⁵ is hydrogen.

In another preferred embodiment of the invention B is NH₂; and X¹ is CH₂; and X² is C(4F—C₆H₅)(H), and X³ is CH₂; and X⁴ is CH₂; and X⁵ is CH₂; and a is 1; and b is 1; and c is 1; and d is 1; and e is 0; and A is a group of formula IX n is 0; R²⁷ is hydrogen; R²⁸ is hydrogen; R²⁹ is hydrogen; R³⁰ is hydrogen; R³¹ is hydrogen.

In a more preferred embodiment of the invention B is NH₂; and X¹ is CH₂; and X² is C(CH₃)(CH₃), C(5-Cl-2SC₄H₂)(H), C(CH₂CH(CH₃)₂)(H), C(CH(CH₃)₂)(H), C(CH₂(CH₂)₃CH₂) (spiro-fused); and X³ is CH₂; and X⁴ is CH₂; and X⁵ is CH₂; and a is 1; and b is 1; and c is 1; and d is 1; and e is 0 or 1; and A is a group of formula III wherein i is 2, 3; R¹⁹ is hydrogen, methyl; R²⁰ is methyl, hydrogen. In a further more preferred embodiment of the invention B is NH₂; and X¹ is CH₂; and X² is C(CH₃)(CH₃), C(5-Cl-2SC₄H₂)(H); and X³ is CH₂; and X⁴ is CH₂; and X⁵ is CH₂; and a is 1; and b is 1; and c is 1; and d is 1; and e is 0 or 1; and A is a group of formula III wherein i is 2, 3; R¹⁹ is hydrogen, methyl; R²⁰ is methyl, hydrogen.

In another more preferred embodiment of the invention B is NH₂; and X¹ is C(CH₃)(CH₃); and X² is CH₂; and X³ is CH₂; and X⁴ is CH₂; and X⁵ is CH₂; and a is 1; and b is 1; and c is 1; and d is 1; and e is 0; and A is a group of formula III wherein i is 2, 3; R¹⁹ is hydrogen; R²⁰ is methyl, hydrogen.

In another more preferred embodiment of the invention B is NH₂; and X¹ is CH₂; and X² is C(CH₃)(CH₃), C(CH₂CH(CH₃)₂)(H), C(CH(CH₃)₂)(H), C(CH₂(CH₂)₃CH₂) (spiro-fused); and X³ is CH₂; and X⁴ is CH₂; and a is 1; and b is 1; and c is 1; and d is 1; and e is 0; and A is a group of formula II wherein f is 0; g is 0 or 1; h is 0; R¹⁶ is hydrogen; R¹⁷ is hydrogen; R¹⁸ is hydrogen or methyl. In another more preferred embodiment of the invention B is NH₂; and X¹ is CH₂; and X² is C(CH₃)(CH₃); and X³ is CH₂; and X⁴ is CH₂; and a is 1; and b is 1; and c is 1; and d is 1; and e is 0; and A is a group of formula II wherein f is 0; g is 1; h is 0; R¹⁶ is hydrogen; R¹⁷ is hydrogen; R¹⁸ is hydrogen.

In another more preferred embodiment of the invention B is NH₂; and X¹ is CH₂; and X² is C(CH₃)(CH₃), C(CH(CH₃)₂)(H), C(CH₂(CH₂)₃CH₂) (spiro-fused); and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen. In another more preferred embodiment of the invention B is NH₂; and X¹ is CH₂; and X² is C(CH₃)(CH₃), and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen.

In another more preferred embodiment of the invention B is NH₂; and X¹ is C(CH₃)(CH₃); and X² is CH₂; and X³ is CH₂; and X⁴ is CH₂; and X⁵ is CH₂; and a is 1; and b is 1; and c is 1; and d is 1; and e is 0 or 1; and A is a group of formula VII wherein l is 3; m is 0; R²⁴ is N; R²⁵ is hydrogen.

Preferred compounds of the invention are:
4-(4-methylpiperazin-1-yl)-7-phenyl-5,6,7,8-tetrahydroquinazolin-2-amine;
4-(3-aminoazetidin-1-yl)-7-phenyl-5,6,7,8-tetrahydroquinazolin-2-amine;
4-(3-aminopyrrolidin-1-yl)-7-phenyl-5,6,7,8-tetrahydroquinazolin-2-amine;
4-[(4aR*,7aR*)-octahydro-6H-pyrrolo[3,4-b]pyridin-6-yl]-7-phenyl-5,6,7,8-tetrahydroquinazolin-2-amine;
4-(3-methylpiperazin-1-yl)-7-phenyl-5,6,7,8-tetrahydroquinazolin-2-amine;
7-(3-chlorophenyl)-4-(4-methylpiperazin-1-yl)-5,6,7,8-tetrahydroquinazolin-2-amine;
7-(3-chlorophenyl)-4-(1,4-diazepan-1-yl)-5,6,7,8-tetrahydroquinazolin-2-amine;
7-(3-chlorophenyl)-4-[(4aR*,7aR*)-octahydro-6H-pyrrolo[3,4-b]pyridin-6-yl]-5,6,7,8-tetrahydroquinazolin-2-amine;
7,7-dimethyl-4-(4-methylpiperazin-1-yl)-5,6,7,8-tetrahydroquinazolin-2-amine;
4-(1,4-diazepan-1-yl)-7,7-dimethyl-5,6,7,8-tetrahydroquinazolin-2-amine;
4-(3-aminopyrrolidin-1-yl)-7,7-dimethyl-5,6,7,8-tetrahydroquinazolin-2-amine;
7,7-dimethyl-4-[(4aR*,7aR*)-octahydro-6H-pyrrolo[3,4-b]pyridin-6-yl]-5,6,7,8-tetrahydroquinazolin-2-amine;
4-(3-aminopyrrolidin-1-yl)-7-(3-chlorophenyl)-5,6,7,8-tetrahydroquinazolin-2-amine;
7,7-dimethyl-4-(3-methylpiperazin-1-yl)-5,6,7,8-tetrahydroquinazolin-2-amine;
7,7-dimethyl-4-piperazin-1-yl-5,6,7,8-tetrahydroquinazolin-2-amine;
7-(2-chlorophenyl)-4-(4-methylpiperazin-1-yl)-5,6,7,8-tetrahydroquinazolin-2-amine;
7-(2-chlorophenyl)-4-(1,4-diazepan-1-yl)-5,6,7,8-tetrahydroquinazolin-2-amine;
4-(3-aminopyrrolidin-1-yl)-7-(2-chlorophenyl)-5,6,7,8-tetrahydroquinazolin-2-amine;
4-(1,4-diazepan-1-yl)-7-(4-fluorophenyl)-5,6,7,8-tetrahydroquinazolin-2-amine;
7-(4-fluorophenyl)-4-(4-methylpiperazin-1-yl)-5,6,7,8-tetrahydroquinazolin-2-amine;
4-piperazin-1-yl-7-pyridin-2-yl-5,6,7,8-tetrahydroquinazolin-2-amine;
4-(4-methylpiperazin-1-yl)-7-pyridin-2-yl-5,6,7,8-tetrahydroquinazolin-2-amine;
4-(1,4-diazepan-1-yl)-7-pyridin-2-yl-5,6,7,8-tetrahydroquinazolin-2-amine;
7-(5-chloro-2-thienyl)-4-piperazin-1-yl-5,6,7,8-tetrahydroquinazolin-2-amine;
5-(5-chloro-2-thienyl)-4-piperazin-1-yl-5,6,7,8-tetrahydroquinazolin-2-amine;
5-(5-chloro-2-thienyl)-4-(4-methylpiperazin-1-yl)-5,6,7,8-tetrahydroquinazolin-2-amine;
7-(5-chloro-2-thienyl)-4-(4-methylpiperazin-1-yl)-5,6,7,8-tetrahydroquinazolin-2-amine;
5-(5-chloro-2-thienyl)-4-(1,4-diazepan-1-yl)-5,6,7,8-tetrahydroquinazolin-2-amine;
7-(5-chloro-2-thienyl)-4-(1,4-diazepan-1-yl)-5,6,7,8-tetrahydroquinazolin-2-amine.

Further preferred compounds of the invention are:
7-(4-fluorophenyl)-4-[(4aR*,7aR*)-octahydro-6H-pyrrolo[3,4-b]pyridin-6-yl]-5,6,7,8-tetrahydroquinazolin-2-amine trifluoroacetic acid salt;
7-(4-fluorophenyl)-N-4-[2-(methylamino)ethyl]-5,6,7,8-tetrahydroquinazoline-2,4-diamine;
7,7-dimethyl-N-4-[2-(methylamino)ethyl]-5,6,7,8-tetrahydroquinazoline-2,4-diamine;
4-(hexahydropyrrolo[3,4-c]pyrrol-2(1H)-yl)-7,7-dimethyl-5,6,7,8-tetrahydroquinazolin-2-amine;
7,7-dimethyl-4-[(3S)-3-methyl-1,4-diazepan-1-yl]-5,6,7,8-tetrahydroquinazolin-2-amine;
8,8-dimethyl-4-(3-methylpiperazin-1-yl)-5,6,7,8-tetrahydroquinazolin-2-amine;
4-(3-aminoazetidin-1-yl)-8,8-dimethyl-5,6,7,8-tetrahydroquinazolin-2-amine;
8,8-dimethyl-N-4-piperidin-4-yl-5,6,7,8-tetrahydroquinazoline-2,4-diamine;
8,8-dimethyl-N-4-pyrrolidin-3-yl-5,6,7,8-tetrahydroquinazoline-2,4-diamine;
8,8-dimethyl-4-[(3S)-3-methylpiperazin-1-yl]-5,6,7,8-tetrahydroquinazolin-2-amine;
6,6-dimethyl-4-(4-methylpiperazin-1-yl)-6,7-dihydro-5H-cyclopenta[d]pyrimidin-2-amine;
4-(4-methylpiperazin-1-yl)-7-[4-(trifluoromethyl)pyrimidin-2-yl]-5,6,7,8-tetrahydropyrido[3,4-d]pyrimidin-2-amine.

More preferred compounds of the invention are:
7,7-dimethyl-4-(4-methylpiperazin-1-yl)-5,6,7,8-tetrahydroquinazolin-2-amine;
4-(1,4-diazepan-1-yl)-7,7-dimethyl-5,6,7,8-tetrahydroquinazolin-2-amine;
4-(3-aminopyrrolidin-1-yl)-7,7-dimethyl-5,6,7,8-tetrahydroquinazolin-2-amine;
7,7-dimethyl-4-[(4aR*,7aR*)-octahydro-6H-pyrrolo[3,4-b]pyridin-6-yl]-5,6,7,8-tetrahydroquinazolin-2-amine;
7,7-dimethyl-4-(3-methylpiperazin-1-yl)-5,6,7,8-tetrahydroquinazolin-2-amine;
7,7-dimethyl-4-piperazin-1-yl-5,6,7,8-tetrahydroquinazolin-2-amine;
7-(5-chloro-2-thienyl)-4-piperazin-1-yl-5,6,7,8-tetrahydroquinazolin-2-amine;
7-(5-chloro-2-thienyl)-4-(1,4-diazepan-1-yl)-5,6,7,8-tetrahydroquinazolin-2-amine.

Further more preferred compounds of the invention are:
4-(3-aminoazetidin-1-yl)-7,7-dimethyl-5,6,7,8-tetrahydroquinazolin-2-amine;
7-isobutyl-4-piperazin-1-yl-5,6,7,8-tetrahydroquinazolin-2-amine;
8,8-dimethyl-4-(4-methylpiperazin-1-yl)-5,6,7,8-tetrahydroquinazolin-2-amine;
4-(1,4-diazepan-1-yl)-8,8-dimethyl-5,6,7,8-tetrahydroquinazolin-2-amine bis acetic acid salt;
7,7-dimethyl-4-[3-(methylamino)pyrrolidin-1-yl]-5,6,7,8-tetrahydroquinazolin-2-amine;
7,7-dimethyl-4-[(3S)-3-methylpiperazin-1-yl]-5,6,7,8-tetrahydroquinazolin-2-amine;
4-(1,4-diazepan-1-yl)-7-isobutyl-5,6,7,8-tetrahydroquinazolin-2-amine acetic acid salt;
7-isobutyl-4-(4-methylpiperazin-1-yl)-5,6,7,8-tetrahydroquinazolin-2-amine acetic acid salt;
7,7-dimethyl-4-[3-(methylamino)azetidin-1-yl]-5,6,7,8-tetrahydroquinazolin-2-amine;
8,8-dimethyl-4-[(4aR*,7aR*)-octahydro-6H-pyrrolo[3,4-b]pyridin-6-yl]-5,6,7,8-tetrahydroquinazolin-2-amine;
4′-(4-methylpiperazin-1-yl)-5′,8′-dihydro-6′H-spiro[cyclohexane-1,7′-quinazolin]-2′-amine;
4′-[(4aR*,7aR*)-octahydro-6H-pyrrolo[3,4-b]pyridin-6-yl]-5′,8′-dihydro-6′H-spiro[cyclohexane-1,7′-quinazolin]-2′-amine;
4′-[(3S)-3-methylpiperazin-1-yl]-5′,8′-dihydro-6′H-spiro[cyclohexane-1,7′-quinazolin]-2′-amine bis acetic acid salt;
4′-(1,4-diazepan-1-yl)-5′,8′-dihydro-6′H-spiro[cyclohexane-1,7′-quinazolin]-2′-amine bis acetic acid salt;
4′-piperazin-1-yl-5′,8′-dihydro-6′H-spiro[cyclohexane-1,7′-quinazolin]-2′-amine bis acetic acid salt;
4′-[3-(methylamino)azetidin-1-yl]-5′,8′-dihydro-6′H-spiro[cyclohexane-1,7′-quinazolin]-2′-amine bis acetate salt;
4-(3-aminopyrrolidin-1-yl)-7-isobutyl-5,6,7,8-tetrahydroquinazolin-2-amine bis acetate salt;
7-isopropyl-4-(4-methylpiperazin-1-yl)-5,6,7,8-tetrahydroquinazolin-2-amine;
7-isopropyl-4-[(3S)-3-methylpiperazin-1-yl]-5,6,7,8-tetrahydroquinazolin-2-amine bis acetic acid salt;
7-isopropyl-4-[(4aR*,7aR*)-octahydro-6H-pyrrolo[3,4-b]pyridin-6-yl]-5,6,7,8-tetrahydroquinazolin-2-amine;
7-isopropyl-4-[3-(methylamino)azetidin-1-yl]-5,6,7,8-tetrahydroquinazolin-2-amine.

Best results have been obtained with the compound tert-butyl 4-(2-amino-7,7-dimethyl-5,6,7,8-tetrahydroquinazolin-4-yl)-1,4-diazepane-1-carboxylate.

The “pharmaceutically acceptable salts” according to the invention include all therapeutically active, non-toxic acid salt forms which the compounds of formula (I) are able to form. The acid addition salt form of a compound of formula (I) that occurs in its free form as a base can be obtained by treating the free base with an appropriate acid such as an inorganic acid, for example, a hydrohalic such as hydrochloric, hydroiodic or hydrobromic, sulfuric, nitric, phosphoric and the like; or an organic acid, such as, for example, acetic, oxalic, p-bromophenylsulfonic, carbonic, benzoic, formic, propionic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, palmoic, and the like. Conversely said salt forms can be converted into the free forms by treatment with an appropriate base.

The “pharmaceutically acceptable salts” according to the invention include therapeutically active, non-toxic base salt forms which the compounds of formula I are able to form. For example the compounds of formula I containing acidic protons may be converted into their therapeutically active, non-toxic base addition salt forms, e.g. metal or amine salts, by treatment with appropriate organic and inorganic bases. Appropriate base salt forms include, for example but are not limited to, ammonium salts, alkali and alkaline earth metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like. Conversely said salt forms can be converted into the free forms by treatment with an appropriate acid.

Compounds of the formula I and their salts can be in the form of solvates, which are included within the scope of the present invention. Such solvates include for example hydrates, alcoholates and the like.

Some of the compounds of formula I and some of their intermediates have at least one stereogenic centre in their structure. This stereogenic centre may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula I or mixtures thereof (including all possible mixtures of stereoisomers).

Some of the compounds of formula I may also exist in tautomeric forms. Such forms although not explicitly indicated in the above formula are intended to be included within the scope of the present invention.

With respect to the present invention reference to a compound or compounds is intended to encompass that compound in each of its possible isomeric forms and mixtures thereof unless the particular isomeric form is referred to specifically.

Compounds according to the present invention may exist in different polymorphic forms. Although not explicitly indicated in the above formula, such forms are intended to be included within the scope of the present invention.

The invention also includes within its scope prodrug forms of the compounds of formula I and its various sub-scopes and sub-groups.

The term “prodrug” as used herein includes compound forms, which are rapidly transformed in vivo to the parent compound according to the invention, for example, by hydrolysis in blood. Prodrugs are compounds bearing groups that are removed by biotransformation prior to exhibiting their pharmacological action. Such groups include moieties that are readily cleaved in vivo, from the compound bearing it, which compound after cleavage remains or becomes pharmacologically active. Metabolically cleavable groups form a class of groups well known to practitioners in the art. The compounds bearing the metabolically cleavable groups have the advantage that they may exhibit improved bioavailability as a result of enhanced solubility and/or rate of absorption conferred upon the parent compound by virtue of the presence of the metabolically cleavable group (T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987).

It has now been found that compounds of formula I and their pharmaceutically acceptable salts are useful in a variety of pharmaceutical indications. For example, the compounds according to the invention are useful for the treatment of inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

Thus, the present invention, in a further aspect, concerns the use of a compound of formula I or a pharmaceutically acceptable salt thereof for the manufacture of a medicament for the treatment of disorders such as mentioned above.

In particular, the present invention concerns the use of a compound of formula I or a pharmaceutically acceptable salt thereof, for the manufacture of a medicament for the treatment of H₄ dependent conditions, such as inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis or autoimmune diseases including rheumatoid arthritis, multiple sclerosis.

The compounds of the invention are useful for treating conditions in which there is an influx of leukocytes in the tissues. These conditions include inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis or autoimmune diseases including rheumatoid arthritis, multiple sclerosis.

The compounds of the invention exhibit the biological activity by inhibiting the histamine binding to the H₄ receptor or on an activated H₄ receptor. Subjects in need of treatment for a H₄ dependent inflammatory disorder or inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis or autoimmune diseases including rheumatoid arthritis, multiple sclerosis, can be treated by administering to the patient an effective amount of one or more of the above-identified compounds or a pharmaceutically acceptable derivative or salt thereof in a pharmaceutically acceptable carrier or diluent to reduce formation of oxygen radicals. The active materials can be administered by any appropriate route, for example, orally, parenterally, intravenously, intradermally, subcutaneously, intramuscularly or topically, in liquid, cream, gel or solid form, via a buccal or nasal spray, or aerosol or patch.

The invention further concerns the use of the compounds of formula I for the manufacture of a medicament for therapeutic application.

In particular, the invention concerns the use of the compounds of formula I for the manufacture of a medicament useful for treating conditions in which there is likely to be a H₄ dependent inflammatory component.

The invention concerns the use of the compound of formula I for the manufacture of a medicament useful for treating inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

The invention further concerns the compounds of formula I for use as medicaments. The invention concerns the compounds of formula I for use as a medicament for inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; nociceptive pain and/or disease-induced neuropathic pain, acute pain, neuropathic pain, diabetic pain, chronic pain, muscular pain, inflammatory pain, lymphatic diseases.

The activity and properties of the active compounds, oral availability and stability in vitro or in vivo can vary significantly among the optical isomers of the disclosed compounds.

In a preferred embodiment, the active compound is administered in an enantiomerically enriched form, i.e., substantially in the form of one isomer. By the term “substantially” we understand greater or equal to 95% of the said isomer.

The present invention also concerns a method for treating H₄ dependent inflammatory conditions inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or diseases of the gastrointestinal tract such as inflammatory bowel disease, Crohn's disease, ulcerative colitis or autoimmune diseases such as rheumatoid arthritis, multiple sclerosis, atherosclerosis, skin diseases where there's an influx of inflammatory cells, cardiovascular diseases, in a mammal in need of such treatment, comprising administering a therapeutic dose of at least one compound of formula I or a pharmaceutically acceptable salt thereof to a patient.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 0.01 to 1000 mg, preferably 0.05 to 500 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The activity of the compounds of formula I or their pharmaceutically acceptable salts, as H₄ antagonists can be determined in a tritiated histamine binding assay and in a H₄ GTPγS³⁵ binding assay. The objective of this test is to evaluate the anti-H₄ potential of a compound by measuring its inhibitory effect on histamine binding to the H₄ receptor or on H₄ receptor activation. Results obtained with compounds of formula I are indicative of a strong pharmacological effect.

For treating diseases, compounds of formula I or their pharmaceutically acceptable salts, may be employed at an effective daily dosage and administered in the form of a pharmaceutical composition.

Therefore, another embodiment of the present invention concerns a pharmaceutical composition comprising an effective amount of a compound of formula I or a pharmaceutically acceptable salt thereof in combination with a pharmaceutically acceptable diluent or carrier.

To prepare a pharmaceutical composition according to the invention, one or more of the compounds of formula I or a pharmaceutically acceptable salt thereof, is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, or parenteral.

Pharmaceutical compositions comprising compounds according to the invention can, for example, be administered orally or parenterally, i.e., intravenously, intramuscularly, subcutaneously, transdermally, intrathecally or by inhalation.

Pharmaceutical compositions suitable for oral administration can be solids or liquids and can, for example, be in the form of tablets, pills, dragees, gelatine capsules, solutions, syrups, suppositories, patches, inhalants, and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula I in compositions for oral administration is at least 0.5% by weight and can be up to 80% by weight with respect to the total weight of the composition.

For the preferred oral compositions, the daily dosage is in the range 0.01 to 1000 milligrams (mg) of compounds of formula I. In compositions for parenteral administration, the quantity of compound of formula I present is at least 0.5% by weight and can be up to 33% by weight with respect to the total weight of the composition. For the preferred parenteral compositions, the dosage unit is in the range 0.01 mg to 1000 mg of compounds of formula I.

The daily dose can fall within a wide range of dosage units of compound of formula I is generally in the range 0.01 to 1000 mg. However, it should be understood that the specific doses could be adapted to particular cases depending on the individual requirements, at the physician's discretion.

The compounds of the invention may be co-administered with another therapeutic agent most likely from a different therapeutic area.

Co-administration in this context means the dosing either of components, which are formulated together as a single dosage form; or the administration of separately formulated agents at substantially the same time, or sequential dosing of a compound of the invention followed by a therapeutic agent of a different therapeutic area.

In this context suitable examples of therapeutic agents may include, but are not limited to, histamine H₁ antagonists such as cetirizine, histamine H₂ antagonists, histamine H₃ antagonists, leukotriene antagonists, PDE4 inhibitors such as 3-cyclo-propylmethoxy-4-difluoromethoxy-N-[3,5-di-chloropyrid-4-yl]-benzamide, muscarinic M3 antagonists, β₂ agonists, theophylline, sodium cromoglycate, anti-TNF antibodies such as certolizumab pegol or adalimumab, anti-IL6 antibodies, anti-IL17 antibodies, adhesion molecule inhibitors, inhibitors of cytokine synthesis such as P38 MAP kinase inhibitors and inhibitors of PI3 kinase, methotrexate.

The present invention concerns also processes for preparing the compounds of formula I.

The compounds of formula I according to the invention can be prepared analogously to conventional methods as understood by the person skilled in the art of synthetic organic chemistry.

The following processes description sets forth certain synthesis routes in an illustrative manner. Other alternative and/or analogous methods will be readily apparent to those skilled in this art.

Compounds of formula I may be prepared according to one of the following general methods.

In scheme 1, ketone (A) is condensed (step 1) with an alkyl chloroformate or an alkyl carbonate, for example dimethyl carbonate in the presence of a base such as sodium hydride (NaH) in a solvent such as tetrahydrofuran (THF) or 1-methyl-2-pyrrolidinone (NMP). The resulting β keto-ester (B) is treated (step 2) with an amidine or a guanidine salt, such as guanidine carbonate, under conventional or microwave heating in a solvent such as an alcohol, for example ethanol (EtOH), with or without an added base. The resulting intermediate (C) is then reacted (step 3) with a chlorinating agent, such as phosphoryl chloride (POCl₃), under conventional heating to give compound (D). Introduction of group A is effected by heating with AH with or without an added base, for example an organic base such as N,N-diisopropylethylamine (DIPEA) or triethylamine (Et₃N), in a solvent such as NMP or EtOH under conventional or microwave heating to provide compounds of formula I.

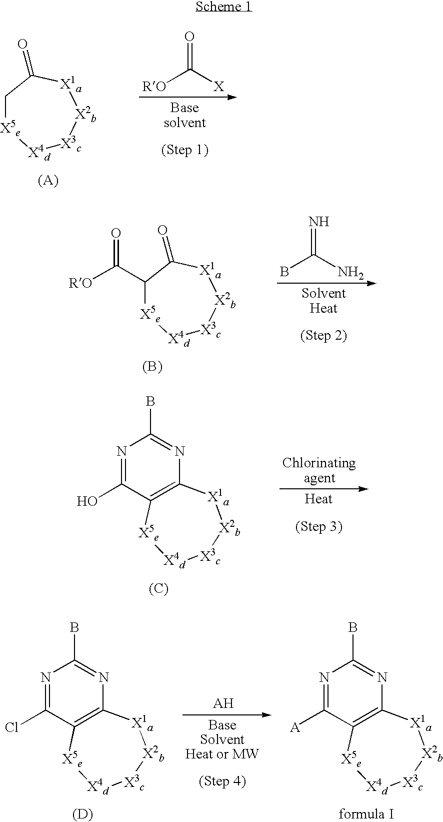

Alternatively a compound of formula (I) may be prepared from a dichloro derivative of formula (E):

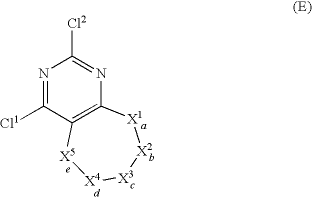

Initially introduction of the group A is effected by displacement of Cl¹ by heating with AH. Cl² may then be displaced by heating with B(H)LG, where LG is a leaving group such a p-methoxybenzyl, in the presence of a base, for example diisopropylethylamine in a solvent such as NMP. The group LG may be removed using methods known to those skilled in the art, such as heating in the presence of trifluoroacetic acid.

Scheme 2 describes the preparation of β substituted Ketone (A′). An α, β unsaturated ketone can be reacted with alkenyl or aryl or heteroaryl boronic acids (Rb(OH)₂) to lead to the formation of a ketone bearing alkenyl, aryl or heteroaryl substituents at the β position. For the preparation of ketones bearing alkyl or cycloalkyl substituents at the β position the reaction can take place in the presence of an organocopper complex (R₂CuLi).

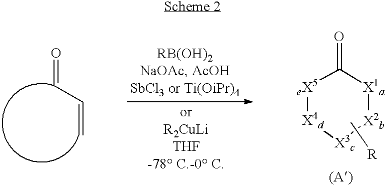

The present invention also relates to synthetic intermediates geometrical isomers, enantiomers, diastereoisomers, pharmaceutically acceptable salts and all possible mixtures thereof.

Specific synthetic intermediates are selected from the group consisting of:
tert-butyl [1-(2-amino-6,7-dihydro-5H-cyclopenta[d]pyrimidin-4-yl)pyrrolidin-3-yl]carbamate;
tert-butyl [1-(2-amino-6,7,8,9-tetrahydro-5H-cyclohepta[d]pyrimidin-4-yl)pyrrolidin-3-yl]carbamate;
tert-butyl [1-(2-amino-6-phenyl-5,6,7,8-tetrahydroquinazolin-4-yl)pyrrolidin-3-yl]carbamate;
methyl 2-oxo-4-phenylcyclohexanecarboxylate;
2-amino-7-phenyl-5,6,7,8-tetrahydroquinazolin-4-ol;
4-chloro-7-phenyl-5,6,7,8-tetrahydroquinazolin-2-amine;
tert-butyl [1-(2-amino-7-phenyl-5,6,7,8-tetrahydroquinazolin-4-yl)azetidin-3-yl]carbamate;
tert-butyl [1-(2-amino-7-phenyl-5,6,7,8-tetrahydroquinazolin-4-yl)pyrrolidin-3-yl]carbamate;
tert-butyl (4aR*,7aR*)-6-(2-amino-7-phenyl-5,6,7,8-tetrahydroquinazolin-4-yl)octahydro-1H-pyrrolo[3,4-b]pyridine-1-carboxylate;
tert-butyl 4-(2-amino-7-phenyl-5,6,7,8-tetrahydroquinazolin-4-yl)-2-methylpiperazine-1-carboxylate;
4-(1-benzyl-1,7-diazaspiro[4.4]non-7-yl)-7-phenyl-5,6,7,8-tetrahydroquinazolin-2-amine;
methyl 4-(3-chlorophenyl)-2-oxocyclohexanecarboxylate;
2-amino-7-(3-chlorophenyl)-5,6,7,8-tetrahydroquinazolin-4-ol;
tert-butyl 4-[2-amino-7-(3-chlorophenyl)-5,6,7,8-tetrahydroquinazolin-4-yl]-1,4-diazepane-1-carboxylate;
tert-butyl (4aR*,7aR*)-6-[2-amino-7-(3-chlorophenyl)-5,6,7,8-tetrahydroquinazolin-4-yl]octahydro-1H-pyrrolo[3,4-b]pyridine-1-carboxylate;
2-amino-7,7-dimethyl-5,6,7,8-tetrahydroquinazolin-4-ol;
4-chloro-7,7-dimethyl-5,6,7,8-tetrahydroquinazolin-2-amine;
tert-butyl 4-(2-amino-7,7-dimethyl-5,6,7,8-tetrahydroquinazolin-4-yl)-1,4-diazepane-1-carboxylate;
tert-butyl [1-(2-amino-7,7-dimethyl-5,6,7,8-tetrahydroquinazolin-4-yl)pyrrolidin-3-yl]carbamate;
tert-butyl (4aR*,7aR*)-6-(2-amino-7,7-dimethyl-5,6,7,8-tetrahydroquinazolin-4-yl)octahydro-1H-pyrrolo[3,4-b]pyridine-1-carboxylate;
tert-butyl {1-[2-amino-7-(3-chlorophenyl)-5,6,7,8-tetrahydroquinazolin-4-yl]pyrrolidin-3-yl}carbamate;
tert-butyl 4-(2-amino-7,7-dimethyl-5,6,7,8-tetrahydroquinazolin-4-yl)-2-methylpiperazine-1-carboxylate;
methyl 4-(2-chlorophenyl)-2-oxocyclohexanecarboxylate;
2-amino-7-(2-chlorophenyl)-5,6,7,8-tetrahydroquinazolin-4-ol;
4-chloro-7-(2-chlorophenyl)-5,6,7,8-tetrahydroquinazolin-2-amine;
tert-butyl 4-[2-amino-7-(2-chlorophenyl)-5,6,7,8-tetrahydroquinazolin-4-yl]-1,4-diazepane-1-carboxylate;
tert-butyl (4aR*,7aR*)-6-[2-amino-7-(2-chlorophenyl)-5,6,7,8-tetrahydroquinazolin-4-yl]octahydro-1H-pyrrolo[3,4-b]pyridine-1-carboxylate;
tert-butyl {1-[2-amino-7-(2-chlorophenyl)-5,6,7,8-tetrahydroquinazolin-4-yl]pyrrolidin-3-yl}carbamate;
2-amino-7-(4-fluorophenyl)-5,6,7,8-tetrahydroquinazolin-4-ol;
4-chloro-7-(4-fluorophenyl)-5,6,7,8-tetrahydroquinazolin-2-amine;
tert-butyl (4aR*,7aR*)-6-(2-amino-5,6,7,8-tetrahydroquinazolin-4-yl)octahydro-1H-pyrrolo[3,4-b]pyridine-1-carboxylate;
methyl 2-oxo-4-pyridin-2-ylcyclohexanecarboxylate;
2-amino-7-pyridin-2-yl-5,6,7,8-tetrahydroquinazolin-4-ol;
4-chloro-7-pyridin-2-yl-5,6,7,8-tetrahydroquinazolin-2-amine;
methyl 4-(5-chloro-2-thienyl)-2-oxocyclohexanecarboxylate;
methyl 2-(5-chloro-2-thienyl)-6-oxocyclohexanecarboxylate;
2-amino-7-(5-chloro-2-thienyl)-5,6,7,8-tetrahydroquinazolin-4-ol;
4-chloro-7-(5-chloro-2-thienyl)-5,6,7,8-tetrahydroquinazolin-2-amine;
4-chloro-5-(5-chloro-2-thienyl)-5,6,7,8-tetrahydroquinazolin-2-amine.

Further specific synthetic intermediates are selected from the group consisting of:
tert-butyl (4aR,7aR)-6-[2-amino-7-(4-fluorophenyl)-5,6,7,8-tetrahydroquinazolin-4-yl]octahydro-1H-pyrrolo[3,4-b]pyridine-1-carboxylate;
tert-butyl (2-{[2-amino-7-(4-fluorophenyl)-5,6,7,8-tetrahydroquinazolin-4-yl]amino}ethyl)methylcarbamate;
tert-butyl [1-(2-amino-7,7-dimethyl-5,6,7,8-tetrahydroquinazolin-4-yl)azetidin-3-yl]carbamate;
4-chloro-7-isobutyl-5,6,7,8-tetrahydroquinazolin-2-amine;
2-amino-6,6-dimethyl-5,6,7,8-tetrahydroquinazolin-4-ol;
4-chloro-6,6-dimethyl-5,6,7,8-tetrahydroquinazolin-2-amine;
tert-butyl {2-[(2-amino-7,7-dimethyl-5,6,7,8-tetrahydroquinazolin-4-yl)amino]ethyl}methylcarbamate;
tert-butyl 5-(2-amino-7,7-dimethyl-5,6,7,8-tetrahydroquinazolin-4-yl)hexahydropyrrolo[3,4-c]pyrrole-2(1H)-carboxylate;
tert-butyl 4-(2-amino-6,6-dimethyl-5,6,7,8-tetrahydroquinazolin-4-yl)-2-methylpiperazine-1-carboxylate;
2-amino-8,8-dimethyl-5,6,7,8-tetrahydroquinazolin-4-ol;
4-chloro-8,8-dimethyl-5,6,7,8-tetrahydroquinazolin-2-amine;
tert-butyl 4-(2-amino-8,8-dimethyl-5,6,7,8-tetrahydroquinazolin-4-yl)-2-methylpiperazine-1-carboxylate;
tert-butyl [1-(2-amino-8,8-dimethyl-5,6,7,8-tetrahydroquinazolin-4-yl)azetidin-3-yl]carbamate;
tert-butyl (4aR,7aR)-6-(2-amino-8,8-dimethyl-5,6,7,8-tetrahydroquinazolin-4-yl)octahydro-1H-pyrrolo[3,4-b]pyridine-1-carboxylate;
tert-butyl 4-[(2-amino-8,8-dimethyl-5,6,7,8-tetrahydroquinazolin-4-yl)amino]piperidine-1-carboxylate;
tert-butyl 3-[(2-amino-8,8-dimethyl-5,6,7,8-tetrahydroquinazolin-4-yl)amino]pyrrolidine-1-carboxylate;
methyl 2-oxospiro[5.5]undecane-3-carboxylate;
2′-amino-5′,8′-dihydro-6′H-spiro[cyclohexane-1,7′-quinazolin]-4′-ol;
4′-chloro-5′,8′-dihydro-6′H-spiro[cyclohexane-1,7′-quinazolin]-2′-amine;
tert-butyl 2-amino-4-hydroxy-5,8-dihydropyrido[3,4-d]pyrimidine-7(6H)-carboxylate;
tert-butyl 2-[(2,2-dimethylpropanoyl)amino]-4-hydroxy-5,8-dihydropyrido[3,4-d]pyrimidine-7(6H)-carboxylate;
N-(4-hydroxy-5,6,7,8-tetrahydropyrido[3,4-d]pyrimidin-2-yl)-2,2-dimethylpropanamide;
N-[7-(5-cyanopyridin-2-yl)-4-hydroxy-5,6,7,8-tetrahydropyrido[3,4-d]pyrimidin-2-yl]-2,2-dimethylpropanamide;
N-[4-chloro-7-(5-cyanopyridin-2-yl)-5,6,7,8-tetrahydropyrido[3,4-d]pyrimidin-2-yl]-2,2-dimethylpropanamide;
N-[7-(5-cyanopyridin-2-yl)-4-(4-methylpiperazin-1-yl)-5,6,7,8-tetrahydropyrido[3,4-d]pyrimidin-2-yl]-2,2-dimethylpropanamide;
N-{4-hydroxy-7-[4-(trifluoromethyl)pyrimidin-2-yl]-5,6,7,8-tetrahydropyrido[3,4-d]pyrimidin-2-yl}-2,2-dimethylpropanamide;
N-{4-chloro-7-[4-(trifluoromethyl)pyrimidin-2-yl]-5,6,7,8-tetrahydropyrido[3,4-d]pyrimidin-2-yl}-2,2-dimethylpropanamide;
2,2-dimethyl-N-{4-(4-methylpiperazin-1-yl)-7-[4-(trifluoromethyl)pyrimidin-2-yl]-5,6,7,8-tetrahydropyrido[3,4-d]pyrimidin-2-yl}propanamide;
tert-butyl (4aR,7aR)-6-(2′-amino-5′,8′-dihydro-6′H-spiro[cyclohexane-1,7′-quinazolin]-4′-yl)octahydro-1H-pyrrolo[3,4-b]pyridine-1-carboxylate;
tert-butyl [1-(2′-amino-5′,8′-dihydro-6′H-spiro[cyclohexane-1,7′-quinazolin]-4′-yl)azetidin-3-yl]methylcarbamate;
tert-butyl [1-(2-amino-7-isobutyl-5,6,7,8-tetrahydroquinazolin-4-yl)pyrrolidin-3-yl]carbamate;
methyl 4-isopropyl-2-oxocyclohexanecarboxylate;
2-amino-7-isopropyl-5,6,7,8-tetrahydroquinazolin-4-ol;
4-chloro-7-isopropyl-5,6,7,8-tetrahydroquinazolin-2-amine;
tert-butyl (4aR,7aR)-6-(2-amino-7-isopropyl-5,6,7,8-tetrahydroquinazolin-4-yl)octahydro-1H-pyrrolo[3,4-b]pyridine-1-carboxylate;
tert-butyl [1-(2-amino-7-isopropyl-5,6,7,8-tetrahydroquinazolin-4-yl)azetidin-3-yl]methylcarbamate.

The following examples are provided for illustrative purposes only and are not intended, nor should they be construed, as limiting the invention in any manner. Those skilled in the art will appreciate that routine variations and modifications of the following examples can be made without exceeding the spirit or scope of the invention.

Unless specified otherwise in the examples, characterization of the compounds is performed according to (LCMS) liquid chromatography mass spectra, preparative liquid chromatography LC, NMR, and silica gel chromatography methods.

NMR spectra are recorded on Bruker AV300 and DRX 400 spectrometers at 300 and 400 MHz respectively.

Chromatographic separations are performed on Davisil 5 μM silica gel.

The Waters mass spectrometers used are of model ZMD or ZQ both Waters.

Various reactions were performed in an Emrys Optimiser microwave reactor.

The following abbreviations are used in the examples:

### DCM—Dichloromethane

### DIPEA—N,N-Diisopropylethylamine

### DMAP—4-Dimethylaminopyridine

DMSO—Dimethyl sulphoxide

### DMF—N,N-Dimethylformamide

d₆-DMSO—d₆-Dimethyl sulphoxide
EtOAc—Ethyl acetate

### EtOH—Ethanol

ESI—Electrospray ionization
K₂CO₃—Potassium carbonate

### MeOH—Methanol

d₄-MeOH—d₄-Methanol
MTBE—Methyl tert-butyl ether
NMP—1-Methyl-2-pyrrolidinone
NMR—Nuclear magnetic resonance
MgSO₄—Magnesium sulfate
NaHCO₃—Sodium bicarbonate
NaH—Sodium hydride
NaCl—Sodium chloride
NaOH—Sodium hydroxide
RT—Retention time

### TEA—Triethylamine

### THF—Tetrahydrofuran

TLC—Thin layer chromatography
TFA—Trifluoroacetic acid
ESI—Electrospray ionization

### Pos—Positive

### Neg—Negative

The IUPAC names of compounds are generated using ACD (Labs Release: 9.00, product version: 9.04).

All the reagents, solvents, catalysts for which the synthesis is not described are purchased from chemical vendors such Sigma-Aldrich, Fluke, Lancaster, however some known reaction intermediates, for which the registry numbers (RN) are mentioned, are prepared in-house following known procedures.

The LCMS conditions used to obtain the retention times (RT) are described herein:

### 1. LCMS Conditions (pH 2)

HP1100 (Diode Array) linked to a Finnigan LC-Q Mass Spectrometer, ESI mode with Pos/Neg ionisation or Waters 2695 linked to a Waters ZMD Mass Spectrometer, ESI mode with Pos/Neg ionisation.

| Column: | Phenomenex Luna C18(2) 100 × 4.6 mm, 5 μm | |
|---|---|---|
| | particle size Analytical column | |
| Column temp: | 35° C. | |
| Mobile phase: | A: Water + 0.08% formic acid | |
| | B: Acetonitrile + 0.08% formic acid | |
| Flow rate: | 3 ml/min | |
| | Time (min) | % Composition B |
| Gradient: | 0 | 5 |
| | 4.40 | 95 |
| | 5.30 | 95 |
| | 5.32 | 5 |
| | 6.50 | 5 |

| | |
|---|---|
| Run time: | 6.50 min |
| Typical injection volume: | 10 μl |
| Detector wavelength: | DAD 200-400 nm |

### 2. LCMS Conditions (pH 5.8)

HP1100 (Diode Array) linked to a Finnigan LC-Q Mass Spectrometer, ESI mode with Pos/Neg ionization or Waters 2695 linked to a Waters ZMD Mass Spectrometer, ESI mode with Pos/Neg ionization.

| Column: | Phenomenex Luna C18(2) 100 × 4.6 mm, 5 μm | |
|---|---|---|
| | particle size Analytical column | |
| Column temp: | 35° C. | |
| Mobile phase: | A: 5 mM NH₄OAc pH 5.8 | |
| | B: 95:5, MeCN:100 mM NH₄OAc pH 5.8 | |
| Flow rate: | 3 ml/min | |
| | Time (min) | % Composition B |
| Gradient: | 0 | 5 |
| | 4.40 | 95 |
| | 5.30 | 95 |
| | 5.32 | 5 |
| | 6.50 | 5 |

| | |
|---|---|
| Run time: | 6.50 min |
| Typical injection volume: | 10 μl |
| Detector wavelength: | DAD 200-400 nm |

The following preparative LC conditions are used to purify compounds as described herein:

### Preparative LC Conditions (pH 2.5)

Waters autopreparative mass and UV directed: ZQ mass spectrometer, 996 PDA, 2525 pump and 2767 autosampler/fraction collector and 2757 fraction collector.

| | |
|---|---|
| Column: | Phenomenex Luna C18(2) 250 × 21.2 mm, 5 μm |
| | particle size prep column |
| Column temp: | Ambient |
| Mobile phase: | A: Water + 0.08% formic acid |
| | B: Acetonitrile + 0.08% formic acid |
| Flow rate: | 25 ml/min |
| Gradient: | Variable - depends on retention time |
| | of sample in LC-MS analysis |
| Run time: | 20 min |
| Injection volume: | 1 ml at 50 mg/ml (typically) |
| Detector wavelength: | 200 to 400 nm |

### Preparative LC Conditions (pH 5.8)

Waters autopreparative mass and UV directed: ZQ mass spectrometer, 996 PDA, 2525 pump and 2767 autosampler/fraction collector and 2757 fraction collector.

| | |
|---|---|
| Column: | Phenomenex Luna C18(2) 250 × 21.2 mm, 5 μm |
| | particle size prep column |
| Column temp: | Ambient |
| Mobile phase: | A: 10 mM ammonium acetate pH 5.8 |
| | B: 5:95, 200 mM ammonium acetate |
| | pH 5.8:Acetonitrile |
| Flow rate: | 25 ml/min |
| Gradient: | Variable - depends on retention time |
| | of sample in LC-MS analysis |
| Run time: | 20 min |
| Injection volume: | 1 ml at 50 mg/ml (typically) |
| Detector wavelength: | 200 to 400 nm |

### EXAMPLE 1

### Synthesis of tert-butyl [1-(2-amino-6,7-dihydro-5H-cyclopenta[d]pyrimidin-4-yl)pyrrolidin-3-yl]carbamate (Intermediate 1)

4-Chloro-6,7-dihydro-5H-cyclopenta[d]pyrimidin-2-amine (CAS No 5461-89-2) (54 mg), tert-butyl pyrrolidin-3-ylcarbamate (63 mg) and DIPEA (0.059 ml) are treated with absolute EtOH (2 ml) and heated under microwave irradiation at 150° C. for 30 mins. The solution is concentrated in vacuo and the residue purified by flash chromatography, eluting with DCM-MeOH 95:5, then 94:6 to afford the title compound as a colourless crystalline solid (93 mg, 90%). R_{f} (DCM-MeOH 94:6) 0.29. LCMS 320 [M+H]⁺, RT 1.98 mins (pH 2.5). ¹H NMR 300 MHz (CDCl₃) (δ ppm): 6.07 (2H, bm), 4.65 (1H, m), 4.28 (1H, m), 3.95 (1H, dd), 3.80 (2H, m), 3.62 (1H, m), 3.02 (2H, t), 2.88 (2H, t), 1.87-2.30 (4H, m), 1.45 (9H, s).

Intermediates 2 and 3 are prepared in a similar manner to the method described for Intermediate 1 in Example 1. The reagents used and the results obtained are tabulated below (Table 1). The free base of the compounds is obtained unless otherwise stated.

TABLE 1

| | | | | ¹H NMR |
|---|---|---|---|---|
| | | Starting | | (Solvent, |
| Int. No | IUPAC Name | Materials | LCMS | δ ppm) |
| 2 | tert-butyl [1-(2- | 4-chloro-6,7,8,9- | 348 [M + H]⁺, | CDCl₃ 6.00 (2H, bm), 5.10 (1H, m), |
| | amino-6,7,8,9- | tetrahydro-5H- | RT 2.16 mins | 4.22 (1H, bm), 3.70-3.90 (2H, m), |
| | tetrahydro-5H- | cyclohepta[d]pyrimidin- | (pH | 3.65 (1H, m), 3.51 (1H, dd), |
| | cyclohepta[d]pyrimidin- | 2-amine | 2.5) | 2.81 (2H, m), 2.65 (2H, m), 2.16 (1H, |
| | 4- | (CAS RN | | m), 1.55-2.02 (7H, m), 1.45 (9H, |
| | yl)pyrrolidin-3- | 569657-96-1), | | s) |
| | yl]carbamate | tert-butyl | | |
| | | pyrrolidin-3- | | |
| | | ylcarbamate | | |
| 3 | tert-butyl [1-(2- | 4-chloro-6- | 410 [M + H]⁺, | CDCl₃+ d₄-MeOH 7.20-7.40 (5H, |
| | amino-6-phenyl- | phenyl-5,6,7,8- | RT 2.52 mins | m), 4.19 (1H, bm), 3.40-3.92 (5H, |
| | 5,6,7,8- | tetrahydroquinazolin- | (pH | m), 2.60-2.97 (4H, m), |
| | tetrahydroquinazolin- | 2-amine | 2.5) | 1.70-2.28 (5H, m), 1.43 (9H, s) |
| | 4- | (CAS RN 13658- | | |
| | yl)pyrrolidin-3- | 23-6), tert-butyl | | |
| | yl]carbamate | pyrrolidin-3- | | |
| | | ylcarbamate | | |

* Int. No means Intermediate Number

### EXAMPLE 2

### Synthesis of methyl 2-oxo-4-phenylcyclohexanecarboxylate (Intermediate 4)

3-Phenylcyclohexanone (1.193 g) is dissolved in dry THF under N₂ and NaH 60% dispersion in mineral oil (329 mg) added. After stirring at room temperature for 30 mins, dimethyl carbonate (0.693 ml) is added, and the mixture heated at 75° C. for 17 hrs. The solvent is removed in vacuo, the residual oil partitioned between DCM (50 ml) and saturated brine (25 ml), and the 2-phase mixture filtered through Celite. The organic phase is separated, dried (MgSO₄) and concentrated in vacuo. Purification of the residual oil by flash chromatography, eluting with DCM-Heptane 1:1, then 5:2 affords the title compound in a 3:1 ratio of regioisomers by NMR as a colourless oil (764 mg, 48%). R_{f} (DCM-Heptane 1:1) 0.17. LCMS 233 [M+H]⁺, RT 4.42 mins (pH 2.5). ¹H NMR 300 MHz (CDCl₃) (δ ppm): 12.18 (1H, s), 7.18-7.38 (5H, m), 3.79 (3H, s), 2.90 (1H, m), 2.25-2.60 (4H, m), 2.00 (1H, m), 1.70 (1H, m).

### EXAMPLE 3

### Synthesis of 2-amino-7-phenyl-5,6,7,8-tetrahydroquinazolin-4-ol (Intermediate 5)

Intermediate 4 (764 mg) is dissolved in abs. EtOH (15 ml), guanidine carbonate (593 mg) is added, and the mixture heated at 78° C. for 18 hrs. The EtOH is removed in vacuo, and the residual solid suspended in DCM (10 ml) and water (10 ml). The undissolved solids are collected by filtration, washed with water (5 ml) and dried in a vacuum oven at 40° C. to afford the title compound in a 3:1 ratio of regioisomers by NMR as a brown solid (734 mg, 93%). LCMS 242 [M+H]⁺, RT 1.79 mins (pH 2.5). ¹H NMR 300 MHz (d₆-DMSO) (δ ppm): 7.15-7.35 (5H, m), 6.35 (2H, bm), 3.30 (1H, bm+H₂0), 2.87 (1H, m), 2.35-2.60 (3H, m), 2.22 (1H, m), 1.90 (1H, m), 1.70 (1H, m).

### EXAMPLE 4

### Synthesis of 4-chloro-7-phenyl-5,6,7,8-tetrahydroquinazolin-2-amine (Intermediate 6)

Intermediate 5 (732 mg) is partially dissolved in DCM-MeOH (approx. 5:1, 15 ml) and HCl in dioxane 4.0M (0.76 ml) is added, and the mixture sonicated with warming to achieve solution. The solvents are removed in vacuo and the residue is dried. Anhydrous dioxane (10 ml) is then added, followed by POCl₃ (11 ml) and the mixture is heated under N₂ at 110° C. for 75 mins. The volatiles are then removed in vacuo, the residue is quenched with ice/water and neutralised to approx. pH 14 with 48% NaOH solution. Then EtOAc (30 ml) is added, and the undissolved product collected by filtering off through a porosity 4 sinter. The EtOAc phase is separated, dried (MgSO₄) and concentrated in vacuo. These two solid samples are combined and the desired regioisomer separated by flash chromatography, eluting with EtOAc-Heptane 1:3, then 1:2 affording the title compound as a colourless solid (396 mg, 50%). R_{f} (EtOAc-Heptane 1:3) 0.24. LCMS 260 [M+H]⁺, RT 3.57 mins (pH 2.5). ¹H NMR 300 MHz (CDCl₃) (δ ppm): 7.20-7.40 (5H, m), 4.92 (2H, bs), 2.78-3.10 (4H, m), 2.65 (1H, m), 2.20 (1H, m), 1.90 (1H, m).

Intermediates 7 to 11 are prepared in a similar manner to the method described for Intermediate 1 in Example 1. The reagents used and the results obtained are tabulated below (Table 2). The free base of the compounds is obtained unless otherwise stated.

TABLE 2

| | | | | ¹H NMR |
|---|---|---|---|---|
| Int. | | Starting | | (Solvent, |
| No | IUPAC Name | Materials | LCMS | δ ppm) |
| 7 | tert-butyl [1-(2- | Intermediate | 396 [M + H]⁺, | d₄-MeOH |
| | amino-7-phenyl- | 6, tert-butyl | RT 2.48 mins | 7.18-7.45 (5H, m), 5.36 (3H, s), |
| | 5,6,7,8- | azetidin-3- | (pH 2.5) | 4.50-4.70 (3H, m), |
| | tetrahydroquinazolin- | ylcarbamate | | 4.09-4.30 (2H, m), |
| | 4-yl)azetidin-3- | | | 2.54-3.05 (5H, m), |
| | yl]carbamate | | | 2.15 (1H, m), 1.85 (1H, |
| | | | | m), 1.45 (9H, s). |
| 8 | tert-butyl [1-(2- | Intermediate | 410 [M + H]⁺, | CDCl₃ 7.15-7.39 (5H, |
| | amino-7-phenyl- | 6, tert-butyl | RT 2.46 mins | m), 6.13 (2H, bs), |
| | 5,6,7,8- | pyrrolidin-3- | (pH | 4.83 (1H, dd), 4.22 (1H, m), |
| | tetrahydroquinazolin- | ylcarbamate | 2.5) | 3.55-4.08 (4H, m), |
| | 4-yl)pyrrolidin-3- | | | 2.73-3.12 (5H, m), |
| | yl]carbamate | | | 1.65-2.32 (4H, m), |
| | | | | 1.45 (9H, s) |
| 9 | tert-butyl | Intermediate | 450 [M + H]⁺, | CDCl₃ 7.12-7.40 (5H, |
| | (4aR*,7aR*)-6-(2- | 6, tert-butyl | RT 2.73 mins | m), 6.00 (2H, bs), |
| | amino-7-phenyl- | (4aR*,7aR*)- | (pH | 4.72 (1H, m), 4.02 (1H, m), |
| | 5,6,7,8- | octahydro- | 2.5) | 3.85 (1H, m), 3.70 (2H, |
| | tetrahydroquinazolin- | 1H- | | m), 3.54 (1H, dt), |
| | 4-yl)octahydro-1H- | pyrrolo[3,4- | | 2.62-3.15 (6H, m), |
| | pyrrolo[3,4- | b]pyridine-1- | | 1.65-2.30 (5H, m), 1.47 (9H, |
| | b]pyridine-1- | carboxylate | | s), 1.21-1.55 (2H, m) |
| | carboxylate | | | |
| 10 | tert-butyl 4-(2-amino- | Intermediate | 424 [M + H]⁺, | CDCl₃ 7.18-7.38 (5H, |
| | 7-phenyl-5,6,7,8- | 6, tert-butyl | RT 2.68 mins | m), 5.52 (2H, bd), |
| | tetrahydroquinazolin- | 2- | (pH | 4.31 (1H, m), |
| | 4-yl)-2- | methylpiperazine- | 2.5) | 3.71-4.07 (3H, m), 3.32 (1H, m), |
| | methylpiperazine-1- | 1- | | 2.51-3.20 (7H, m), |
| | carboxylate | carboxylate | | 2.13 (1H, m), 1.78 (1H, |
| | | | | m), 1.48 (9H, s), |
| | | | | 1.27 (1.5H, d), 1.15 (1.5H, |
| | | | | d) |
| 11 | 4-(1-benzyl-1,7- | Intermediate | 440 [M + H]⁺, | CDCl₃ |
| | diazaspiro[4.4]non- | 6, 1-benzyl- | RT 1.75 mins | 7.17-7.38 (10H, m), 5.54 (2H, |
| | 7-yl)-7-phenyl- | 1,7- | (pH | bs), 3.35-4.02 (7H, |
| | 5,6,7,8- | diazaspiro[4.4]nonane | 2.5) | m), 2.59-3.10 (8H, |
| | tetrahydroquinazolin- | | | m), 1.63-2.24 (6H, m) |
| | 2-amine | | | |

Intermediate 12 is prepared in a similar manner to the method described for Intermediate 4 in Example 2.

### Synthesis of methyl 4-(3-chlorophenyl)-2-oxocyclohexanecarboxylate (Intermediate 12)

Starting from 3-(3-chlorophenyl)cyclohexanone (CAS RN 335259-42-2). R_{f} (DCM-Heptane 2:3) 0.51. LCMS 251 [M-Me], RT 3.79 mins (pH 2.5). ¹H NMR 300 MHz (CDCl₃) (δ ppm): 12.55 (1H, s), 7.00-7.28 (4H, m), 3.90 (1H, m), 3.55 (3H, s), 2.39 (2H, m), 1.45-2.00 (4H, m).

Intermediate 13 is prepared in a similar manner to the method described for Intermediate 5 in Example 3.

### Synthesis of 2-amino-7-(3-chlorophenyl)-5,6,7,8-tetrahydroquinazolin-4-ol (Intermediate 13)

Starting from Intermediate 12. LCMS 276 [M+H]⁺, RT 2.01 mins (pH 2.5). ¹H NMR 300 MHz (d₆-DMSO) (δ ppm): 6.98-7.30 (4H, m), 6.38 (2H, bm), 3.88 (1H, m), 3.42 (1H, m), 2.38 (2H, m), 1.85 (1H, m), 1.68 (1H, m), 1.50 (1H, m), 1.40 (1H, m).

### EXAMPLE 5

### Synthesis of tert-butyl 4-[2-amino-7-(3-chlorophenyl)-5,6,7,8-tetrahydroquinazolin-4-yl]-1,4-diazepane-1-carboxylate (Intermediate 14)

Intermediate 13 (160 mg) is suspended in POCl₃ (2 ml) and heated under N₂ at 105° C. for 35 mins. The excess POCl₃ is removed in vacuo, the residue quenched with ice and neutralised to pH 14 with 48% NaOH solution. The aqueous phase is extracted with EtOAc (2×20 ml), the organic phases dried (MgSO₄) and concentrated in vacuo to afford the crude chloro pyrimidine as a buff solid (109 mg, 64%). The chloro pyrimidine (37.1 mg) is suspended in abs. EtOH (2 ml), DIPEA (0.035 ml) and tert-butyl 1,4-diazepane-1-carboxylate (0.039 ml) added and the reaction heated under microwave irradiation at 170° C. for 60 mins. The resulting solution is concentrated in vacuo, and the residue purified by flash chromatography, eluting with DCM-MeOH 95:5 to afford the title compound as a colourless glass (21 mg, 36%). LCMS 458 [M+H]⁺, RT 2.53 mins (pH 2.5). ¹H NMR 300 MHz (CDCl₃) (δ ppm): 7.10-7.23 (2H, m), 7.00 (1H, s), 6.90 (1H, m), 4.70 (2H, bm), 4.00 (1H, m), 2.91-3.67 (8H, m), 2.72 (2H, m), 1.51-2.15 (6H, m), 1.42 (9H, s).

Intermediate 15 is prepared in a similar manner to the method described for Intermediate 14 in Example 5.

### Synthesis of tert-butyl (4aR*,7aR*)-6-[2-amino-7-(3-chlorophenyl)-5,6,7,8-tetrahydroquinazolin-4-yl]octahydro-1H-pyrrolo[3,4-b]pyridine-1-carboxylate (Intermediate 15)

Starting from Intermediate 13 and using tert-butyl (4aR*,7aR*)-octahydro-1H-pyrrolo[3,4-b]pyridine-1-carboxylate. LCMS 484 [M+H]⁺, RT 2.81 mins (pH 2.5). ¹H NMR 300 MHz (CDCl₃) (δ ppm): 6.81-7.30 (4H, m), 5.28-5.73 (2H, bm), 3.80-4.82 (5H, bm), 3.12-3.69 (4H, m), 2.55-2.85 (3H, m), 2.13-2.38 (1H, m), 1.85-2.09 (2H, m), 1.20-1.75 (13H, m).

Intermediate 16 is prepared in a similar manner to the method described for Intermediate 5 in Example 3.

### Synthesis of 2-amino-7,7-dimethyl-5,6,7,8-tetrahydroquinazolin-4-ol (Intermediate 16)

Starting from methyl 4,4-dimethyl-2-oxocyclohexanecarboxylate (CAS RN 32767-46-7). LCMS 194 [M+H]⁺, RT 1.54 mins (pH 2.5). ¹H NMR 300 MHz (d₆-DMSO) (δ ppm): 10.82 (1H, bs), 6.23 (2H, bs), 2.20 (2H, t), 2.10 (2H, s), 1.38 (2H, t), 0.90 (6H, s).

Intermediate 17 is prepared in a similar manner to the method described for Intermediate 6 in Example 4.

### Synthesis of 4-chloro-7,7-dimethyl-5,6,7,8-tetrahydroquinazolin-2-amine (Intermediate 17)

Starting from Intermediate 16. LCMS 212 [M+H]⁺, RT 3.05 mins (pH 2.5). ¹H NMR 300 MHz (CDCl₃) (δ ppm): 4.92 (2H, bs), 2.62 (2H, t), 2.44 (2H, s), 1.60 (2H, t), 1.00 (6H, s).

Intermediates 18-20 are prepared in a similar manner to the method described for Intermediate 1 in Example 1. The reagents used and the results obtained are tabulated below (Table 3). The free base of the compounds is obtained unless otherwise stated.

TABLE 3

| Int. | | | | ¹H NMR |
|---|---|---|---|---|
| No | IUPAC Name | Starting Materials | LCMS | (Solvent, δ ppm) |
| 18 | tert-butyl 4-(2- | Intermediate 17, | 376 [M + H]⁺, | CDCl₃ 10.05 (2H, bm), |
| | amino-7,7- | tert-butyl 1,4- | RT | 8.40 (2H, HCOOH), |
| | dimethyl- | diazepane-1- | 2.37 mins | 3.72-3.95 (4H, m), 3.62 (2H, m), |
| | 5,6,7,8- | carboxylate | (pH | 3.28-3.45 (2H, m), 2.45-2.60 (4H, |
| | tetrahydroquinazolin- | | 2.5) | m), 1.90 (2H, m), 1.51 (2H, t), |
| | 4-yl)- | | | 1.43 (9H, s), 1.03 (6H, s) |
| | 1,4- | | | |
| | diazepane-1- | | | |
| | carboxylate | | | |
| 19 | tert-butyl [1-(2- | Intermediate 17, | 362 [M + H]⁺, | CDCl₃ 5.56 (2H, bm), |
| | amino-7,7- | tert-butyl pyrrolidin- | RT | 4.80 (1H, m), 4.23 (1H, m), |
| | dimethyl- | 3-ylcarbamate | 2.33 mins | 3.94 (1H, dd), 3.70-3.89 (2H, m), |
| | 5,6,7,8- | | (pH | 3.59 (1H, dd), 2.67 (2H, t), |
| | tetrahydroquinazolin- | | 2.5) | 2.44 (2H, s), 2.15 (1H, m), |
| | 4- | | | 1.90 (1H, m), |
| | yl)pyrrolidin-3- | | | 1.40-1.56 (11H, m), 1.00 (6H, s). |
| | yl]carbamate | | | |
| 20 | tert-butyl | Intermediate 17, | 402 [M + H]⁺, | CDCl₃ 8.40 (2H, HCOOH), |
| | (4aR*,7aR*)-6- | tert-butyl | RT | 4.74 (1H, m), 3.53-4.10 (5H, |
| | (2-amino-7,7- | (4aR*,7aR*)- | 2.62 mins | m), 2.60-2.85 (3H, m), |
| | dimethyl- | octahydro-1H- | (pH | 2.47 (2H, dd), 2.21 (1H, m), |
| | 5,6,7,8- | pyrrolo[3,4- | 2.5) | 1.68-1.86 (2H, m), |
| | tetrahydroquinazolin- | b]pyridine-1- | | 1.22-1.63 (15H, m), 1.04 (3H, s), |
| | 4- | carboxylate | | 1.00 (3H, s) |
| | yl)octahydro- | | | |
| | 1H- | | | |
| | pyrrolo[3,4- | | | |
| | b]pyridine-1- | | | |
| | carboxylate | | | |

Intermediate 21 is prepared in a similar manner to the method described for Intermediate 14 in Example 5.

### Synthesis of tert-butyl {1-[2-amino-7-(3-chlorophenyl)-5,6,7,8-tetrahydroquinazolin-4-yl]pyrrolidin-3-yl}carbamate (Intermediate 21)

Starting from Intermediate 13 and tert-butyl pyrrolidin-3-ylcarbamate. R_{f} (DCM-MeOH 95:5) 0.26. LCMS 444 [M+H]⁺, RT 2.55 mins (pH 2.5). ¹H NMR 300 MHz (CDCl₃) (δ ppm): 7.07-7.25 (2H, m), 7.00 (1H, s), 6.88 (1H, m), 5.07 (2H, bd), 3.95-4.50 (3H, m), 3.15-3.75 (5H, m), 2.71 (2H, m), 1.50-2.20 (5H, m), 1.42 (9H, s).

Intermediate 22 is prepared in a similar manner to the method described for Intermediate 1 in Example 1.

### Synthesis of tert-butyl 4-(2-amino-7,7-dimethyl-5,6,7,8-tetrahydroquinazolin-4-yl)-2-methylpiperazine-1-carboxylate (Intermediate 22)

Starting from Intermediate 17 and tert-butyl 2-methylpiperazine-1-carboxylate. LCMS 376 [M+H]⁺, RT 2.46 mins (pH 2.5). ¹H NMR 300 MHz (CDCl₃) (δ ppm): 4.63 (2H, bs), 4.30 (1H, m), 3.90 (1H, m), 3.72 (1H, m), 3.60 (1H, m), 3.22 (3H, m), 3.00 (1H, dd), 2.81 (1H, dt), 2.50 (2H, m), 2.41 (2H, s), 1.48 (9H, s), 1.25 (3H, d), 1.02 (6H, s).

Intermediate 23 is prepared in a similar manner to the method described for Intermediate 4 in Example 2.

### Synthesis of methyl 4-(2-chlorophenyl)-2-oxocyclohexanecarboxylate (Intermediate 23)

Starting from 3-(2-chlorophenyl)cyclohexanone (CAS RN 141632-22-6). R_{f} (EtOAc-Heptane 1:9) 0.51. LCMS 267 [M+H]⁺, RT 4.61 mins (pH 2.5). ¹H NMR 300 MHz (CDCl₃) (δ ppm): 12.19 (1H, s), 7.37 (1H, d), 7.05-7.30 (3H, m), 3.79 (3H, s), 3.42 (1H, m), 2.65 (1H, dd), 1.68-2.43 (5H, m).

Intermediate 24 is prepared in a similar manner to the method described for Intermediate 5 in Example 3.

### Synthesis of 2-amino-7-(2-chlorophenyl)-5,6,7,8-tetrahydroquinazolin-4-ol carbonic acid salt (Intermediate 24)

Starting from Intermediate 23. LCMS 276 [M+H]⁺, RT 2.04 mins (pH 2.5). ¹H NMR 300 MHz (d₆-DMSO) (δ ppm): 7.90 (1H, bm+H₂CO₃), 7.10-7.48 (4H, m), 6.20 (2H, bs), 3.15-3.55 (3H, m), 2.15-2.45 (2H, m), 1.68-1.95 (2H, m).

Intermediate 25 is prepared in a similar manner to the method described for Intermediate 6 in Example 4.

### Synthesis of 4-chloro-7-(2-chlorophenyl)-5,6,7,8-tetrahydroquinazolin-2-amine (Intermediate 25)

Starting from Intermediate 24. LCMS 294 [M+H]⁺, RT 3.93 mins (pH 2.5). ¹H NMR 300 MHz (CDCl₃) (δ ppm): 7.40 (1H, d), 7.05-7.33 (3H, m), 5.00 (2H, bm), 3.50 (1H, m), 3.03 (1H, m), 2.62-2.93 (3H, m), 1.83-2.24 (2H, m).

Intermediates 26-28 are prepared in a similar manner to the method described for Intermediate 1 in Example 1. The reagents used and the results obtained are tabulated below (Table 4). The free base of the compounds is obtained unless otherwise stated.

TABLE 4

| | | | | ¹H NMR |
|---|---|---|---|---|
| Int. | | Starting | | (Solvent, |
| No | IUPAC Name | Materials | LCMS | δ ppm) |
| 26 | tert-butyl 4-[2- | Intermediate | 458 [M + H]⁺, | CDCl₃ 7.39 (1H, d), |
| | amino-7-(2- | 25, tert-butyl | RT | 7.12-7.30 (3H, m), 4.65 (2H, bs), |
| | chlorophenyl)- | 1,4- | 2.68 mins | 3.39-3.80 (8H, m), 3.23 (1H, m), |
| | 5,6,7,8- | diazepane-1- | (pH 2.5) | 3.05 (1H, dd), 2.47-2.77 (3H, m), |
| | tetrahydroquinazolin- | carboxylate | | 1.70-2.35 (4H, m), 1.45 (9H, s) |
| | 4-yl]- | | | |
| | 1,4- | | | |
| | diazepane-1- | | | |
| | carboxylate | | | |
| 27 | tert-butyl | Intermediate | 484 [M + H]⁺, RT | CDCl₃ 7.38 (1H, m), |
| | (4aR*,7aR*)- | 25, tert-butyl | 2.81 mins | 7.10-7.30 (3H, m), 5.28 (2H, bs), |
| | 6-[2-amino-7- | (4aR*,7aR*)- | (pH 2.5) | 3.40-4.85 (8H, m), 3.04 (1H, m), |
| | (2- | octahydro-1H- | | 2.57-2.93 (3H, m), 2.15 (1H, m), |
| | chlorophenyl)- | pyrrolo[3,4- | | 1.98 (1H, m), 1.60-1.81 (2H, m), |
| | 5,6,7,8- | b]pyridine-1- | | 1.20-1.53 (12H, m) |
| | tetrahydroquinazolin- | carboxylate | | |
| | 4- | | | |
| | yl]octahydro- | | | |
| | 1H- | | | |
| | pyrrolo[3,4- | | | |
| | b]pyridine-1- | | | |
| | carboxylate | | | |
| 28 | tert-butyl {1- | Intermediate | 444 [M + H]⁺, RT | CDCl₃ 7.39 (1H, d), |
| | [2-amino-7-(2- | 25, tert-butyl | 2.67 mins | 7.12-7.29 (3H, m), 5.50 (2H, bm), 4.75 (1H, |
| | chlorophenyl)- | pyrrolidin-3- | (pH 2.5) | m), 3.30-4.32 (7H, m), 3.05 (1H, |
| | 5,6,7,8- | ylcarbamate | | dd), 2.65-2.90 (2H, m), |
| | tetrahydroquinazolin- | | | 1.60-2.30 (4H, m), 1.47 (9H, s) |
| | 4- | | | |
| | yl]pyrrolidin-3- | | | |
| | yl}carbamate | | | |

### EXAMPLE 6

### Synthesis of 2-amino-7-(4-fluorophenyl)-5,6,7,8-tetrahydroquinazolin-4-ol (Intermediate 29)

3-(4-Fluorophenyl)cyclohexanone (CAS RN 78494-26-5) (1.44 g) is dissolved in dry THF (12 ml) under N₂ and NaH 60% dispersion in oil (319 mg) added in one portion. After 20 mins, dimethyl carbonate (0.759 ml) is added, and the mixture heated with stirring at 70° C. for 18 hrs. The THF is then removed in vacuo, the residue dissolved in DCM (60 ml) and washed with brine (20 ml) diluted with water (10 ml). The organic phase is separated, dried (MgSO₄) and concentrated in vacuo. Flash chromatography of the residue, eluting with DCM-Heptane 1:1, then 5:2 affords the crude beta-keto ester (809 mg, 43%) as a yellow oil. This is redissolved in abs. EtOH (15 ml), guanidine carbonate (582 mg) added and the mixture heated with stirring at 75° C. for 18 hrs. The EtOH is removed in vacuo, and the residue treated with DCM (25 ml) and water (10 ml). The undissolved solids are filtered off, washed with DCM (5 ml) and dried under vacuum to afford the title compound as a cream solid (361 mg, 43%). LCMS 260 [M+H]⁺, RT 1.91 mins (pH 2.5). ¹H NMR 300 MHz (d₆-DMSO) (δ ppm): 7.30 (2H, m), 7.10 (2H, t), 6.43 (2H, bs), 3.33 (3H+H₂O, bs), 2.90 (1H, m), 2.40 (1H, m), 2.20 (1H, m), 1.89 (1H, m), 1.70 (1H, m).

Intermediate 30 is prepared in a similar manner to the method described for Intermediate 6 in Example 4.

### Synthesis of 4-chloro-7-(4-fluorophenyl)-5,6,7,8-tetrahydroquinazolin-2-amine (Intermediate 30)

Starting from Intermediate 29. LCMS 278 [M+H]⁺, RT 3.61 mins (pH 2.5). ¹H NMR 300 MHz (CDCl₃) (δ ppm): 7.20 (2H, m), 7.02 (2H, t), 4.92 (2H, bs), 2.58-3.08 (5H, m), 2.17 (1H, m), 1.85 (1H, m).

Intermediate 31 is prepared in a similar manner to the method described for Intermediate 1 in Example 1.

### Synthesis of tert-butyl (4aR*,7aR*)-6-(2-amino-5,6,7,8-tetrahydroquinazolin-4-yl)octahydro-1H-pyrrolo[3,4-b]pyridine-1-carboxylate (Intermediate 31)

Starting from 4-chloro-5,6,7,8-tetrahydroquinazolin-2-amine (CAS RN 111896-77-6) and using tert-butyl (4aR*,7aR*)-octahydro-1H-pyrrolo[3,4-b]pyridine-1-carboxylate. LCMS 374 [M+H⁺] RT 2.98 mins (pH 5.8). ¹H NMR 300 MHz (CDCl₃) (δ ppm) 5.16-5.34 (1H, br s), 4.52-4.77 (2H, br s), 3.93-4.13 (1H, br s), 3.71-3.84 (1H, m), 3.52-3.72 (2H, m), 3.41 (1H, d, J=11.3 Hz), 2.73-2.86 (1H, m), 2.52-2.70 (3H, m), 2.08-2.26 (1H, m), 1.81-1.96 (2H, m), 1.56-1.80 (3H, m), 1.28-1.56 (11H, m)

Intermediate 32 is prepared in a similar manner to the method described for Intermediate 4 in Example 2.

### Synthesis of methyl 2-oxo-4-pyridin-2-ylcyclohexanecarboxylate (Intermediate 32)

Starting from 3-(2-piridinyl)cyclohexanone (CAS RN 110225-73-5). LCMS 234 [M+H⁺] RT 1.88 mins (pH 2.5). ¹H NMR 300 MHz (CDCl₃) 12.19 (0.5H, s(enol)), 8.54-8.59 (1H, m), 7.64 (1H, td), 7.12-7.20 (2H, m), 3.78 (0.5H, d), 3.78 (3H, s), 2.00-3.15 (6H, m), 1.70-1.86 (1H, m).

Intermediate 33 is prepared in a similar manner to the method described for Intermediate 5 in Example 3.

### Synthesis of 2-amino-7-pyridin-2-yl-5,6,7,8-tetrahydroquinazolin-4-ol (Intermediate 33)

Starting from Intermediate 32. LCMS 243 [M+H⁺] RT 1.75 mins (pH 2.5). ¹H NMR 300 MHz (d₆-DMSO) 10.71 (1H, bs), 8.52 (1H, bd), 7.73 (1H, td), 7.33 (1H, d), 7.22 (1H, dd), 6.24 (2H, bs), 2.98-3.11 (1H, m), 2.65-2.78 (1H, dd), 2.38-2.59)₂H, m), 2.17-2.31 (1H, m), 1.93-2.40 (1H, m), 1.64-1.80 (1H, m).

Intermediate 34 is prepared in a similar manner to the method described for Intermediate 6 in Example 4.

### Synthesis of 4-chloro-7-pyridin-2-yl-5,6,7,8-tetrahydroquinazolin-2-amine (Intermediate 34)

Starting from Intermediate 33. LCMS 361 [M+H⁺] RT 2.76 mins (pH 5.8). ¹H NMR 300 MHz (d₄-MeOH) 8.82 (1H, d), 8.59 (1H, t), 8.07 (1H, d), 7.98 (1H, t), 3.52-3.84 (3H, m), 2.90-3.01 (1H, m), 2.73-2.87 (1H, m), 2.34-2.45 (1H, m), 2.06 (1H, m).

Intermediates 35 and 36 are prepared in a similar manner to the method described for Intermediate 4 in Example 2. The reagents used and the results obtained are tabulated below (Table 5). The free base of the compounds is obtained unless otherwise stated.

TABLE 5

| | | | | ¹H NMR |
|---|---|---|---|---|
| | | Starting | | (Solvent, |
| Int. No | IUPAC Name | Materials | LCMS | δ ppm) |
| 35 | methyl 4-(5- | 3-(5- | 272 [M + H]⁺, | CDCl₃ 12.15 (1H, s), 6.74 (1H, d), |
| | chloro-2- | chlorothien-2- | RT 4.86 mins | 6.61 (1H, dd), 3.78 (3H, s), |
| | thienyl)-2- | yl)cyclohexanone | (pH 5.8) | 3.05-3.19 (1H, m), 2.25-2.70 (3H, mm), |
| | oxocyclohexanecarboxylate | (CAS RN | | 1.80-1.90 (1H, m), |
| | | 909421-72-3) | | 1.61-1.73 (2H, m). |
| 36 | methyl 2-(5- | 3-(5- | 272 [M + H]⁺, | CDCl₃ 12.52 (1H, s), 6.71 (1H, d), |
| | chloro-2- | chlorothien-2- | RT 4.73 mins | 6.48 (1H, dd), 3.66 (3H, s), |
| | thienyl)-6- | yl)cyclohexanone | (pH 5.8) | 4.04-4.08 (1H, m), 2.25-2.70 (3H, mm), |
| | oxocyclohexanecarboxylate | | | 1.80-1.90 (1H, m), |
| | | | | 1.61-1.73 (2H, m) |

Intermediate 37 is prepared in a similar manner to the method described for Intermediate 5 in Example 3.

### Synthesis of 2-amino-7-(5-chloro-2-thienyl)-5,6,7,8-tetrahydroquinazolin-4-ol (Intermediate 37)

Starting from Intermediate 35. LCMS 282 [M+H⁺] RT 2.74 mins (pH 5.8). ¹H NMR 300 MHz (d₆-DMSO) 10.88 (1H, bs), 6.96 (1H, d), 6.8 (1H, d), 6.30 (2H, bs), 3.12-3.23 (1H, m), 2.69 (1H, d), 2.63 (1H, m), 2.00-2.12 (1H, m), 1.56-1.72 (1H, m).

Intermediate 38 is prepared in a similar manner to the method described for Intermediate 6 in Example 4.

### Synthesis of 4-chloro-7-(5-chloro-2-thienyl)-5,6,7,8-tetrahydroquinazolin-2-amine (Intermediate 38)

Starting from Intermediate 37. LCMS 300 [M+H⁺] RT 4.09 mins (pH 5.8). ¹H NMR 300 MHz (CDCl₃) 6.75 (1H, d), 6.61 (1H, dd), 4.97 (2H, bs), 3.19-3.30 (1H, m), 3.07 (1H, ddd), 2.76-2.89 (2H, m), 2.59-2.72 (1H, m), 2.23-2.33 (1H, m), 1.78-1.93 (1H, m).

### EXAMPLE 7

### Synthesis of 4-chloro-5-(5-chloro-2-thienyl)-5,6,7,8-tetrahydroquinazolin-2-amine (Intermediate 39)

Intermediate 36 (324 mg) is dissolved in abs. EtOH (15 ml), guanidine carbonate (324 mg) is added, and the mixture heated at 78° C. for 1.5 hrs. The EtOH is removed in vacuo, and the residual solid suspended in DCM (10 ml) and water (10 ml). The undissolved solids are collected by filtration, washed with water (5 ml) and dried in a vacuum oven at 40° C. The solid is partially dissolved in DCM-MeOH (approx. 5:1, 15 ml) and HCl in dioxane 4.0M (0.76 ml) is added, and the mixture sonicated with warming to achieve solution. The solvents are removed in vacuo and the residue is dried. Anhydrous dioxane (10 ml) is then added, followed by POCl₃ (20 ml) and the mixture is heated under N₂ at 110° C. for 3 hours. The volatiles are then removed in vacuo; the residue is quenched with ice/water and basified to approx. pH 14 with K₂CO₃. This is then extracted with EtOAc (150 ml), dried (MgSO₄) and concentrated in vacuo. The resultant solid is purified by HPLC at pH 5.8 to afford the title compound as a cream solid (50 mg, 14%). LCMS 300 [M+H⁺] RT 3.90 mins (pH 5.8). ¹H NMR 300 MHz (CDCl₃) 6.70 (1H, d), 6.36 (1H, dd), 5.02 (2H, bs), 4.39 (1H, bt), 2.80 (1H, dtd), 2.59-2.75 (1H, m), 1.90-2.11 (2H, m), 1.75-1.86 (2H, m).

Intermediate 40 is prepared in a similar manner to the method described for Intermediate 1 in Example 1.

### Synthesis of tert-butyl (4aR*,7aR*)-6-[2-amino-7-(4-fluorophenyl)-5,6,7,8-tetrahydroquinazolin-4-yl]octahydro-1H-pyrrolo[3,4-b]pyridine-1-carboxylate (Intermediate 40)

Starting from tert-butyl (4aR*,7aR*)-octahydro-1H-pyrrolo[3,4-b]pyridine-1-carboxylate and Intermediate 30. LCMS 468 [M+H]⁺, RT 2.80 mins (pH 2.5). ¹H NMR 300 MHz (CDCl₃) (δ ppm): 7.18 (2H, m), 7.00 (2H, m), 5.55 (2H, bs), 4.70 (1H, m), 4.03 (1H, bm), 3.41-3.90 (4H, m), 2.60-3.12 (6H, m), 1.58-2.28 (5H, m), 1.20-1.52 (11H, m).

Intermediates 41 and 42 are prepared in a similar manner to the method described for Intermediate 1 in Example 1. The reagents used and the results obtained are tabulated below (Table 6). The free base of the compounds is obtained unless otherwise stated.

TABLE 6

| | | | | ¹H NMR |
|---|---|---|---|---|
| | | Starting | | (Solvent, |
| Int. No | IUPAC Name | Materials | LCMS | δ ppm) |
| 41 | tert-butyl (2-{[2- | Intermediate | 416 [M + H]⁺, | CDCl₃ 7.18 (2H, m), 7.00 (2H, m), |
| | amino-7-(4- | 30, tert-butyl | RT 2.43 mins | 5.65 (2H, bm), 4.92 (1H, bm), |
| | fluorophenyl)- | N-methyl-N- | (pH | 3.28-3.61 (4H, m), |
| | 5,6,7,8- | (2- | 2.5) | 2.51-3.10 (7H, m), 2.35 (1H, m), 2.10 (1H, |
| | tetrahydroquinazolin- | aminoethyl)carbamate | | m), 1.82 (0.5H, m), 1.68 (0.5H, |
| | 4- | (CAS | | m), 1.45 (9H, s) |
| | yl]amino}ethyl)methylcarbamate | RN 121492- | | |
| | | 06-6) | | |
| 42 | tert-butyl [1-(2- | Intermediate | 348 [M + H]⁺, | CDCl₃ 6.55-6.70 (1H, m), |
| | amino-7,7- | 17, tert-butyl | RT 2.29 mins | 4.40-4.75 (5H, m), 4.15-4.35 (2H, m), |
| | dimethyl-5,6,7,8- | azetidin-3- | (pH | 2.35-2.50 (2H, m), |
| | tetrahydroquinazolin- | ylcarbamate | 2.5) | 1.65-1.75 (2H, m), 1.55-1.65 (2H, m), |
| | 4- | | | 1.45 (9H, s), 1.40 (6H, s) |
| | yl)azetidin-3- | | | |
| | yl]carbamate | | | |

### EXAMPLE 8

### Synthesis of 4-chloro-7-isobutyl-5,6,7,8-tetrahydroquinazolin-2-amine (Intermediate 43)

To a suspension of sodium hydride (402 mg) in THF (30 ml) under nitrogen is added 3-isobutylcyclohexanone (CAS No 5674-05-5) (1.55 g), and the mixture is stirred at room temperature for 30 mins. Dimethyl carbonate (2 ml) is added to the mixture, which is then heated to reflux for 5 hours. The solvents are removed in vacuo and the residue taken up in DCM and washed with brine, dried (MgSO₄) and concentrated in vacuo. The residue is then taken up in EtOH (30 ml), combined with guanidine carbonate (1.8 g) and heated to reflux for 3 hours to give a black solid (1.89 g). LCMS 222.3 [M+H]⁺, RT 2.66 mins (pH 5.8). The solid is taken up in MeOH (30 ml) and HCl in dioxan (15 ml) is added causing dissolution. The solvents are removed in vacuo and the residue, once dry, is diluted with dioxan (30 ml) and POCl₃ (30 ml) and the reaction mixture is heated to 110° C. for 3 hours. The solvents are removed in vacuo and the residue is taken up in EtOAc (150 ml), washed with K₂CO₃ aq (150 ml), dried (MgSO₄) and concentrated in vacuo to give a black solid (830 mg). LCMS 240 [M+H]⁺, RT 4.33 mins (pH 5.8). ¹H NMR 300 MHz (d₄-MeOH) (6 ppm): 2.38-2.48 (2H, m), 2.06 (1H, dd), 1.60-1.90 (3H, m), 1.00-1.25 (4H, m), 0.85 (6H, s).

Intermediate 44 is prepared in a similar manner to the method described for Intermediate 5 in Example 3.

### Synthesis of 2-amino-6,6-dimethyl-5,6,7,8-tetrahydroquinazolin-4-ol (Intermediate 44)

Starting from methyl 5,5-dimethyl-2-oxocyclohexanecarboxylate (CAS RN 50388-51-7). LCMS 194 [M+H]⁺, RT 1.51 mins (pH 2.5). ¹H NMR 300 MHz (d₆-DMSO) (δ ppm): 6.30 (2H, bs), 3.35 (1H+H₂O, bs), 2.30 (2H, m), 2.00 (2H, s), 1.41 (2H, m), 0.90 (6H, s).

Intermediate 45 is prepared in a similar manner to the method described for Intermediate 6 in Example 4.

### Synthesis of 4-chloro-6,6-dimethyl-5,6,7,8-tetrahydroquinazolin-2-amine (Intermediate 45)

Starting from Intermediate 44. LCMS 212 [M+H]⁺, RT 3.08 mins (pH 2.5). ¹H NMR 300 MHz (CDCl₃) (δ ppm): 4.93 (2H, bs), 2.70 (2H, t), 2.40 (2H, s), 1.60 (2H, t), 1.02 (6H, s).

Intermediates 46 to 48 are prepared in a similar manner to the method described for Intermediate 1 in Example 1. The reagents used and the results obtained are tabulated below (Table 7). The free base of the compounds is obtained unless otherwise stated.

TABLE 7

| | | | | ¹H NMR |
|---|---|---|---|---|
| | | Starting | | (Solvent, |
| Int. No | IUPAC Name | Materials | LCMS | δ ppm) |
| 46 | tert-butyl {2-[(2- | Intermediate | 350 [M + H]⁺, | CDCl₃ 3.52 (3H, bs), 2.90 (3H, s), |
| | amino-7,7- | 17, tert-butyl | RT 2.25 mins | 2.55 (2H, bm), 2.35 (2H, s), |
| | dimethyl-5,6,7,8- | N-methyl-N- | (pH | 2.22 (2H, t), 1.54 (2H, t), 1.46 (9H, s), |
| | tetrahydroquinazolin- | (2- | 2.5) | 1.30 (2H, m), 0.96 (6H, s) |
| | 4- | aminoethyl)carbamate | | |
| | yl)amino]ethyl}methylcarbamate | | | |
| 47 | tert-butyl 5-(2- | Intermediate | 388 [M + H]⁺, | CDCl₃ 4.86 (2H, bs), 3.86 (2H, |
| | amino-7,7- | 17, tert- | RT 2.38 mins | m), 3.50-3.66 (4H, m), 3.30 (2H, |
| | dimethyl-5,6,7,8- | butylhexahydropyrrolo[3, | (pH | m), 2.88 (2H, m), 2.65 (2H, t), |
| | tetrahydroquinazolin- | 4- | 2.5) | 2.38 (2H, s), 1.48 (2H, t), |
| | 4- | c]pyrrole-2- | | 1.45 (9H, s), 1.00 (6H, s) |
| | yl)hexahydropyrrolo[3, | (1H)- | | |
| | 4-c]pyrrole- | carboxylate | | |
| | 2(1H)- | (CAS RN | | |
| | carboxylate | 141449-85-6) | | |
| 48 | tert-butyl 4-(2- | Intermediate | 376 [M + H]⁺, | CDCl₃ 4.30 (2H, bm), 3.88 (2H, |
| | amino-6,6- | 45, tert-butyl | RT 2.46 mins | m), 3.68 (1H, m), 2.85-3.30 (4H, |
| | dimethyl-5,6,7,8- | 2- | (pH | m), 2.71 (2H, t), 2.22 (2H, q), |
| | tetrahydroquinazolin- | methylpiperazine- | 2.5) | 1.58 (2H, t), 1.48 (9H, s), 1.20 (3H, d), |
| | 4-yl)-2- | 1- | | 1.00 (3H, s), 0.92 (3H, s) |
| | methylpiperazine- | carboxylate | | |
| | 1-carboxylate | | | |

Intermediate 49 is prepared in a similar manner to the method described for Intermediate 5 in Example 3.

### Synthesis of 2-amino-8,8-dimethyl-5,6,7,8-tetrahydroquinazolin-4-ol (Intermediate 49)

Starting from methyl 3,3-dimethyl-2-oxocyclohexanecarboxylate (CAS RN 101327-97-3). LCMS 194 [M+H]⁺, RT 1.43 mins (pH 2.5). ¹H NMR 300 MHz (d₆-DMSO) (δ ppm): 6.25 (2H, bs), 3.35 (1H+H₂O, bs), 2.20 (2H, t), 1.47-1.65 (4H, m), 1.12 (6H, s).

Intermediate 50 is prepared in a similar manner to the method described for Intermediate 6 in Example 4.

### Synthesis of 4-chloro-8,8-dimethyl-5,6,7,8-tetrahydroquinazolin-2-amine (Intermediate 50)

Starting from Intermediate 49. LCMS 212 [M+H]⁺, RT 3.78 mins (pH 2.5). ¹H NMR 300 MHz (CDCl₃) (δ ppm): 5.25 (2H, bm), 2.60 (2H, t), 1.80 (2H, m), 1.70 (2H, m), 1.30 (6H, s).

Intermediates 51 to 55 are prepared in a similar manner to the method described for Intermediate 1 in Example 1. The reagents used and the results obtained are tabulated below (Table 8). The free base of the compounds is obtained unless otherwise stated.

TABLE 8

| | | | | ¹H NMR |
|---|---|---|---|---|
| | | Starting | | (Solvent, |
| Int. No | IUPAC Name | Materials | LCMS | δ ppm) |
| 51 | tert-butyl 4-(2- | Intermediate | 376 [M + H]⁺, | CDCl₃ 9.75 (2H, bs), 8.45 (1H, |
| | amino-8,8- | 50, tert-butyl | RT 2.40 mins | bs, HCOOH), 4.25-4.40 (1H, |
| | dimethyl-5,6,7,8- | 2- | (pH | bm), 4.10-4.20 (1H, m), |
| | tetrahydroquinazolin- | methylpiperazine- | 2.5) | 3.85-4.05 (2H, m), 3.05-3.45 (3H, m), |
| | 4-yl)-2- | 1- | | 2.35-2.55 (2H, m), |
| | methylpiperazine- | carboxylate | | 1.55-1.80 (4H, m), 1.50 (9H, s), 1.43 (3H, s), |
| | 1-carboxylate | | | 1.41 (3H, s), 1.20 (3H, d) |
| | formic acid salt | | | |
| 52 | tert-butyl [1-(2- | Intermediate | 348 [M + H]⁺, | CDCl₃ 8.55 (2H, bs), 7.65 (1H, |
| | amino-8,8- | 50, tert-butyl | RT 2.19 mins | bs, HCOOH), 6.55-6.70 (1H, m), |
| | dimethyl-5,6,7,8- | azetidin-3- | (pH | 4.40-4.75 (3H, m), |
| | tetrahydroquinazolin- | ylcarbamate | 2.5) | 4.15-4.35 (2H, m), 2.35-2.50 (2H, m), |
| | 4- | | | 1.65-1.75 (2H, m), 1.55-1.65 (2H, |
| | yl)azetidin-3- | | | m), 1.45 (9H, s), 1.40 (6H, s) |
| | yl]carbamate | | | |
| | formic acid salt | | | |
| 53 | tert-butyl | Intermediate | 402 [M + H]⁺, | CDCl₃ 4.65-4.80 (1H, m), |
| | (4aR*,7aR*)-6- | 50, tert-butyl | RT 2.52 mins | 3.95-4.10 (1H, m), 3.60-3.90 (3H, m), |
| | (2-amino-8,8- | (4aR*,7aR*)- | (pH | 3.55 (1H, d), 2.70-2.85 (1H, m), |
| | dimethyl-5,6,7,8- | octahydro-1H- | 2.5) | 2.55-2.65 (2H, m), |
| | tetrahydroquinazolin- | pyrrolo[3,4- | | 2.15-2.30 (1H, m), 1.65-1.85 (4H, m), |
| | 4- | b]pyridine-1- | | 1.55-1.65 (2H, m), 1.52 (3H, s), |
| | yl)octahydro-1H- | carboxylate | | 1.48 (12H, s), 1.20-1.40 (3H, m), |
| | pyrrolo[3,4- | | | 0.95 (1H, dd) |
| | b]pyridine-1- | | | |
| | carboxylate | | | |
| 54 | tert-butyl 4-[(2- | Intermediate | 376 [M + H]⁺, | CDCl₃ 8.70 (1H, bs, HCOOH), |
| | amino-8,8- | 50, tert-butyl | RT 2.21 mins | 5.00 (1H, d), 4.00-4.30 (4H, m), |
| | dimethyl-5,6,7,8- | 4- | (pH | 2.80-3.00 (2H, m), |
| | tetrahydroquinazolin- | aminopiperidine- | 2.5) | 2.10-2.25 (2H, m), 1.95-2.10 (2H, m), |
| | 4- | 1- | | 1.75-1.90 (2H, m), 1.60-1.70 (2H, |
| | yl)amino]piperidine- | carboxylate | | m), 1.50-1.60 (1H, m), 1.50 (9H, |
| | 1-carboxylate | (CAS RN | | s), 1.45 (6H, s), 1.25-1.45 (2H, |
| | formic acid salt | 87120-72-7) | | m) |
| 55 | tert-butyl 3-[(2- | Intermediate | 362 [M + H]⁺, | CDCl₃ 8.65 (1H, s, HCOOH), |
| | amino-8,8- | 50, tert-butyl | RT 2.17 mins | 5.20-5.30 (1H, m), |
| | dimethyl-5,6,7,8- | 3-amino-1- | (pH | 4.60-4.75 (2H, m), 3.65-3.80 (1H, m), |
| | tetrahydroquinazolin- | pyrrolidinecarboxylate | 2.5) | 3.40-3.60 (2H, m), 3.15-3.35 (1H, |
| | 4- | (CAS | | m), 2.25-2.35 (1H, m), |
| | yl)amino]pyrrolidine- | RN 186550- | | 2.15-2.25 (2H, t), 1.85-2.00 (1H, m), |
| | 1- | 13-0) | | 1.70-1.85 (2H, m), |
| | carboxylate | | | 1.60-1.70 (2H, m), 1.50-1.60 (1H, m), |
| | formic acid salt | | | 1.50 (9H, s), 1.45 (6H, s) |

Intermediate 56 is prepared in a similar manner to the method described for Intermediate 4 in Example 2.

### Synthesis of methyl 2-oxospiro[5.5]undecane-3-carboxylate (Intermediate 56)

Starting from spiro[5,5]undecan-2-one (CAS RN 1781-81-3). LCMS 225 [M+H]⁺, RT 4.91 mins (pH 2.5). ¹H NMR 300 MHz (CDCl₃) (δ ppm): 12.10 (1H, s), 3.75 (3H, s), 2.20 (1H, t), 2.10 (2H, s), 2.00-2.10 (1H, m), 1.10-1.60 (12H, m).

Intermediate 57 is prepared in a similar manner to the method described for Intermediate 5 in Example 3.

### Synthesis of 2′-amino-5′,8′-dihydro-6′H-spiro[cyclohexane-1,7′-quinazolin]-4′-ol (Intermediate 57)

Starting from Intermediate 56. LCMS 234 [M+H]⁺, RT 1.82 mins (pH 2.5). ¹H NMR 300 MHz (d₆-DMSO) (δ ppm): 10.90 (1H, bs), 6.25 (2H, bs), 2.18 (4H, m), 1.45 (8H, m), 1.25 (4H, s).

Intermediate 58 is prepared in a similar manner to the method described for Intermediate 6 in Example 4.

### Synthesis of 4′-chloro-5′,8′-dihydro-6′H-spiro[cyclohexane-1,7′-quinazolin]-2′-amine (Intermediate 58)

Starting from Intermediate 57. LCMS 252 [M+H]⁺, RT 3.90 mins (pH 2.5). ¹H NMR 300 MHz (CDCl₃) (δ ppm): 8.20 (2H, bm), 2.85 (2H, bs), 2.62 (2H, bs), 2.4 (4H, bm), 1.80 (2H, s), 1.35 (4H, m)

Intermediate 59 is prepared in a similar manner to the method described for Intermediate 5 in Example 3.

### Synthesis of tert-butyl 2-amino-4-hydroxy-5,8-dihydropyrido[3,4-d]pyrimidine-7(6H)-carboxylate (Intermediate 59)

Starting from 1,4-piperidinedicarboxylic acid, 3-oxo-, 1-(1,1-dimethylethyl) 4-methyl ester (CAS RN 220223-46-1). LCMS 266 [M+H]⁺, RT 1.83 mins (pH 2.5). ¹H NMR 300 MHz (d₆-DMSO) (δ ppm): 6.40 (2H, bs), 4.02 (2H, s), 3.40-3.51 (2H, m), 2.23-2.29 (2H, m), 1.43 (9H, s).

### EXAMPLE 9

### Synthesis of tert-butyl 2-[(2,2-dimethylpropanoyl)amino]-4-hydroxy-5,8-dihydropyrido[3,4-d]pyrimidine-7(6H)-carboxylate (Intermediate 60)

To a suspension of Intermediate 59 (468 mg), DMAP (5 mg), DIPEA (0.92 ml) in dry DMF (4 ml) under nitrogen is added trimethylacetic anhydride (1.07 ml). The mixture is stirred and heated at 70° C. for 21 hours. The DMF is removed in vacuo and the residue taken up in DCM (30 ml) and washed with aqueous NH₄Cl (20 ml), dried (MgSO₄) and concentrated in vacuo to give a brown oil. The oil is purified by flash chromatography, eluting with EtOAc-Heptane 1:1 to afford the title compound as a buff coloured glass (632 mg, 100%). R_{f} (EtOAc-Heptane 1:1) 0.32. LCMS 351 [M+H]⁺, RT 3.23 mins (pH 2.5). ¹H NMR 300 MHz (CDCl₃) (δ ppm): 11.85 (1H, s), 7.94 (1H, s), 4.25 (2H, s), 3.55-3.65 (2H, m), 2.50-2.62 (2H, m), 1.48 (9H, s), 1.30 (9H, s).

### EXAMPLE 10

### Synthesis of N-(4-hydroxy-5,6,7,8-tetrahydropyrido[3,4-d]pyrimidin-2-yl)-2,2-dimethylpropanamide (Intermediate 61)

Intermediate 60 (632 mg) dissolved in DCM (10 ml) is treated with TFA (3 ml) at room temperature for 3 hrs. The solution is concentrated in vacuo, the residue redissolved in DCM (40 ml) and washed with 1N NaOH (20 ml). The aqueous phase is separated, concentrated in vacuo and the solid residue extracted with DCM-MeOH 9:1 (3×300 ml). The combined extracts are concentrated in vacuo to afford the title compound as an orange crystalline solid (233 mg, 52%). LCMS 251 [M+H]⁺, RT 1.18 mins (pH 2.5). ¹H NMR 300 MHz (d₄-MeOH) (δ ppm): 3.69 (2H, s), 3.08 (2H, t), 2.53 (2H, t).

Intermediate 62 is prepared in a similar manner to the method described for Intermediate 1 in Example 1.

### Synthesis of N-[7-(5-cyanopyridin-2-yl)-4-hydroxy-5,6,7,8-tetrahydropyrido[3,4-d]pyrimidin-2-yl]-2,2-dimethylpropanamide (Intermediate 62)

Starting from 6-chloronicotinonitrile (CAS RN 33252-28-7). R_{f} (EtOAc-Heptane 3:1) 0.41. LCMS 353 [M+H]⁺, RT 2.99 mins (pH 2.5). ¹H NMR 300 MHz (CDCl₃) (δ ppm): 11.90 (1H, br s), 8.45 (1H, d), 7.98 (1H, s), 7.67 (1H, dd), 6.66 (1H, d), 4.45 (2H, s), 3.90 (2H, t), 2.70 (2H, t), 1.32 (9H, s).

### EXAMPLE 11

### Synthesis of N-[4-chloro-7-(5-cyanopyridin-2-yl)-5,6,7,8-tetrahydropyrido[3,4-d]pyrimidin-2-yl]-2,2-dimethylpropanamide (Intermediate 63)

Intermediate 62 (141 mg) is suspended in POCl₃ (3 ml) and heated at 100° C. for 60 mins. The POCl₃ is removed in vacuo, and the residue carefully quenched with ice. The mixture is basified to pH 14 with 48% NaOH solution and extracted with DCM (2×20 ml), dried (MgSO₄) and concentrated in vacuo. The residue is purified by flash chromatography, eluting with EtOAc-Heptane 1:1 to afford the title compound as a pale cream solid (39 mg, 26%). R_{f} (EtOAc-Heptane 1:2) 0.07. LCMS 371 [M+H]⁺, RT 3.40 mins (pH 2.5). ¹H NMR 300 MHz (CDCl₃) (δ ppm): 8.45 (1H, d), 8.03 (1H, s), 7.69 (1H, dd), 6.69 (1H, d), 4.80 (2H, s), 4.10 (2H, t), 2.92 (2H, t), 1.34 (9H, s).

Intermediates 64 and 65 are prepared in a similar manner to the method described for Intermediate 1 in Example 1. The reagents used and the results obtained are tabulated below (Table 9). The free base of the compounds is obtained unless otherwise stated.

TABLE 9

| | | | | 1H NMR |
|---|---|---|---|---|
| | | Starting | | (Solvent, |
| Int. No | IUPAC Name | Materials | LCMS | δ ppm) |
| 64 | N-[7-(5- | Intermediate | 435 [M + H]⁺, | CDCl₃ 8.45 (1H, d), 7.81 (1H, s), |
| | cyanopyridin-2- | 63, N-methyl | RT 1.69 mins | 7.69 (1H, dd), 6.62 (1H, d), |
| | yl)-4-(4- | piperazine | (pH | 4.59 (2H, s), 3.97 (2H, m), 3.55 (4H, |
| | methylpiperazin- | | 2.5) | m), 2.72 (2H, m), 2.52 (4H, m), |
| | 1-yl)-5,6,7,8- | | | 2.35 (3H, s), 1.35 (9H, s) |
| | tetrahydropyrido[3, | | | |
| | 4-d]pyrimidin- | | | |
| | 2-yl]-2,2- | | | |
| | dimethylpropanamide | | | |
| 65 | N-{4-hydroxy-7- | Intermediate | 397 [M + H]⁺, | CDCl₃ 11.88 (1H, br s), 8.55 (1H, |
| | [4- | 61, 2-chloro- | RT 3.67 mins | d), 8.01 (1H, s), 6.82 (1H, d), |
| | (trifluoromethyl)pyrimidin- | 4- | (pH | 4.63 (2H, s), 4.09 (2H, t), 2.68 (2H, m), |
| | 2-yl]- | trifluoromethyl | 2.5) | 1.33 (9H, s) |
| | 5,6,7,8- | pyrimidine | | |
| | tetrahydropyrido[3, | (CAS RN | | |
| | 4-d]pyrimidin- | 33034-67-2) | | |
| | 2-yl}-2,2- | | | |
| | dimethylpropanamide | | | |

Intermediate 66 is prepared in a similar manner to the method described for Intermediate 63 in Example 11.

### Synthesis of N-{4-chloro-7-[4-(trifluoromethyl)pyrimidin-2-yl]-5,6,7,8-tetrahydropyrido[3,4-d]pyrimidin-2-yl}-2,2-dimethylbropanamide (Intermediate 66)

Starting from Intermediate 65. LCMS 415/417 [M+H]⁺, RT 4.09 mins (pH 2.5). ¹H NMR 300 MHz (CDCl₃) (δ ppm): 8.55 (1H, d), 8.02 (1H, s), 6.85 (1H, d), 5.07 (2H, s), 4.20 (2H, m), 2.90 (2H, m), 1.33 (9H, s).

Intermediates 67 to 70 are prepared in a similar manner to the method described for Intermediate 1 in Example 1. The reagents used and the results obtained are tabulated below (Table 10). The free base of the compounds is obtained unless otherwise stated.

TABLE 10

| | | | | ¹H NMR |
|---|---|---|---|---|
| | | Starting | | (Solvent, |
| Int. No | IUPAC Name | Materials | LCMS | δ ppm) |
| 67 | 2,2-dimethyl-N- | Intermediate | 479 [M + H]⁺, | CDCl₃ 8.53 (1H, d), 7.83 (1H, br |
| | {4-(4- | 66, N-methyl | RT 2.01 mins | s), 6.80 (1H, d), 4.88 (2H, s), |
| | methylpiperazin- | piperazine | (pH | 4.03 (2H, t), 3.55 (4H, m), 2.74 (2H, t), |
| | 1-yl)-7-[4- | | 2.5) | 2.51 (4H, m), 2.32 (3H, s), |
| | (trifluoromethyl)pyrimidin- | | | 1.30 (9H, s) |
| | 2-yl]- | | | |
| | 5,6,7,8- | | | |
| | tetrahydropyrido[3, | | | |
| | 4-d]pyrimidin- | | | |
| | 2- | | | |
| | yl}propanamide | | | |
| 68 | tert-butyl | Intermediate | 442 [M + H]⁺, | CDCl₃ 6.60 (2H, br s), 4.75 (1H, |
| | (4aR*,7aR*)-6- | 58, tert-butyl | RT 2.68 mins | m), 4.05 (1H, m), 3.80 (2H, m), |
| | (2′-amino-5′,8′- | (4aR*,7aR*)- | (pH | 3.65 (2H, m), 2.60-2.85 (4H, m), |
| | dihydro-6′H- | octahydro-1H- | 2.5) | 2.20 (1H, m), 1.70-1.83 (4H, m), |
| | spiro[cyclohexane- | pyrrolo[3,4- | | 1.50 (9H, s), 1.25-1.55 (13H, m) |
| | 1,7′- | b]pyridine-1- | | |
| | quinazolin]-4′- | carboxylate | | |
| | yl)octahydro-1H- | | | |
| | pyrrolo[3,4- | | | |
| | b]pyridine-1- | | | |
| | carboxylate | | | |
| 69 | tert-butyl [1-(2′- | Intermediate | 402 [M + H]⁺, | CDCl₃ 8.60 (1H, s), 4.88 (1H, br |
| | amino-5′,8′- | 58, tert-butyl | RT 2.58 mins | s), 4.55 (2H, br s), 4.35 (1H, br s), |
| | dihydro-6′H- | azetidin-3- | (pH | 2.93 (3H, s), 2.43 (4H, m), |
| | spiro[cyclohexane- | ylmethylcarbamate | 2.5) | 1.55 (2H, m), 1.45 (9H, s), |
| | 1,7′- | (CAS | | 1.20-1.50 (12H, m), 0.90 (2H, t) |
| | quinazolin]-4′- | RN 577777- | | |
| | yl)azetidin-3- | 20-9) | | |
| | yl]methylcarbamate | | | |
| | formic acid | | | |
| | salt | | | |
| 70 | tert-butyl [1-(2- | Intermediate | 390 [M + H]⁺, | d₄-MeOH 3.55-4.20 (4H, bm), |
| | amino-7- | 43, tert-butyl | RT 3.60 mins | 2.65-2.90 (3H, m), |
| | isobutyl-5,6,7,8- | pyrrolidin-3- | (pH | 1.70-2.30 (7H, bm), 1.50 (9H, s), |
| | tetrahydroquinazolin- | ylcarbamate | 5.8) | 1.15-1.55 (3H, m), 0.95 (6H, d) |
| | 4- | | | |
| | yl)pyrrolidin-3- | | | |
| | yl]carbamate | | | |
| | acetic acid salt | | | |

Intermediate 71 is prepared in a similar manner to the method described for Intermediate 4 in Example 2.

### Synthesis of methyl 4-isopropyl-2-oxocyclohexanecarboxylate (Intermediate 71)

Starting from 3-isopropylcyclohexanone (CAS RN 23396-36-3). LCMS [M+H]⁺ (not seen), RT 4.64 mins (pH 2.5). ¹H NMR 300 MHz (CDCl₃) (δ ppm): 12.10 (1H, s), 3.75 (3H, s), 2.30 (2H, m), 1.30-2.20 (6H, m), 0.90 (6H, m).

Intermediate 72 is prepared in a similar manner to the method described for Intermediate 5 in Example 3.

### Synthesis of 2-amino-7-isopropyl-5,6,7,8-tetrahydroquinazolin-4-ol (Intermediate 72)

Starting from Intermediate 71. LCMS 208 [M+H]⁺, RT 1.65 mins (pH 2.5). ¹H NMR 300 MHz (d₆-DMSO) (δ ppm): 6.20 (2H, bs), 1.00-2.48 (8H, m), 0.92 (6H, d).

Intermediate 73 is prepared in a similar manner to the method described for Intermediate 6 in Example 4.

### Synthesis of 4-chloro-7-isopropyl-5,6,7,8-tetrahydroquinazolin-2-amine (Intermediate 73)

Starting from Intermediate 72. LCMS 226 [M+H]⁺, RT 3.55 mins (pH 2.5). ¹H NMR 300 MHz (CDCl₃) (δ ppm): 5.72 (2H, bs), 2.70-3.05 (2H, m), 2.30-2.65 (2H, m), 1.82-2.30 (1H, m), 1.15-1.75 (3H, m), 0.92 (6H, d).

Intermediates 74 and 75 are prepared in a similar manner to the method described for Intermediate 1 in Example 1. The reagents used and the results obtained are tabulated below (Table 11). The free base of the compounds is obtained unless otherwise stated.

TABLE 11

| | | | | ¹H NMR |
|---|---|---|---|---|
| | | Starting | | (Solvent, |
| Int. No | IUPAC Name | Materials | LCMS | δ ppm) |
| 74 | tert-butyl | Intermediate | 416 [M + H]⁺, | CDCl₃ 8.52 (1H, s), |
| | (4aR*,7aR*)-6- | 73, tert-butyl | RT 2.68 mins | 4.65-4.80 (1H, m), 3.45-4.10 (5H, m), |
| | (2-amino-7- | (4aR*,7aR*)- | (pH | 1.00-2.90 (15H, m), 1.48 (9H, s), |
| | isopropyl- | octahydro-1H- | 2.5) | 0.80-1.00 (6H, m) |
| | 5,6,7,8- | pyrrolo[3,4- | | |
| | tetrahydroquinazolin- | b]pyridine-1- | | |
| | 4- | carboxylate | | |
| | yl)octahydro-1H- | | | |
| | pyrrolo[3,4- | | | |
| | b]pyridine-1- | | | |
| | carboxylate | | | |
| | formic acid salt | | | |
| 75 | tert-butyl [1-(2- | Intermediate | 376 [M + H]⁺, | CDCl₃ 8.63 (1H, s), |
| | amino-7- | 73, tert-butyl | RT 2.47 mins | 4.70-5.00 (2H, bs), 4.22-4.61 (5H, m), |
| | isopropyl- | azetidin-3- | (pH | 2.95 (3H, s), 2.52-2.78 (2H, m), |
| | 5,6,7,8- | ylmethylcarbamate | 2.5) | 2.30-2.50 (2H, m), |
| | tetrahydroquinazolin- | | | 1.89-2.01 (1H, m), 1.55-1.71 (1H, m), |
| | 4- | | | 1.46 (9H, s), 1.39-1.48 (1H, m), |
| | yl)azetidin-3- | | | 1.15-1.35 (1H, m), 0.88-0.99 (6H, |
| | yl]methylcarbamate | | | m). |
| | formic acid | | | |
| | salt | | | |

Compound 1 is prepared in a similar manner to the method described for Intermediate 1 in Example 1.

### Synthesis of 4-(4-methylpiperazin-1-yl)-6,7-dihydro-5H-cyclopenta[d]pyrimidin-2-amine (Compound 1)

Starting from 4-chloro-6,7-dihydro-5H-cyclopenta[d]pyrimidin-2-amine and using N-methyl piperazine. LCMS 234 [M+H]⁺, RT 1.68 mins (pH 5.8). ¹H NMR 300 MHz (d₄-MeOH) (δ ppm): 3.73 (4H, m), 2.92 (2H, t), 2.69 (2H, t), 2.50 (4H, m), 2.34 (3H, s), 2.03 (2H, m).

### EXAMPLE 12

### Synthesis of 4-(3-aminopyrrolidin-1-yl)-6,7-dihydro-5H-cyclopenta[d]pyrimidin-2-amine (Compound 2)

Intermediate 1 (93 mg) is dissolved in DCM (6 ml), TFA (1 ml) added, and the solution allowed to stand at room temperature for 6 hrs. The solution is evaporated in vacuo, the residue redissolved in EtOAc (30 ml), and washed with sat. aqueous. NaHCO₃ (5 ml), 48% NaOH (0.25 ml) and brine (10 ml). The organic phase is separated, the aqueous back-extracted with EtOAc (20 ml), the combined organics dried (MgSO₄) and concentrated in vacuo to afford the title compound as a colourless glass (40 mg, 57%). LCMS 220 [M+H]⁺, RT 1.08 mins (pH 5.8). ¹H NMR 300 MHz (d₄-MeOH) (δ ppm): 3.62-4.00 (5H, m), 3.10 (2H, m), 2.72 (2H, m), 2.29 (1H, m), 1.90-2.12 (3H, m).

Compound 3 is prepared in a similar manner to the method described for Compound 2 in Example 12.

### Synthesis of 4-(3-aminopyrrolidin-1-yl)-6,7,8,9-tetrahydro-5H-cyclohepta[d]pyrimidin-2-amine (Compound 3)

Starting from Intermediate 2. LCMS 248 [M+H]⁺, RT 1.50 mins (pH 5.8). ¹H NMR 300 MHz (CDCl₃) (δ ppm): 4.67 (2H, bm), 3.70 (2H, m), 3.57 (2H, m), 3.25 (1H, dd), 2.72 (2H, m), 2.64 (2H, m), 2.07 (1H, m), 1.50-1.95 (8H, m).

Compounds 4 and 5 are prepared in a similar manner to the method described for Intermediate 1 in Example 1. The reagents used and the results obtained are tabulated below (Table 12). The free base of the compounds is obtained unless otherwise stated.

TABLE 12

| | | | | ¹H NMR |
|---|---|---|---|---|
| Comp. | | | | (Solvent, |
| No | IUPAC Name | Starting Materials | LCMS | δ ppm) |
| 4 | 4-(4- | 4-chloro-6,7,8,9- | 262 [M + H]⁺, | CDCl₃ 4.60 (2H, bm), |
| | methylpiperazin-1- | tetrahydro-5H- | RT 2.02 mins | 3.21 (4H, m), 2.73 (2H, m), |
| | yl)-6,7,8,9- | cyclohepta[d]pyrimidin- | (pH 5.8) | 2.45-2.62 (6H, m), 2.33 (3H, s), |
| | tetrahydro-5- | 2-amine, N- | | 1.83 (2H, m), |
| | cyclohepta[d]pyrimidin- | methylpiperazine | | 1.55-1.73 (4H, m) |
| | 2-amine | | | |
| 5 | 4-(4- | 4-chloro-6- | 324 [M + H]⁺, | CDCl₃ 7.20-7.40 (5H, m), |
| | methylpiperazin-1- | phenyl-5,6,7,8- | RT 2.48 mins | 4.67 (2H, bs), |
| | yl)-6-phenyl- | tetrahydroquinazolin- | (pH 5.8) | 3.37-3.50 (2H, m), 3.17-3.30 (2H, |
| | 5,6,7,8- | 2-amine, N- | | m), 2.64-2.93 (4H, m), |
| | tetrahydroquinazolin- | methylpiperazine | | 2.33-2.60 (5H, m), |
| | 2-amine | | | 2.30 (3H, s), 1.92-2.20 (2H, m). |

*Comp. No means Compound Number

Compound 6 is prepared in a similar manner to the method described for Compound 2 in Example 12.

### Synthesis of 4-(3-aminopyrrolidin-1-yl)-6-phenyl-5,6,7,8-tetrahydroquinazolin-2-amine (Compound 6)

Starting from Intermediate 3. LCMS 310 [M+H]⁺, RT 1.92 mins (pH 5.8). ¹H NMR 300 MHz (CDCl₃) (δ ppm): 7.16-7.40 (5H, m), 5.70 (2H, bd), 3.30-4.00 (7H, m), 2.62-3.00 (5H, m), 1.53-2.20 (4H, m).

Compound 7 is prepared in a similar manner to the method described for Intermediate 1 in Example 1.

### Synthesis of 4-(4-methylpiperazin-1-yl)-7-phenyl-5,6,7,8-tetrahydroquinazolin-2-amine (Compound 7)

Starting from Intermediate 6 and using N-methylpiperazine. LCMS 324 [M+H]⁺, RT 2.53 mins (pH 5.8). ¹H NMR 300 MHz (CDCl₃) (δ ppm): 7.18-7.38 (5H, m), 4.85 (2H, bs), 3.45 (2H, m), 3.29 (2H, m), 3.10 (1H, m), 3.00 (1H, dd), 2.78 (1H, dd), 2.40-2.63 (6H, m), 2.34 (3H, s), 2.10 (1H, m), 1.74 (1H, m).

Compounds 8-11 are prepared in a similar manner to the method described for Compound 2 in Example 12. The reagents used and the results obtained are tabulated below (Table 13). The free base of the compounds is obtained unless otherwise stated.

TABLE 13

| | | | | ¹H NMR |
|---|---|---|---|---|
| Comp. | | Starting | | (Solvent, |
| No | IUPAC Name | Materials | LCMS | δ ppm) |
| 8 | 4-(3-aminoazetidin- | Intermediate 7 | 296 [M + H]⁺, | CDCl₃ 7.15-7.40 (5H, m), |
| | 1-yl)-7-phenyl- | | RT 2.12 mins | 5.20 (2H, bs), 4.49 (1H, dd), |
| | 5,6,7,8- | | (pH 5.8) | 4.39 (1H, m), |
| | tetrahydroquinazolin- | | | 3.75-3.95 (3H, m), 2.43-3.05 (7H, m), |
| | 2-amine | | | 2.10 (1H, m), 1.80 (1H, m) |
| 9 | 4-(3- | Intermediate 8 | 310 [M + H]⁺, | CDCl₃ 7.20-7.38 (5H, m), |
| | aminopyrrolidin-1- | | RT 1.91 mins | 5.20 (2H, bd), |
| | yl)-7-phenyl- | | (pH 5.8) | 3.50-4.00 (4H, m), 3.36 (1H, m), |
| | 5,6,7,8- | | | 2.29-3.05 (2H, m), |
| | tetrahydroquinazolin- | | | 2.66-2.85 (3H, m), 2.50 (2H, bm), |
| | 2-amine | | | 1.95-2.20 (2H, m), |
| | | | | 1.60-1.83 (2H, m) |
| 10 | 4-[(4aR*,7aR*)- | Intermediate 9 | 350 [M + H]⁺, | CDCl₃ 7.14-7.39 (5H, m), |
| | octahydro-6H- | | RT 2.19 mins | 4.90 (2H, bs), 3.93 (1H, t), |
| | pyrrolo[3,4- | | (pH 5.8) | 3.83 (1H, dd), 3.54 (1H, t), |
| | b]pyridin-6-yl]-7- | | | 3.26-3.45 (2H, m), |
| | phenyl-5,6,7,8- | | | 2.55-3.15 (7H, m), |
| | tetrahydroquinazolin- | | | 2.00-2.34 (4H, m), 1.40-1.93 (4H, m). |
| | 2-amine | | | |
| 11 | 4-(3- | Intermediate | 324 [M + H]⁺, | CDCl₃ 7.20-7.39 (5H, m), |
| | methylpiperazin-1- | 10 | RT 2.28 mins | 5.05 (2H, bs), 3.85 (1H, m), |
| | yl)-7-phenyl- | | (pH 5.8) | 3.71 (1H, m), |
| | 5,6,7,8- | | | 2.40-3.15 (11H, m), 2.11 (1H, m), |
| | tetrahydroquinazolin- | | | 1.75 (1H, m), 1.11 (3H, d) |
| | 2-amine | | | |

### EXAMPLE 13

### Synthesis of 4-(1,7-diazaspiro[4.4]non-7-yl)-7-phenyl-5,6,7,8-tetrahydroquinazolin-2-amine (Compound 12)

Intermediate 11 (62 mg) is dissolved in absolute EtOH (10 ml), HCl in diethyl ether 2.0M (0.071 ml) is added, the solution is degassed and flushed with N₂, 10% palladium on carbon (17 mg) is added and the reaction hydrogenated under 1 atm. H₂ at room temperature for 18 hrs. The catalyst is filtered off, and the filtrate concentrated in vacuo. The residue is redissolved in DCM (25 ml), washed with 1N NaOH solution (5 ml), dried (MgSO₄) and concentrated in vacuo to afford the title compound as a colourless solid (53 mg, 100%). LCMS 350 [M+H]⁺, RT 2.09 mins (pH 5.8). ¹H NMR 300 MHz (CDCl₃) (δ ppm): 7.15-7.39 (5H, m), 4.93 (2H, bd), 3.94 (1H, dt), 3.39-3.82 (5H, m), 2.63-3.14 (7H, m), 1.60-2.20 (7H, m)

Compound 13 is prepared in a similar manner to the method described for Intermediate 14 in Example 5.

### Synthesis of 7-(3-chlorophenyl)-4-(4-methylpiperazin-1-yl)-5,6,7,8-tetrahydroquinazolin-2-amine (Compound 13)

Starting from Intermediate 13 and using N-methylpiperazine. LCMS 358 [M+H]⁺, RT 2.58 mins (pH 5.8). ¹H NMR 300 MHz (CDCl₃) (δ ppm): 7.15 (2H, m), 7.02 (1H, s), 6.90 (1H, m), 4.75 (2H, bs), 4.00 (1H, m), 2.93-3.20 (4H, m), 2.72 (2H, m), 1.95-2.22 (8H, m), 1.60-1.80 (3H, m).

Compounds 14 and 15 are prepared in a similar manner to the method described for Compound 2 in Example 12. The reagents used and the results obtained are tabulated below (Table 14). The free base of the compounds is obtained unless otherwise stated.

TABLE 14

| | | | | ¹H NMR |
|---|---|---|---|---|
| Comp. | | Starting | | (Solvent, |
| No | IUPAC Name | Materials | LCMS | δ ppm) |
| 14 | 7-(3- | Intermediate | 358 [M + H]⁺, | CDCl₃ 7.30 (2H, m), |
| | chlorophenyl)-4- | 14 | RT 2.15 mins | 7.03 (1H, s), 6.91 (1H, m), |
| | (1,4-diazepan-1- | | (pH 5.8) | 5.00 (2H, bs), 4.05 (1H, m), |
| | yl)-5,6,7,8- | | | 3.20-3.55 (4H, m), |
| | tetrahydroquinazolin- | | | 2.68-3.05 (6H, m), 2.57 (1H, bs), |
| | 2-amine | | | 2.08 (1H, m), 1.50-1.83 (5H, m) |
| 15 | 7-(3- | Intermediate | 384 [M + H]⁺, | CDCl₃ 7.15 (2H, m), |
| | chlorophenyl)-4- | 15 | RT 2.27 | 7.05 (1H, s), 6.93 (0.5H, d), |
| | [(4aR*,7aR*)- | | and | 6.85 (0.5H, m), 5.82-6.55 (2H, |
| | octahydro-6H- | | 2.32 mins | bm), 4.39 (0.5H, m), |
| | pyrrolo[3,4- | | (pH 5.8) | 4.25 (0.5H, m), 3.09-3.75 (5H, |
| | b]pyridin-6-yl]- | | | m), 2.40-2.89 (4H, m), |
| | 5,6,7,8- | | | 1.10-2.23 (10H, m) |
| | tetrahydroquinazolin- | | | |
| | 2-amine | | | |

Compound 16 is prepared in a similar manner to the method described for Intermediate 1 in Example 1.

### Synthesis of 7,7-dimethyl-4-(4-methylpiperazin-1-yl)-5,6,7,8-tetrahydroquinazolin-2-amine (Compound 16)

Starting from Intermediate 17 and using N-methylpiperazine. LCMS 276 [M+H]⁺, RT 2.10 mins (pH 5.8). ¹H NMR 300 MHz (CDCl₃) (δ ppm): 5.53 (2H, bs), 3.44 (4H, m), 2.40-2.55 (8H, m), 2.33 (3H, s), 1.47 (2H, t), 1.02 (6H, s).

Compounds 17-21 are prepared in a similar manner to the method described for Compound 2 in Example 12. The reagents used and the results obtained are tabulated below (Table 15). The free base of the compounds is obtained unless otherwise stated.

TABLE 15

| | | | | ¹H NMR |
|---|---|---|---|---|
| Comp. | | Starting | | (Solvent, |
| No | IUPAC Name | Materials | LCMS | δ ppm) |
| 17 | 4-(1,4-diazepan-1-yl)- | Int. 18 | 276 [M + H]⁺, | CDCl₃ 6.00 (2H, bs), 5.50 (1H, |
| | 7,7-dimethyl-5,6,7,8- | | RT 2.02 mins | bs + H₂O), 3.75 (4H, m), |
| | tetrahydroquinazolin-2- | | (pH 5.8) | 3.09 (2H, t), 2.90 (2H, t), 2.55 (2H, t), |
| | amine | | | 2.45 (2H, s), 1.88 (2H, m), |
| | | | | 1.50 (2H, t), 1.03 (6H, s) |
| 18 | 4-(3-aminopyrrolidin-1- | Int. 19 | 262 [M + H]⁺, | CDCl₃ 5.45 (2H, bs), |
| | yl)-7,7-dimethyl- | | RT 1.79 mins | 3.80-3.90 (2H, m), 3.71 (1H, m), 3.62 (1H, |
| | 5,6,7,8- | | (pH 5.8) | m), 3.40 (1H, dd), 2.80 (2H, bm), |
| | tetrahydroquinazolin-2- | | | 2.69 (2H, t), 2.40 (2H, s), |
| | amine | | | 2.09 (1H, m), 1.71 (1H, m), 1.45 (2H, |
| | | | | t), 1.00 (6H, s) |
| 19 | 7,7-dimethyl-4- | Int. 20 | 302 [M + H]⁺, | CDCl₃ 5.55 (2H, bs), 3.90 (1H, |
| | [(4aR*,7aR*)- | | RT 1.19 mins | t), 3.78 (1H, dd), 3.67 (1H, t), |
| | octahydro-6H- | | (pH 2.5) | 3.56 (1H, d), 3.34 (1H, m), |
| | pyrrolo[3,4-b]pyridin-6- | | | 3.02 (1H, m), 2.58-2.83 (3H, m), |
| | yl]-5,6,7,8- | | | 2.40 (2H, s), 2.28 (1H, m), |
| | tetrahydroquinazolin-2- | | | 1.32-1.85 (6H, m), 1.02 (3H, s), |
| | amine | | | 0.97 (3H, s) |
| 20 | 4-(3-aminopyrrolidin-1- | Int. 21 | 344 [M + H]⁺, | CDCl₃ 7.18-7.32 (2H, m), |
| | yl)-7-(3-chlorophenyl)- | | RT 2.10 mins | 7.01 (1H, s), 6.90 (1H, m), 4.35 (1H, |
| | 5,6,7,8- | | (pH 5.8) | m), 3.28-3.93 (5H, m), |
| | tetrahydroquinazolin-2- | | | 2.65-2.90 (2H, m), 1.31-2.12 (9H, |
| | amine | | | m), 0.90 (1H, m) |
| 21 | 7,7-dimethyl-4-(3- | Int. 22 | LCMS | CDCl₃ 4.93 (2H, bs), 3.73 (2H, |
| | methylpiperazin-1-yl)- | | 276 [M + H]⁺, | m), 2.78-3.10 (4H, m), |
| | 5,6,7,8- | | RT 2.01 mins | 2.35-2.62 (6H, m), 1.48 (2H, t), |
| | tetrahydroquinazolin-2- | | (pH 5.8) | 1.10 (3H, d), 1.02 (6H, s) |
| | amine | | | |

*Int. means Intermediate

Compounds 22 and 23 are prepared in a similar manner to the method described for Intermediate 1 in Example 1. The reagents used and the results obtained are tabulated below (Table 16). The free base of the compounds is obtained unless otherwise stated.

TABLE 16

| | | | | ¹H NMR |
|---|---|---|---|---|
| Comp. | | Starting | | (Solvent, |
| No | IUPAC Name | Materials | LCMS | δ ppm) |
| 22 | 7,7-dimethyl-4- | Intermediate | 262 [M + H]⁺, | CDCl₃ 5.25 (2H, bs), |
| | piperazin-1-yl- | 17, piperazine | RT 1.71 mins | 3.50 (1H, bm), 3.33 (4H, m), |
| | 5,6,7,8- | | (pH 5.8) | 2.95 (4H, m), 2.46 (2H, t), |
| | tetrahydroquinazolin- | | | 2.42 (2H, s), 1.46 (2H, t), |
| | 2-amine | | | 1.00 (6H, s) |
| 23 | 7-(2-chlorophenyl)- | Intermediate | 358 [M + H]⁺, | CDCl₃ 7.39 (1H, d), |
| | 4-(4- | 25, N- | RT 2.71 mins | 7.13-7.30 (3H, m), 4.90 (2H, |
| | methylpiperazin-1- | methylpiperazine | (pH 5.8) | bs), 3.46-3.63 (3H, m), |
| | yl)-5,6,7,8- | | | 3.35 (2H, m), 3.10 (1H, |
| | tetrahydroquinazolin- | | | dd), 2.43-2.78 (7H, m), |
| | 2-amine | | | 2.35 (3H, s), 2.08 (1H, m), |
| | | | | 1.78 (1H, m) |

Compounds 24-26 are prepared in a similar manner to the method described for Compound 2 in Example 12. The reagents used and the results obtained are tabulated below (Table 17). The free base of the compounds is obtained unless otherwise stated.

TABLE 17

| | | | | ¹H NMR |
|---|---|---|---|---|
| Comp. | | Starting | | (Solvent, |
| No | IUPAC Name | Materials | LCMS | δ ppm) |
| 24 | 7-(2-chlorophenyl)- | Int. 26 | 358 [M + H]⁺, | CDCl₃ 7.38 (1H, d), |
| | 4-(1,4-diazepan-1- | | RT 2.37 mins | 7.11-7.30 (3H, m), 5.55 (2H, |
| | yl)-5,6,7,8- | | (pH 5.8) | bs), 4.50 (1H + H₂O, bs), |
| | tetrahydroquinazolin- | | | 3.60-3.83 (4H, m), |
| | 2-amine | | | 3.52 (1H, m), 2.85-3.25 (5H, |
| | | | | m), 2.50-2.78 (3H, m), |
| | | | | 1.70-2.13 (4H, m) |
| 25 | 7-(2-chlorophenyl)- | Int. 27 | 384 [M + H]⁺, | CDCl₃ 7.38 (1H, m), |
| | 4-[(4aR*,7aR*)- | | RT 2.36 mins | 7.10-7.30 (3H, m), |
| | octahydro-6H- | | (pH 5.8) | 3.75-4.50 (5H, m), |
| | pyrrolo[3,4-b]pyridin- | | | 3.30-3.67 (4H, m), 2.60-3.20 (7H, |
| | 6-yl]-5,6,7,8- | | | m), 1.70-2.20 (6H, m) |
| | tetrahydroquinazolin- | | | |
| | 2-amine | | | |
| 26 | 4-(3- | Int. 28 | 344 [M + H]⁺, | d₄-MeOH 8.48 (2H, |
| | aminopyrrolidin-1- | | RT 1.50 mins | HCOOH), 7.22-7.50 (4H, |
| | yl)-7-(2- | | (pH 2.5) | m), 3.90-4.22 (5H, m), |
| | chlorophenyl)- | | | 3.55 (1H, m), |
| | 5,6,7,8- | | | 2.90-3.05 (3H, m), 2.75 (1H, m), |
| | tetrahydroquinazolin- | | | 1.84-2.50 (4H, m) |
| | 2-amine bis formic | | | |
| | acid salt | | | |

Compounds 27 and 28 are prepared in a similar manner to the method described for Intermediate 1 in Example 1. The reagents used and the results obtained are tabulated below (Table 18). The free base of the compounds is obtained unless otherwise stated.

TABLE 18

| | | | | ¹H NMR |
|---|---|---|---|---|
| Comp. | | Starting | | (Solvent, |
| No | IUPAC Name | Materials | LCMS | δ ppm) |
| 27 | 4-(1,4-diazepan-1- | Intermediate | 342 [M + H]⁺, | CDCl₃ 7.20 (2H, m), |
| | yl)-7-(4- | 30, | RT 2.29 mins | 7.00 (2H, t), 4.60 (2H, m), |
| | fluorophenyl)- | homopiperazine | (pH 5.8) | 3.55-3.73 (4H, m), |
| | 5,6,7,8- | | | 2.50-3.15 (9H, m), 1.60-2.14 (5H, m) |
| | tetrahydroquinazolin- | | | |
| | 2-amine | | | |
| 28 | 7-(4-fluorophenyl)-4- | Intermediate | 342 [M + H]⁺, | CDCl₃ 7.20 (2H, m), |
| | (4-methylpiperazin- | 30, N- | RT 2.54 mins | 7.00 (2H, t), 5.02 (2H, bs), |
| | 1-yl)-5,6,7,8- | methylpiperazine | (pH 5.8) | 3.48 (2H, m), 3.32 (2H, m), |
| | tetrahydroquinazolin- | | | 2.93-3.10 (2H, m), |
| | 2-amine | | | 2.41-2.78 (7H, m), 2.34 (3H, s), |
| | | | | 2.07 (1H, m), 1.70 (1H, m) |

### EXAMPLE 14

### Synthesis of 4-(4-methylpiperazin-1-yl)-5,6,7,8-tetrahydroquinazolin-2-amine (Compound 29)

A solution of 100 mg of 4-chloro-5,6,7,8-tetrahydroquinazolin-2-amine (CAS RN 111896-77-6) in N-methylpiperazine (2 ml) is heated in a microwave for 1 hour at 180° C. The reaction mixture is concentrated and purified by preparative HPLC to give the title compound as a colourless solid (57.7 mg, 43%). LCMS 248 [M+H]⁺, RT 1.84 mins (pH 5.8). ¹H NMR 300 MHz (CDCl₃) 4.6 (2H, bs), 3.32-3.38 (4H, m), 2.65 (2h, t), 2.48-2.53 (4H, m), 2.45 (2H, t), 1.61-1.87 (4H, m)

### EXAMPLE 15

### Synthesis of 4-[(4aR*,7aR*)-octahydro-6H-pyrrolo[3,4-b]pyridin-6-yl]-5,6,7,8-tetrahydroquinazolin-2-amine (Compound 30)

Intermediate 31 is dissolved in dry DCM (2 ml), HCl in ether (0.25 ml, 2.0M) is added and the reaction mixture is stirred at room temperature for 16 hrs. The precipitate obtained is filtered off and washed with dichloromethane/methanol (1:1). The solid obtained is then dissolved in H₂O, the solution is made basic with NaOH solution (1.0M) and the aqueous layer is extracted with ethyl acetate. The organic layer is washed with H₂O, dried over MgSO₄ and evaporated in vacuo to afford the title compound (1.9 mg, 14%). LCMS 274 [M+H]⁺, RT 1.81 mins (pH 5.8). ¹H NMR 300 MHz (d₄-MeOH) 3.67-3.77 (2H, m), 3.53-3.62 (1H, m), 3.43-3.51 (1H, m), 3.17-3.25 (1H, m, partially obscured by d₄-MeOH), 2.78-2.92 (1H, m), 2.38-2.67 (5H, m), 2.15-2.30 (1H, m), 1.31-1.88 (8H, m).

Compounds 31-39 are prepared in a similar manner to the method described for Intermediate 1 in Example 1. The reagents used and the results obtained are tabulated below (Table 19). The free base of the compounds is obtained unless otherwise stated.

TABLE 19

| | | | | ¹H NMR |
|---|---|---|---|---|
| Comp. | | Starting | | (Solvent, |
| No | IUPAC Name | Materials | LCMS | δ ppm) |
| 31 | 4-piperazin-1-yl-7- | Intermediate | 311 [M + H⁺] | d₄-MeOH 8.51 (1H, m), |
| | pyridin-2-yl-5,6,7,8- | 34, | RT | 7.82 (1H, m), 7.41 (1H, bd), |
| | tetrahydroquinazolin- | piperazine | 1.63 mins | 7.31 (1H, m), 3.49 (1H, dd), |
| | 2-amine | | (pH 5.8) | 3.45 (1H, dd), 3.19-3.31 (3H, m), |
| | | | | 2.80-3.04 (6H, m), |
| | | | | 2.55-2.79 (2H, m), 2.10-2.21 (1H, |
| | | | | m), 1.84 (1H, m) |
| 32 | 4-(4- | Intermediate | 325 [M + H⁺] | d₄-MeOH 8.51 (1H, m), |
| | methylpiperazin-1- | 34, N- | RT | 7.82 (1H, m), 7.41 (1H, m), |
| | yl)-7-pyridin-2-yl- | methylpiperazine | 1.99 mins | 7.31 (1H, m), 3.53-3.64 (2H, m), |
| | 5,6,7,8- | | (pH 5.8) | 3.20-3.42 (3H, m), |
| | tetrahydroquinazolin- | | | 2.53-3.03 (8H, m), 2.40 (3H, s), |
| | 2-amine | | | 2.10-2.21 (1H, m), 1.84 (1H, m) |
| 33 | 4-(1,4-diazepan-1- | Intermediate | 325 [M + H⁺] | d₄-MeOH 8.51 (1H, bdd), |
| | yl)-7-pyridin-2-yl- | 34, | RT | 7.82 (1H, m), 7.41 (1H, bd), |
| | 5,6,7,8- | homopiperazine | 1.74 mins | 7.31 (1H, m), 3.66-3.9 (4H, m), |
| | tetrahydroquinazolin- | | (pH | 2.60-3.56 (9H, m), |
| | 2-amine | | 5.8) | 1.78-2.24 (4H, m) |
| 34 | 7-(5-chloro-2- | Intermediate | 350 [M + H⁺] | d₄-MeOH 6.83 (1H, d), |
| | thienyl)-4-piperazin- | 38, | RT | 6.76 (1H, dd), 3.20-3.50 (5H, m), |
| | 1-yl-5,6,7,8- | piperazine | 2.46 mins | 2.83-3.11 (5H, m), |
| | tetrahydroquinazolin- | | (pH | 2.57-2.76 (3H, m), 2.17-2.28 (1H, |
| | 2-amine | | 5.8) | bd), 1.63-1.78 (1H, m) |
| 35 | 5-(5-chloro-2- | Intermediate | 350 [M + H⁺] | d₄-MeOH 6.67 (1H, d), |
| | thienyl)-4-piperazin- | 39, | RT | 6.45 (1H, dd), 4.24 (1H, t), |
| | 1-yl-5,6,7,8- | piperazine | 2.27 mins | 3.10-3.30 (2H, m), 2.96-3.08 (2H, |
| | tetrahydroquinazolin- | | (pH | m), 2.51-2.74 (6H, m), |
| | 2-amine | | 5.8) | 1.91-2.02 (1H, m), 1.60-1.82 (3H, |
| | | | | m) |
| 36 | 5-(5-chloro-2- | Intermediate | 364 [M + H⁺] | d₄-MeOH 6.68 (1H, d), |
| | thienyl)-4-(4- | 39, N- | RT | 6.46 (1H, d), 4.24 (1H, t), |
| | methylpiperazin-1- | methylpiperazine | 2.71 mins | 3.05-3.30 (4H, m), 2.5-2.63 (2H, |
| | yl)-5,6,7,8- | | (pH | m), 2.10-2.35 (4H, m), |
| | tetrahydroquinazolin- | | 5.8) | 2.19 (3H, s), 1.90-2.08 (1H, m), |
| | 2-amine | | | 1.60-1.88 (3H, m) |
| 37 | 7-(5-chloro-2- | Intermediate | 364 [M + H⁺] | d₄-MeOH 6.83 (1H, d), |
| | thienyl)-4-(4- | 38, N- | RT | 6.75 (1H, dd), 3.44-3.56 (2H, m), |
| | methylpiperazin-1- | methylpiperazine | 2.95 mins | 3.25-3.38 (3H, m), 3.04 (1H, |
| | yl)-5,6,7,8- | | (pH | dd), 2.48-2.76 (7H, m), |
| | tetrahydroquinazolin | | 5.8) | 2.35 (3H, s), 2.16-2.27 (1H, m), |
| | 2-amine | | | 1.69 (1H, m) |
| 38 | 5-(5-chloro-2- | Intermediate | 364 [M + H⁺] | d₄-MeOH 6.80 (1H, d), |
| | thienyl)-4-(1,4- | 39, | RT | 6.58 (1H, d), 4.43 (1H, t), |
| | diazepan-1-yl)- | homopiperazine | 2.48 mins | 3.76-3.90 (1H, m), 3.51-3.70 (4H, |
| | 5,6,7,8- | | (pH | m), 3.0-3.42 (3H, m), |
| | tetrahydroquinazolin- | | 5.8) | 2.68 (2H, m), 2.05-2.20 (1H, m), |
| | 2-amine acetic acid | | | 1.82-2.04 (3H, m), |
| | salt | | | 1.7-1.82 (2H, m) |
| 39 | 7-(5-chloro-2- | Intermediate | 364 [M + H⁺] | d₄-MeOH 6.83 (1H, d), |
| | thienyl)-4-(1,4- | 38, | RT | 6.76 (1H, dd), 3.70-3.91 (4H, m), |
| | diazepan-1-yl)- | homopiperazine | 2.60 mins | 2.98-3.57 (6H, m), |
| | 5,6,7,8- | | (pH | 2.59-2.79 (3H, m), 2.0-2.29 (3H, |
| | tetrahydroquinazolin- | | 5.8) | m), 1.65-1.82 (1H, m) |
| | 2-amine bis acetic | | | |
| | acid salt | | | |

### EXAMPLE 16

### Synthesis of 7-(4-fluorophenyl)-4-[(4aR*,7aR*)-octahydro-6H-pyrrolo[3,4-b]pyridin-6-yl]-5,6,7,8-tetrahydroquinazolin-2-amine trifluoroacetic acid salt (Compound 40)

Intermediate 40 (50 mg) is dissolved in DCM (5 ml) and trifluoroacetic acid (2 ml) added, and the solution allowed to stand at room temperature for 3 hrs. Then the volatiles are removed in vacuo, the residue redissolved in DCM (20 ml) and washed with sat. NaHCO₃ (3 ml) plus 1N NaOH (0.25 ml). The organic phase is separated, dried (MgSO₄) and concentrated in vacuo to afford the title compound as a colourless glass (81 mg, 100%). LCMS 368 [M+H]⁺, RT 1.43 mins (pH 2.5). ¹H NMR 300 MHz (d₄-MeOH) (δ ppm): 7.20 (2H, m), 7.02 (2H, m), 3.70-4.60 (7H, m), 3.35 (1H, m), 2.55-3.10 (7H, m), 1.55-2.20 (6H, m), 1.30 (1H, m).

Compound 41 is prepared in a similar manner to the method described for Compound 2 in Example 12.

### Synthesis of 7-(4-fluorophenyl)-N-4-[2-(methylamino)ethyl]-5,6,7,8-tetrahydroquinazoline-2,4-diamine (Compound 41)

Starting from Intermediate 41. LCMS 316 [M+H]⁺, RT 1.89 mins (pH 5.8). ¹H NMR 300 MHz (CDCl₃) (δ ppm): 7.15 (2H, m), 7.00 (2H, m), 5.90 (2H, bs), 5.65 (1H, bm), 3.60 (2H, m), 2.63-3.00 (5H, m), 2.50 (3H, s), 2.36 (2H, m), 2.15 (1H, m), 1.85 (1H, m), 1.60 (1H, m).

### EXAMPLE 17

### Synthesis of 6-acetyl-4-(4-methylpiperazin-1-yl)-6,7-dihydro-5H-pyrrolo[3,4-d]pyrimidin-2-amine bis acetic acid salt (Compound 42)

A solution of 2,4-dichloro-5,7-dihydro-pyrrolo[3,4-d]pyrimidine-6-carboxylic acid tert-butyl ester (515 mg) (CAS RN 903129-71-5), N-methylpiperazine (0.2 ml) and DIPEA (0.65 ml) in NMP (5 ml) was heated under microwave irradiation at 150° C. for 30 mins. 4-Methoxybenzylamine (0.235 ml) was added and the resulting mixture was again heated under microwave irradiation at 150° C. for 30 mins. The reaction mixture was diluted with MTBE (400 ml) and washed with NaHCO₃ (3×100 ml) and brine (100 ml) and concentrated. After purification by flash chromatography, eluting with DCM-MeOH 95:5 the crude product was dissolved in MeOH (2 ml) and 2M HCl in Et₂O (5 ml). The reaction mixture was stirred for 18 hours, concentrated and redissolved in DCM (10 ml). After cooling to −78° C., DIPEA (0.5 ml) and acetyl chloride (0.18 ml) were added and the reaction mixture was stirred for 18 hours at room temperature. The reaction mixture was diluted with DCM (100 ml) and washed with NaHCO₃ (3×25 ml) and brine (25 ml) and concentrated. The crude product was dissolved in TFA (5 ml) and refluxed for 1 hour. The reaction mixture was concentrated and the product was purified by preparative HPLC to afford the title compound as a bis acetate salt (9.8 mg, 1.4%). LCMS 277 [M+H]⁺, RT 1.28 mins (pH 5.8). ¹H NMR 300 MHz (CDCl₃) (δ ppm): 4.68-4.79 (4H, m), 4.49 (2H, s), 3.65-3.71 (4H, m), 2.42-2.51 (4H, m), 2.30-2.35 (3H, m), 2.12-2.18 (3H, m).

Compound 43 is prepared in a similar manner to the method described for Compound 2 in Example 12.

### Synthesis of 4-(3-aminoazetidin-1-yl)-7,7-dimethyl-5,6,7,8-tetrahydroquinazolin-2-amine (Compound 43)

Starting from Intermediate 42. LCMS 248 [M+H]⁺, RT 1.69 mins (pH 5.8). ¹H NMR 300 MHz (CDCl₃) (δ ppm): 3.85-4.90 (5H, bm), 2.50 (2H, t), 2.44 (2H, s), 1.70 (4H, bm), 1.50 (2H, t), 1.00 (6H, s).

Compounds 44 and 45 are prepared in a similar manner to the method described for Intermediate 1 in Example 1. The reagents used and the results obtained are tabulated below (Table 20). The free base of the compounds is obtained unless otherwise stated.

TABLE 20

| | | | | ¹H NMR |
|---|---|---|---|---|
| Comp. | | Starting | | (Solvent, |
| No | IUPAC Name | Materials | LCMS | δ ppm) |
| 44 | 7-isobutyl-4- | Intermediate | 290 [M + H]⁺, | d₄-MeOH 3.30-3.42 (2H, m), |
| | piperazin-1-yl- | 43, piperazine | RT 2.23 mins | 3.08-3.25 (2H, m obscured by |
| | 5,6,7,8- | | (pH | MeOH peak), 2.76-2.97 (4H, |
| | tetrahydroquinazolin- | | 5.8) | mm), 2.63 (1H, dd), |
| | 2-amine | | | 2.36-2.44 (2H, m), 2.04 (1H, dd). |
| | | | | 1.61-1.86 (2H, mm), 0.98-1.26 (4H, |
| | | | | mm), 0.84 (6H, d) |
| 45 | 6,6-dimethyl-4- | Intermediate | 276 [M + H]⁺, | CDCl₃ 5.10 (2H, bs), 3.40 (4H, |
| | (4- | 45, N- | RT 1.91 mins | m), 2.69 (2H, t), 2.51 (4H, m), |
| | methylpiperazin- | methylpiperazine | (pH | 2.34 (3H, s), 2.20 (2H, s), |
| | 1-yl)-5,6,7,8- | | 5.8) | 1.58 (2H, t), 0.95 (6H, s) |
| | tetrahydroquinazolin- | | | |
| | 2-amine | | | |

Compounds 46 to 48 are prepared in a similar manner to the method described for Compound 2 in Example 12. The reagents used and the results obtained are tabulated below (Table 21). The free base of the compounds is obtained unless otherwise stated.

TABLE 21

| | | | | ¹H NMR |
|---|---|---|---|---|
| Comp. | | Starting | | (Solvent, |
| No | IUPAC Name | Materials | LCMS | δ ppm) |
| 46 | 7,7-dimethyl-N- | Intermediate | 250 [M + H]⁺, | CDCl₃ 6.70 (2H, bs), 6.04 (1H, |
| | 4-[2- | 46 | RT 1.55 mins | bm), 3.60 (2H, m), 3.25 (1H, bm), |
| | (methylamino)ethyl]- | | (pH | 2.93 (2H, t), 2.51 (3H, s), |
| | 5,6,7,8- | | 5.8) | 2.40 (2H, s), 2.25 (2H, t), 1.59 (2H, t), |
| | tetrahydroquinazoline- | | | 1.00 (6H, s) |
| | 2,4- | | | |
| | diamine | | | |
| 47 | 4- | Intermediate | 288 [M + H]⁺, | d₄-MeOH 3.96 (2H, dd), 3.85 (2H, |
| | (hexahydropyrrolo[3, | 47 | RT 1.97 mins | dd), 3.50 (2H, dd), 3.20 (2H, dd), |
| | 4-c]pyrrol- | | (pH | 3.11 (2H, m), 2.64 (2H, t), |
| | 2(1H)-yl)-7,7- | | 5.8) | 2.36 (2H, s), 1.45 (2H, t), 0.97 (6H, s) |
| | dimethyl-5,6,7,8- | | | |
| | tetrahydroquinazolin- | | | |
| | 2-amine | | | |
| 48 | 6,6-dimethyl-4- | Intermediate | 276 [M + H]⁺, | CDCl₃ 5.40 (2H, bs), 3.75 (3H, |
| | (3- | 48 | RT 2.16 mins | m), 2.80-3.09 (4H, m), 2.68 (2H, |
| | methylpiperazin- | | (pH | t), 2.52 (1H, dd), 2.20 (2H, s), |
| | 1-yl)-5,6,7,8- | | 5.8) | 1.55 (2H, t), 1.10 (3H, d), |
| | tetrahydroquinazolin- | | | 0.96 (3H, s), 0.94 (3H, s) |
| | 2-amine | | | |

Compounds 49 to 52 are prepared in a similar manner to the method described for Intermediate 1 in Example 1. The reagents used and the results obtained are tabulated below (Table 22). The free base of the compounds is obtained unless otherwise stated.

TABLE 22

| | | | | ¹H NMR |
|---|---|---|---|---|
| Comp. | | Starting | | (Solvent, |
| No | IUPAC Name | Materials | LCMS | δ ppm) |
| 49 | 4-(1,4-diazepan- | Intermediate | 276 [M + H]⁺, | CDCl₃ 6.47 (2H, bs), 4.25 (1H + AcOH, |
| | 1-yl)-6,6- | 45, | RT 1.94 mins | bs), 3.72 (4H, m), |
| | dimethyl-5,6,7,8- | homopiperazine | (pH | 3.10 (2H, m), 2.93 (2H, m), 2.71 (2H, |
| | tetrahydroquinazolin- | | 5.8) | t), 2.25 (3H, AcOH), 2.03 (2H, s), |
| | 2-amine | | | 1.90 (2H, m), 1.55 (2H, t), |
| | acetic acid salt | | | 0.95 (6H, s) |
| 50 | 6,6-dimethyl-4- | Intermediate | 262 [M + H]⁺, | CDCl₃ 9.85 (1H + AcOH, bs), |
| | piperazin-1-yl- | 45, piperazine | RT 1.97 mins | 6.85 (2H, bm), 3.64 (4H, m), |
| | 5,6,7,8- | | (pH | 3.15 (4H, m), 2.72 (2H, t), 2.17 (2H, s), |
| | tetrahydroquinazolin- | | 5.8) | 2.03 (6H, AcOH), 1.55 (2H, t), |
| | 2-amine bis | | | 0.94 (6H, s) |
| | acetic acid salt | | | |
| 51 | 8,8-dimethyl-4- | Intermediate | 276 [M + H]⁺, | CDCl₃ 4.70 (2H, s), 3.30 (4H, t), |
| | (4- | 50, N- | RT 2.07 mins | 2.55 (4H, t), 2.45 (2H, m), |
| | methylpiperazin- | methylpiperazine | (pH | 2.32 (3H, s), 1.65 (4H, d), 1.22 (6H, s) |
| | 1-yl)-5,6,7,8- | | 5.8) | |
| | tetrahydroquinazolin- | | | |
| | 2-amine | | | |
| 52 | 4-(1,4-diazepan- | Intermediate | 276 [M + H]⁺, | CDCl₃ 5.55 (3H, bs), 3.82 (2H, t), |
| | 1-yl)-8,8- | 50, | RT 1.90 mins | 3.68 (2H, t), 3.32 (2H, t), |
| | dimethyl-5,6,7,8- | homopiperazine | (pH | 3.10 (2H, t), 2.45 (2H, bs), 2.10 (2H, |
| | tetrahydroquinazolin- | | 5.8) | m), 1.65 (4H, s), 1.38 (6H, s) |
| | 2-amine bis | | | |
| | acetic acid salt | | | |

### EXAMPLE 18

### Synthesis of 7,7-dimethyl-4-[3-(methylamino)pyrrolidin-1-yl]-5,6,7,8-tetrahydroquinazolin-2-amine (Compound 53)

Intermediate 17 (210 mg), tert-butyl methyl[pyrrolidin-3-yl]carbamate (CAS No 172478-00-1) (200 mg) and triethylamine (0.2 ml) are dissolved in NMP (2 ml) and heated under microwave irradiation at 150° C. for 30 mins. After cooling to room temperature, the mixture is diluted with H₂O and extracted with EtOAc. The organic layer is dried over MgSO₄, filtered and evaporated under reduced pressure. The crude reaction mixture is dissolved in DCM (10 ml) and TFA (5 ml) is added. The solution is stirred at room temperature for 1 hour and concentrated in vacuo (azeotroped with heptane). The residue is dissolved in H₂O (10 ml), washed with ether (5 ml) and the aqueous layer is basified with 15% NaOH. The aqueous layer is extracted with DCM (2×25 ml) and the organic layer dried over MgSO₄, filtered and evaporated in vacuo. The residue is purified by flash chromatography, eluting with DCM-MeOH—NH₄OH (92:7:1) to afford the title compound as a colourless solid (40 mg, 15%). R_{f} (DCM-MeOH 94:6) 0.29. LCMS 276 [M+H]⁺, RT 2.13 mins (pH 5.8). ¹H NMR 300 MHz (CDCl₃) (δ ppm): 4.45 (2H, br s), 3.80 (2H, m), 3.68 (1H, m), 3.47 (1H, m), 3.25 (1H, m), 2.68 (2H, m), 2.48 (3H, s), 2.35 (2H, s), 2.08 (1H, m), 1.73 (1H, m), 1.68 (1H, br s), 1.43 (2H, t), 1.00 (6H, s).

Compounds 54 and 55 are prepared in a similar manner to the method described for Compound 53 in Example 18. The reagents used and the results obtained are tabulated below (Table 23). The free base of the compounds is obtained unless otherwise stated.

TABLE 23

| | | | | ¹H NMR |
|---|---|---|---|---|
| Comp. | | Starting | | (Solvent, |
| No | IUPAC Name | Materials | LCMS | δ ppm) |
| 54 | 7,7-dimethyl-4- | Intermediate | 276 [M + H]⁺, | CDCl₃ 4.55 (2H, br s), 3.69 (2H, |
| | [(3S)-3- | 17, tert-butyl | RT 2.02 mins | m), 3.00 (2H, m), 2.92 (1H, m), |
| | methylpiperazin- | (2S)-2- | (pH | 2.80 (1H, m), 2.40-2.50 (3H, m), |
| | 1-yl]-5,6,7,8- | methylpiperazine- | 5.8) | 2.42 (2H, s), 1.60 (1H, br s), |
| | tetrahydroquinazolin- | 1- | | 1.45 (2H, t), 1.10 (3H, d), 1.03 (6H, s) |
| | 2-amine | carboxylate | | |
| | | (CAS RN | | |
| | | 169447-70-5) | | |
| 55 | 7,7-dimethyl-4- | Intermediate | 290 [M + H]⁺, | CDCl₃ 4.45 (2H, br s), 4.10 (1H, |
| | [(3S)-3-methyl- | 17, (S)-1-tert- | RT 1.87 mins | dd), 3.93 (1H, dt), 3.40 (1H, m), |
| | 1,4-diazepan-1- | butyloxycarbonyl- | (pH | 3.10 (2H, m), 2.82 (1H, dd), |
| | yl]-5,6,7,8- | 2-methyl- | 5.8) | 2.50 (2H, m), 2.39 (2H, s), 1.82 (2H, |
| | tetrahydroquinazolin- | [1,4]diazepane | | m), 1.65 (1H, br s), 1.50 (2H, m), |
| | 2-amine | | | 1.10 (3H, d), 1.00 (6H, s) |

Compounds 56 and 57 are prepared in a similar manner to the method described for Compound 2 in Example 12. The reagents used and the results obtained are tabulated below (Table 24). The free base of the compounds is obtained unless otherwise stated.

TABLE 24

| | | | | ¹H NMR |
|---|---|---|---|---|
| Comp. | | Starting | | (Solvent, |
| No | IUPAC Name | Materials | LCMS | δ ppm) |
| 56 | 8,8-dimethyl-4- | Intermediate | 276 [M + H]⁺, | CDCl₃ 5.60 (2H, bs), 3.85 (2H, |
| | (3- | 51 | RT 2.17 mins | m), 3.15 (1H, m), 3.05 (1H, m), |
| | methylpiperazin- | | (pH | 2.98 (2H, d), 2.68 (1H, m), |
| | 1-yl)-5,6,7,8- | | 5.8) | 2.41 (2H, bs), 1.65 (4H, s), 1.30 (6H, |
| | tetrahydroquinazolin- | | | s), 1.15 (3H, d) |
| | 2-amine | | | |
| 57 | 4-(3- | Intermediate | 248 [M + H]⁺, | CDCl₃ 4.60 (2H, bm), 4.00 (3H, |
| | aminoazetidin-1- | 52 | RT 1.51 mins | bm), 2.42 (2H, t), 1.70 (2H, m), |
| | yl)-8,8-dimethyl- | | (pH | 1.63 (2H, m), 1.38 (6H, s), |
| | 5,6,7,8- | | 5.8) | 1.23 (2H, s) |
| | tetrahydroquinazolin- | | | |
| | 2-amine | | | |

Compounds 58 and 59 are prepared in a similar manner to the method described for Intermediate 1 in Example 1. The reagents used and the results obtained are tabulated below (Table 25). The free base of the compounds is obtained unless otherwise stated.

TABLE 25

| | | | | ¹H NMR |
|---|---|---|---|---|
| Comp. | | Starting | | (Solvent, |
| No | IUPAC Name | Materials | LCMS | δ ppm) |
| 58 | 4-(1,4-diazepan- | Intermediate | 304 [M + H]⁺, | d₄-MeOH 3.70-3.98 (4H, mm), |
| | 1-yl)-7-isobutyl- | 43, | RT 2.12 mins | 2.69-2.81 (2H, mm), |
| | 5,6,7,8- | homopiperazine | (pH | 2.59-2.68 (2H, m), 1.74-2.26 (8H, mm), |
| | tetrahydroquinazolin- | | 5.8) | 1.16-1.32 (4H, mm), 0.96 (6H, |
| | 2-amine | | | d) |
| | acetic acid salt | | | |
| 59 | 7-isobutyl-4-(4- | Intermediate | 304 [M + H]⁺, | d₄-MeOH 3.43-3.54 (2H, m), |
| | methylpiperazin- | 43, piperazine | RT 2.67 mins | 3.18-3.33 (2H, m obscured by |
| | 1-yl)-5,6,7,8- | | (pH | MeOH peak), 2.65 (1H, dd), |
| | tetrahydroquinazolin- | | 5.8) | 2.37-2.58 (5H, mm), 2.26 (3H, s), |
| | 2-amine | | | 2.06 (1H, dd), 1.60-1.90 (3H, |
| | acetic acid salt | | | m), 0.98-1.22 (4H, mm), |
| | | | | 0.84 (6H, d) |

### EXAMPLE 19

### Synthesis of 7,7-dimethyl-4-[3-(methylamino)azetidin-1-yl]-5,6,7,8-tetrahydroquinazolin-2-amine (Compound 60)

tert-Butyl azetidin-3-ylcarbamate (300 mg) and Intermediate 17 (300 mg) are dissolved in NMP (3 ml) and triethylamine (0.5 ml) is added. The mixture is heated at 140° C. in a microwave reactor for 1 hour, then added to water and extracted with EtOAc (2×10 ml). The solvent is washed with water, dried and evaporated and the crude product dissolved in THF (10 ml). Lithium aluminium hydride (100 mg) is added and the solution heated at reflux for 18 hours. Water (0.1 ml), sodium hydroxide (15% aq, 0.1 ml) and water (0.3 ml) are added cautiously and the mixture stirred for 1 hour, then filtered and the filtrate evaporated in vacuo. The residue is purified by preparative HPLC (pH 5.8) to give the title compound as colourless solid (10 mg). LCMS 262 [M+H]⁺, RT 1.99 mins (pH 5.8). ¹H NMR 300 MHz (CDCl₃) (δ ppm): 5.15 (2H, br s), 4.40 (2H, dd), 3.93 (2H, dd), 3.62 (1H, m), 2.50 (2H, t), 2.43 (3H, s), 2.35 (2H, s), 1.48 (2H, t), 0.95 (6H, s).

Compounds 61 and 63 are prepared in a similar manner to the method described for Compound 2 in Example 12. The reagents used and the results obtained are tabulated below (Table 26). The free base of the compounds is obtained unless otherwise stated.

TABLE 26

| | | | | ¹H NMR |
|---|---|---|---|---|
| Comp. | | Starting | | (Solvent, |
| No | IUPAC Name | Materials | LCMS | δ ppm) |
| 61 | 8,8-dimethyl-4- | Intermediate | 302 [M + H]⁺, | CDCl₃ 4.65 (2H, bm), 3.78 (2H, |
| | [(4aR*,7aR*)- | 53 | RT 1.41 mins | m), 3.48 (2H, m), 3.30 (1H, m), |
| | octahydro-6H- | | (pH | 3.00 (1H, m), 2.65 (3H, m), |
| | pyrrolo[3,4- | | 5.8) | 2.21 (1H, m), 1.40-1.80 (6H, m), |
| | b]pyridin-6-yl]- | | | 1.28 (3H, s), 1.25 (3H, s), 0.90 (2H, m) |
| | 5,6,7,8- | | | |
| | tetrahydroquinazolin- | | | |
| | 2-amine | | | |
| 62 | 8,8-dimethyl-N- | Intermediate | 276 [M + H]⁺, | CDCl₃ 5.12 (2H, bs), 4.41 (1H, |
| | 4-piperidin-4-yl- | 54 | RT 1.68 mins | bd), 4.09 (1H, m), 3.11 (2H, m), |
| | 5,6,7,8- | | (pH | 2.69-2.87 (3H, m), 2.19 (2H, m), |
| | tetrahydroquinazoline- | | 5.8) | 2.05 (2H, m), 1.79 (2H, m), |
| | 2,4- | | | 1.62 (2H, m), 1.35 (2H, m), 1.25 (6H, |
| | diamine | | | s) |
| 63 | 8,8-dimethyl-N- | Intermediate | 262 [M + H]⁺, | CDCl₃ 5.90 (2H, bs), 5.41 (1H, |
| | 4-pyrrolidin-3-yl- | 55 | RT 1.81 mins | m), 4.64 (1H, m), 3.21-3.37 (3H, |
| | 5,6,7,8- | | (pH | m), 3.00-3.13 (2H, m), |
| | tetrahydroquinazoline- | | 5.8) | 2.15-2.36 (3H, m), 1.72-1.94 (3H, m), |
| | 2,4- | | | 1.62 (2H, m), 1.28 (6H, s) |
| | diamine | | | |

Compound 64 is prepared in a similar manner to the method described for Compound 53 in Example 18.

### Synthesis of 8,8-dimethyl-4-[(3S)-3-methylpiperazin-1-yl]-5,6,7,8-tetrahydroquinazolin-2-amine (Compound 64)

Starting from Intermediate 50 and tert-butyl (2S)-2-methylpiperazine-1-carboxylate. LCMS 276 [M+H]⁺, RT 2.02 mins (pH 5.8). ¹H NMR 300 MHz (CDCl₃) (δ ppm): 4.78 (2H, bs), 3.57-3.69 (2H, m), 2.68-3.10 (5H, m), 2.33-2.55 (3H, m), 1.58-1.73 (4H, m), 1.27 (6H, s), 1.10 (3H, d).

### EXAMPLE 20

### Synthesis of 6,6-dimethyl-4-(4-methylpiperazin-1-yl)-6,7-dihydro-5H-cyclopenta[d]pyrimidin-2-amine (Compound 65)

Methyl 4,4-dimethyl-2-oxocyclopentanecarboxylate (CAS No 60585-44-6) (1.138 g) and guanidine carbonate (1.205 g) are dissolved in EtOH (150 ml) and heated at 75° C. for 8.5 hours. The precipitate obtained is filtered and washed with EtOH. The filtrate is concentrated in vacuo to give an oil. This is partitioned between EtOAc (70 ml) and saturated brine (25 ml). The aqueous layer is reextracted with EtOAc (2×70 ml) and the combined organic layer is dried over MgSO₄, filtered and evaporated in vacuo to give an orange oil (1.003 g). The crude reaction mixture is suspended in a mixture of DCM (20 ml) and MeOH (20 ml), and 2M HCl in ether (2.8 ml) is added. The mixture is stirred at room temperature for 5 mins to give a clear solution. The excess solvent and HCl are removed in vacuo and azeotroped with heptane (2×10 ml). The residue obtained is resuspended in POCl₃ (13.5 ml) and the mixture is heated at 115° C. for 1 hour. The excess POCl₃ is removed under reduced pressure. After cooling to 0° C. (ice-bath), the reaction mixture is quenched with ice-water (40 ml). The solution is basified with NH₄OH (5 ml) to pH 9-10 and extracted with DCM (3×50 ml). The organic layer is dried (MgSO₄), filtered and evaporated in vacuo to give a brown oil (0.911 g). After purification by chromatography on silica using 3% MeOH in DCM, fractions containing material of R_{f}=0.15 (DCM-MeOH 97:3) are combined and evaporated under reduced pressure to give a red oil (94.1 mg). To this are added N-methylpiperazine (79.20, DIPEA (91.20 and NMP (1.32 ml) and the mixture is heated at 180° C. for 40 mins under microwave irradiation. The mixture is diluted with a 1:1 mixture of EtOAc and MTBE (120 ml) and washed with saturated brine (3×30 ml). The organic layer is dried (MgSO₄), filtered and removed in vacuo. Purification of the residual oil by flash chromatography, eluting with DCM-MeOH—NH₃ solution in MeOH (92:7:1) affords the title compound as a brown solid (10.2 mg, 0.6% overall yield). R_{f} 0.26, DCM-MeOH—NH₃ solution (92:7:1). LCMS 262 [M+H]⁺, RT 1.64 mins (pH 5.8). ¹H NMR 300 MHz (CDCl₃) (δ ppm): 4.60 (2H, bs), 3.60-3.70 (4H, m), 2.70 (2H, s), 2.55 (2H, s), 2.40-2.50 (4H, m), 2.30 (3H, s), 1.15 (6H, s).

Compound 66 is prepared in a similar manner to the method described for Intermediate 1 in Example 1.

### Synthesis of 4′-(4-methylpiperazin-1-yl)-5′,8′-dihydro-6′H-spiro[cyclohexane-1,7′-quinazolin]-2′-amine (Compound 66)

Starting from Intermediate 58 and N-methylpiperazine. LCMS 316 [M+H]⁺, RT 1.26 mins (pH 2.5). ¹H NMR 300 MHz (CDCl₃) (δ ppm): 5.27 (2H, bs), 3.40 (4H, m), 2.38-2.56 (8H, m), 2.34 (3H, s), 1.21-1.59 (12H, m).

### EXAMPLE 21

### Synthesis of 4-(4-methylpiperazin-1-yl)-7-[4-(trifluoromethyl)pyrimidin-2-yl]-5,6,7,8-tetrahydropyrido[3,4-d]pyrimidin-2-amine (Compound 67)

Intermediate 67 (68 mg) and 15% aqueous KOH (0.75 ml) are treated with 1,4-dioxane (1.5 ml) and heated under microwave irradiation at 140° C. for 30 mins. The solution is concentrated in vacuo and the residue extracted with EtOAc (2×10 ml), the extracts dried (MgSO₄) and concentrated in vacuo to afford the title compound as a near-colourless glass (55.6 mg, 99%). LCMS 395 [M+H]⁺, RT 2.64 mins (pH 5.8). ¹H NMR 300 MHz (CDCl₃) (δ ppm): 8.52 (1H, d), 6.79 (1H, d), 4.80 (2H, s), 4.75 (2H, s), 4.01 (2H, t), 3.40 (4H, m), 2.64 (2H, t), 2.50 (4H, m), 2.33 (3H, s).

Compound 68 is prepared in a similar manner to the method described for Compound 67 in Example 21.

### Synthesis of 6-[2-amino-4-(4-methylpiperazin-1-yl)-5,8-dihydropyrido[3,4-d]pyrimidin-7(6H)-yl]nicotinonitrile (Compound 68)

Starting from Intermediate 64. LCMS 351 [M+H]⁺, RT 2.19 mins (pH 5.8). ¹H NMR 300 MHz (CDCl₃) (δ ppm): 8.44 (1H, d), 7.66 (1H, dd), 6.60 (1H, d), 4.70 (2H, bs), 4.44 (2H, s), 3.95 (2H, m), 3.40 (4H, m), 2.65 (2H, m), 2.49 (4H, m), 2.33 (3H, s).

Compound 69 is prepared in a similar manner to the method described for Compound 2 in Example 12.

### Synthesis of 4′-[(4aR*,7aR*)-octahydro-6H-pyrrolo[3,4-b]pyridin-6-yl]-5′,8′-dihydro-6′H-spiro[cyclohexane-1,7′-quinazolin]-2′-amine (Compound 69)

Starting from Intermediate 68. LCMS 342 [M+H]⁺, RT 2.22 mins (pH 5.8). ¹H NMR 300 MHz (CDCl₃) (δ ppm): 5.08 (2H, bs), 3.71-3.90 (2H, m), 3.48-3.68 (2H, m), 3.32 (1H, m), 2.95-3.07 (1H, m), 2.18-2.69 (6H, m), 1.20-1.83 (16H, m), 0.84-0.97 (1H, m).

Compounds 70 to 72 are prepared in a similar manner to the method described for Intermediate 1 in Example 1. The reagents used and the results obtained are tabulated below (Table 27). The free base of the compounds is obtained unless otherwise stated.

TABLE 27

| | | | | ¹H NMR |
|---|---|---|---|---|
| Comp. | | Starting | | (Solvent, |
| No | IUPAC Name | Materials | LCMS | δ ppm) |
| 70 | 4′-[(3S)-3- | Intermediate | 316 [M + H]⁺, | CDCl₃ 6.80 (2H, bs), 4.00 (2H, d), |
| | methylpiperazin- | 58, (S)-(+)-2- | RT 2.35 mins | 2.90-3.25 (4H, m), |
| | 1-yl]-5′,8′- | methylpiperazine | (pH | 2.75-2.85 (1H, m), 2.50 (2H, s), |
| | dihydro-6′H- | (CAS RN | 5.8) | 2.35-2.45 (2H, m), 2.05 (6H, s, 2AcOH), |
| | spiro[cyclohexane- | 74879-18-8) | | 1.25-1.60 (13H, m), 1.22 (3H, d) |
| | 1,7′- | | | |
| | quinazolin]-2′- | | | |
| | amine bis acetic | | | |
| | acid salt | | | |
| 71 | 4′-(1,4-diazepan- | Intermediate | 316 [M + H]⁺, | CDCl₃ 7.20 (2H, bs), |
| | 1-yl)-5′,8′- | 58, | RT 2.37 mins | 3.70-3.85 (4H, m), 3.15 (2H, dd), 2.95 (2H, |
| | dihydro-6′H- | homopiperazine | (pH | dd), 2.50 (2H, s), 2.45 (2H, dd), |
| | spiro[cyclohexane- | | 5.8) | 2.05 (6H, s, 2AcOH), |
| | 1,7′- | | | 1.90-2.00 (2H, m), 1.20-1.55 (13H, m) |
| | quinazolin]-2′- | | | |
| | amine bis acetic | | | |
| | acid salt | | | |
| 72 | 4′-piperazin-1-yl- | Intermediate | 302 [M + H]⁺, | CDCl₃ 6.70 (2H, bs), |
| | 5′,8′-dihydro-6′H- | 58, piperazine | RT 2.38 mins | 3.50-3.60 (4H, m), 2.95-3.05 (4H, m), |
| | spiro[cyclohexane- | | (pH | 2.50 (2H, s), 2.40 (2H, dd), 2.05 (6H, s, |
| | 1,7′- | | 5.8) | 2AcOH), 1.20-1.55 (13H, m) |
| | quinazolin]-2′- | | | |
| | amine bis acetic | | | |
| | acid salt | | | |

Compounds 73 and 74 are prepared in a similar manner to the method described for Compound 2 in Example 12. The reagents used and the results obtained are tabulated below (Table 28). The free base of the compounds is obtained unless otherwise stated.

TABLE 28

| | | | | ¹H NMR |
|---|---|---|---|---|
| Comp. | | Starting | | (Solvent, |
| No | IUPAC Name | Materials | LCMS | δ ppm) |
| 73 | 4′-[3- | Intermediate | 302 [M + H]⁺, | CDCl₃ 4.94 (2H, br s), 4.38 (2H, |
| | (methylamino)azetidin- | 69 | RT 2.26 mins | t), 3.90 (2H, t), 3.60 (1H, m), |
| | 1-yl]-5′,8′- | | (pH | 2.40-2.50 (7H, m + s), |
| | dihydro-6′H- | | 5.8) | 1.20-1.60 (13H, m) |
| | spiro[cyclohexane- | | | |
| | 1,7′- | | | |
| | quinazolin]-2′- | | | |
| | amine bis | | | |
| | acetate salt | | | |
| 74 | 4-(3- | Intermediate | 290 [M + H]⁺, | d₄-MeOH 3.71-4.10 (3H, m), |
| | aminopyrrolidin- | 70 | RT 2.37 mins | 3.58-3.68 (2H, m), |
| | 1-yl)-7-isobutyl- | | (pH | 2.63-2.85 (3H, m), 2.12-2.29 (2H, m), |
| | 5,6,7,8- | | 5.8) | 1.71-2.01 (3H, mm), 1.18-1.35 (4H, |
| | tetrahydroquinazolin- | | | m), 0.96 (6H, d) |
| | 2-amine bis | | | |
| | acetate salt | | | |

Compounds 75 and 76 are prepared in a similar manner to the method described for Intermediate 1 in Example 1. The reagents used and the results obtained are tabulated below (Table 29). The free base of the compounds is obtained unless otherwise stated.

TABLE 29

| | | | | ¹H NMR |
|---|---|---|---|---|
| Comp. | | Starting | | (Solvent, |
| No | IUPAC Name | Materials | LCMS | δ ppm) |
| 75 | 7-isopropyl-4-(4- | Intermediate | 290 [M + H]⁺, | CDCl₃ 5.61 (2H, br s), 3.55 (2H, |
| | methylpiperazin- | 73, N- | RT 2.38 mins | m), 3.35 (2H, m), 2.40-2.60 (8H, |
| | 1-yl)-5,6,7,8- | methylpiperazine | (pH | m), 2.35 (3H, s), 1.93 (1H, m), |
| | tetrahydroquinazolin- | | 5.8) | 1.45-1.65 (2H, m), 1.15 (1H, m), |
| | 2-amine | | | 0.95 (6H, d) |
| 76 | 7-isopropyl-4- | Intermediate | 290 [M + H]⁺, | CDCl₃ 8.90 (2CH₃CO₂H, br s), |
| | [(3S)-3- | 73, (S)-(+)-2- | RT 2.35 mins | 6.65 (2H, br s), 4.06 (1H, m), |
| | methylpiperazin- | methylpiperazine | (pH | 3.89 (1H, m), 2.70-3.30 (6H, m), |
| | 1-yl]-5,6,7,8- | | 5.8) | 2.30-2.50 (3H, m), 2.03 (2CH₃CO₂H, |
| | tetrahydroquinazolin- | | | s), 1.95 (1H, m), 1.60 (2H, m), |
| | 2-amine bis | | | 1.24 (3H, d), 1.13 (1H, m), |
| | acetic acid salt | | | 0.95 (6H, app. t) |

Compounds 77 and 78 are prepared in a similar manner to the method described for Compound 2 in Example 12. The reagents used and the results obtained are tabulated below (Table 30). The free base of the compounds is obtained unless otherwise stated.

TABLE 30

| | | | | ¹H NMR |
|---|---|---|---|---|
| Comp. | | Starting | | (Solvent, |
| No | IUPAC Name | Materials | LCMS | δ ppm) |
| 77 | 7-isopropyl-4- | Intermediate | 316 [M + H]⁺, | CDCl₃ 3.50-4.00 (4H, m), |
| | [(4aR*,7aR*)- | 74 | RT 2.03 mins | 3.39 (1H, m), 3.05 (1H, m), |
| | octahydro-6H- | | (pH | 2.50-2.90 (4H, m), 2.25-2.50 (2H, m), |
| | pyrrolo[3,4- | | 5.8) | 1.95 (1H, m), 1.20-1.85 (9H, m), |
| | b]pyridin-6-yl]- | | | 1.10 (1H, m), 0.93 (6H, app. t) |
| | 5,6,7,8- | | | |
| | tetrahydroquinazolin- | | | |
| | 2-amine | | | |
| 78 | 7-isopropyl-4-[3- | Intermediate | 276 [M + H]⁺, | CDCl₃ 4.80 (2H, br s), 4.40 (1H, |
| | (methylamino)azetidin- | 75 | RT 2.21 mins | t), 4.28 (1H, t), 3.94 (1H, dd), |
| | 1-yl]- | | (pH | 3.70 (1H, dd), 3.60 (1H, m), 2.50 (2H, |
| | 5,6,7,8- | | 5.8) | m), 2.41 (3H, s), 2.30 (2H, m), |
| | tetrahydroquinazolin- | | | 1.90 (2H, m), 1.40-1.60 (3H, m), |
| | 2-amine | | | 0.90 (6H, m) |

### EXAMPLE 22

### Synthesis of 2,7,7-trimethyl-4-(4-methylpiperazin-1-yl)-5,6,7,8-tetrahydroquinazoline (Compound 79)

4,4-Dimethyl-2-oxo-cyclohexanecarboxylic acid methyl ester (CAS No. 32767-46-7) (410 mg), acetamidine hydrochloride (253 mg) and sodium ethoxide (455 mg) are treated with methanol (3 ml) and heated in a sealed vial at 100° C. for 3.5 hours. The solution is quenched with water (2 ml), treated with saturated (aq.) NH₄Cl (20 ml) to pH˜8, and extracted into DCM (8×10 ml). The combined organic phase is dried over MgSO₄ and concentrated in vacuo to give a brown solid (366 mg). LCMS 193 [M+H]⁺, RT 1.58 mins (pH 2.5). ¹H NMR 300 MHz (CDCl₃) (δ ppm): 13.2 (1H, br s), 2.53 (2H, t), 2.47 (3H, s) 2.40 (2H, s), 1.54 (2H, t), 0.99 (6H, s). The solid (366 mg), DMAP (12 mg) and phosphorous oxychloride (0.87 ml) are dissolved in 1,4-dioxane (2 ml) and heated in a sealed vial at 100° C. for 24 hours. The solution is concentrated in vacuo and the residue purified by flash chromatography, eluting with Heptane-EtOAc (0-100%) to afford a colourless oil (33 mg). LCMS 211 [M+H]⁺, RT 3.66 mins (pH 2.5). ¹H NMR 300 MHz (CDCl₃) (δ ppm): 2.75 (2H, t), 2.64 (3H, s), 2.63 (2H, s), 1.65 (2H, t), 1.00 (6H, s). The oil (33 mg) and N-methyl piperazine (0.087 ml) are dissolved in absolute EtOH (1 ml) and heated in a sealed vial at 100° C. for 3 days. The solution is concentrated in vacuo and the residue purified by preparative HPLC (pH5.8) to afford the title compound as a colourless glass (18 mg, 3% overall yield). LCMS 275 [M+H]⁺, RT 2.30 mins (pH 5.8). ¹H NMR 300 MHz (CDCl₃) (δ ppm): 3.41 (4H, m), 2.55 (8H, m), 2.53 (3H, s), 2.37 (3H, s), 1.53 (2H, t), 1.03 (6H, s).

Compound 80 is prepared in a similar manner to the method described for Compound 79 in Example 22.

### Synthesis of 7,7-dimethyl-4-(4-methylpiperazin-1-yl)-5,6,7,8-tetrahydroquinazoline (Compound 80)

Starting from 4,4-dimethyl-2-oxo-cyclohexanecarboxylic acid methyl ester and using formamidine acetic acid salt and sodium ethoxide in the first step, phosphorous oxychloride and DMAP in the second step and N-methyl piperazine in the last step. Following purification by preparative HPLC (pH 5.8) the title compound is obtained as a colourless glass (13 mg, 2.6% overall yield). LCMS 261 [M+H]⁺, RT 2.29 mins (pH 5.8). ¹H NMR 300 MHz (CDCl₃) (δ ppm): 8.57 (1H, s), 3.43 (4H, m), 2.5-2.6 (8H, m), 2.35 (3H, s), 1.54 (2H, t), 1.05 (6H, s).

### BIOLOGICAL EXAMPLES

### EXAMPLE 23

### Human H₄R³Histamine Binding Assay

Cf. The Journal of Pharmacology and Experimental Therapeutics 2001, 299(1); 121-130.

³Histamine dihydrochloride (Amersham) binding to the human H₄ receptor is determined using CHO-hH₄R membranes (350 ug/ml; Euroscreen), SPA beads (GE Healthcare; 15 mg/ml) and histamine (20 μM) in assay buffer [Tris HCl (50 mM), EDTA (5 mM, pH 7.4), 0.1% fatty acid free BSA]. The test compounds (0.5% DMSO final) are incubated with the assay mix in 96-well Optiplates (Perkin Elmer) for 15 mins at room temperature prior to addition of ³H-histamine solution (10 nM); the final assay volume is 200 μl per well. The plates are sealed and incubated for 16 h at room temperature prior to detection of membrane bound radioligand on Topcount (Perkin Elmer). Unless otherwise noted, all reagents are purchased from Sigma. Affinity (pKᵢ) measurements are determined by assessing the concentration of compound necessary to displace 50% of the specifically bound 3H-histamine.

The compounds of the invention are tested in this assay and their Kᵢ/EC₅₀ measurements are less than 2 μM.

Compound 16 is tested in this assay and gives a Kᵢ/EC₅₀ between 2 and 5 nM.

### EXAMPLE 24

### Human H₄ GTPγS³⁵ Assay

Cf. The Journal of Pharmacology and Experimental Therapeutics 2000, 296(3); 1058-1066.

GTPγS³⁵-(Amersham) binding is determined using CHO-hH₄R membranes (Euroscreen; 50 μg/ml), SPA beads (GE Healthcare; 10 mg/ml), GDP (15 μM) and saponin (30 μg/ml) in assay buffer [20 mM Hepes, 100 mM NaCl, 10 mM MgCl, 1 mM EDTA (pH 7.4), 0.1% BSA) in 96-well Optiplates (Perkin Elmer). Test compounds (0.5% DMSO final) are added and plates are incubated for 1 h at room temperature. GTPγS³⁵-(300 μM) is added (final assay volume 200 μl/well) and plates are incubated for a further 90 mins at room temperature prior to centrifugation of plates and detection using Topcount (Perkin Elmer). Unless noted, all reagents are purchased from Sigma. Affinity/efficacy measurements (pKᵢ/pEC₅₀) are determined by assessing the concentration of compound necessary to inhibit 50% of the functional response to a fixed concentration of histamine (GTPγS³⁵-binding), or the concentration of compound to cause a 50% increase in GTPγS³⁵-binding.

The compounds of the invention are tested in this assay and their Kᵢ/EC₅₀ measurements are less than 2 μM.

Compound 16 is tested in this assay and gives a Kᵢ/EC₅₀ between 9 and 12 nM.

1. A compound having formula I or pharmaceutically acceptable salts thereof or stereoisomeric forms thereof, and the geometrical isomers, enantiomers, diastereoisomers, and pharmaceutically acceptable salts thereof 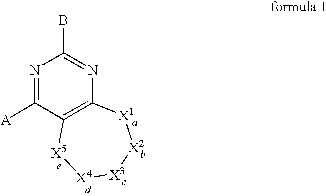
* represents the point of attachment to the rest of the molecule
wherein:
B is H, NH₂, cyclopropyl, C₁₋₃ alkyl optionally substituted by cyclopropyl, NRR′;
X¹ is C(R¹)(R²), O, S, SO₂, CO or NR³;
X² is C(R⁴)(R⁵), O, S, SO₂, CO or NR⁶;
X³ is C(R⁷)(R⁸), O, S, SO₂, CO or NR⁹;
X⁴ is C(R¹⁰)(R¹¹), O, S, SO₂, CO or NR¹²;
X⁵ is C(R¹³)(R¹⁴), O, S, SO₂, CO or NR¹⁵;
a is 0 or 1;
b is 0 or 1;
c is 0 or 1;
d is 0 or 1;
e is 0 or 1;
with the proviso that a+b+c+d+e=3 or 4 or 5;
R is H, C₁₋₃ alkyl;
R′ is C₁₋₃ alkyl;
R¹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;
R² is H; or is C₁₋₃ alkyl; or can form with R¹ a C₃₋₇ cycloalkyl which is spiro-fused to the cycle (formed by X¹-X⁵); or R² can form a methylene bridge with R⁵, R⁸, R¹¹ or R¹⁴;
R³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);
R⁴ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF2, OCF₃; or is heteroarylmethyloxy optionally substituted by C₁₋₃ alkyl, halogen, CF₃, C₁₋₃ alkoxy, OCHF₂, OCF₃;
R⁵ is H; or is C₁₋₃ alkyl; or can form with R⁴ a C₃₋₇ cycloalkyl which is spiro-fused to the cycle (formed by X¹-X⁵); or R⁵ can form a methylene bridge with R⁸, R¹¹ or R¹⁴;
R⁶ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);
R⁷ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;
R⁸ is H; or is C₁₋₃ alkyl; or can form with R⁷ a C₃₋₇ cycloalkyl which is spiro-fused to the cycle (formed by X¹-X⁵); or R⁸ can form a methylene bridge with R¹¹ or R¹⁴;
R⁹ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);
R¹⁰ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;
R¹¹ is H; or is C₁₋₃ alkyl; or can form with R¹⁰ a C₃₋₇ cycloalkyl which is spiro-fused to the cycle (formed by X¹-X⁵); or R¹¹ can form a methylene bridge with R¹⁴;
R¹² is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), —SO₂(heteroaryl); or is COO(C₁₋₄ alkyl);
R¹³ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, OCHF₂, OCF₃; or is aryl optionally substituted by C₁₋₃ alkyl, halogen, C₁₋₃ alkoxy, CF₃, OCHF₂, OCF₃; or is hydrogen; or is aryl C₁₋₂ alkyl; or is heteroaryl C₁₋₂ alkyl; or is C₁₋₆ alkyl optionally substituted with OH, OMe, F; or is C₃₋₆ cycloalkyl; or is C₂₋₆ alkenyl; or is C₅₋₇ cycloalkenyl; or is C₁₋₄ alkoxy optionally substituted by OH or OMe; or is C₁₋₄ alkoxymethyl; or is aryloxy optionally substituted by C₁₋₃ alkyl, halogen, CF₃, C₁₋₃ alkoxy, OCHF₂, OCF₃; or is heteroaryloxy optionally substituted by C₁₋₃ alkyl, halogen, CF₃, C₁₋₃ alkoxy, OCHF₂, OCF₃; or is benzyloxy optionally substituted by C₁₋₃ alkyl, halogen, CF₃, C₁₋₃ alkoxy, OCHF₂, OCF₃; or is heteroarylmethyloxy optionally substituted by C₁₋₃ alkyl, halogen, CF₃, C₁₋₃ alkoxy, OCHF₂, OCF₃;
R¹⁴ is H; or is C₁₋₃ alkyl; or can form with R¹³ a C₃₋₇ cycloalkyl which is spiro-fused to the cycle (formed by X¹-X⁵);
R¹⁵ is heterocycloalkyl optionally substituted by C₁₋₃ alkyl, halogen, C₁₋₃ alkoxy; or is heteroaryl optionally substituted by C₁₋₃ alkyl, halogen, C₁₋₃ alkoxy, CF₃, CN, OCHF₂, OCF₃; or is aryl optionally substituted by C₁₋₃ alkyl, halogen, C₁₋₃ alkoxy, CF₃, OCHF₂, OCF₃; or is C₁₋₆ alkyl; or is hydrogen; or is —COH, —CO(C₁₋₆ alkyl), —COaryl, —COheteroaryl, —SO₂(C₁₋₆ alkyl), —SO₂(aryl), SO₂(heteroaryl); or is COO(C₁₋₄ alkyl);
A is a group of formula II 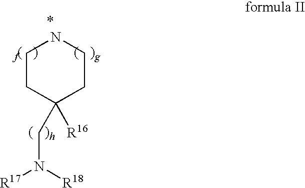
wherein
f is 0 or 1;
g is 0, 1 or 2;
h is 0 or 1;
R¹⁶ is hydrogen or unsubstituted C₁₋₃ alkyl;
R¹⁷ is hydrogen or unsubstituted C₁₋₃ alkyl;
R¹⁸ is hydrogen or unsubstituted C₁₋₃ alkyl;
or A is group of formula III 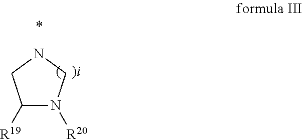
wherein
i is 2, 3;
R¹⁹ is hydrogen or unsubstituted C₁₋₃ alkyl;
R²⁰ is hydrogen or unsubstituted C₁₋₃ alkyl;
or A is a group of formula IV 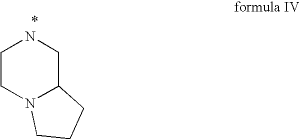
or A is a group of formula V 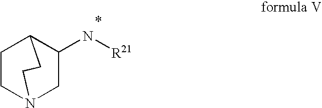
wherein
R²¹ is hydrogen or unsubstituted C₁₋₃ alkyl;
or A is a group of formula VI 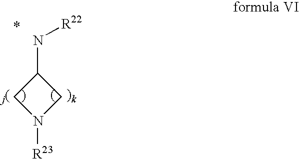
wherein
R²² is hydrogen or unsubstituted C₁₋₃ alkyl;
R²³ is hydrogen or unsubstituted C₁₋₃ alkyl;
j is 1 or 2;
k is 1 or 2;
or A is a group of formula VII 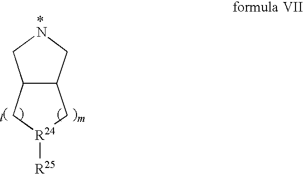
wherein
l is 1, 2 or 3;
m is 0, 1 or 2;
with the proviso that l+m=2 or 3;
R²⁴ is a CH group or N;
R²⁵ is hydrogen or unsubstituted C₁₋₃ alkyl group or is NH₂;
or A is a group of formula VIII 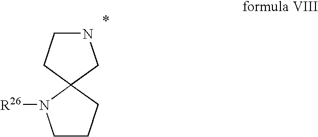
wherein
R²⁶ is hydrogen or is unsubstituted C₁₋₃ alkyl group;
or A is a group of formula IX 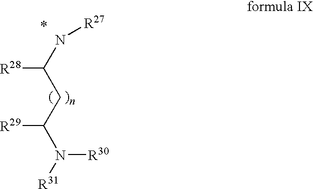
wherein
n is 0, 1 or 2;
R²⁷ is hydrogen or is unsubstituted C₁₋₃ alkyl group;
R²⁸ is hydrogen or is unsubstituted C₁₋₃ alkyl group;
R²⁹ is hydrogen or is unsubstituted C₁₋₃ alkyl group;
R³⁹ is hydrogen or is unsubstituted C₁₋₃ alkyl group;
R³¹ is hydrogen or is unsubstituted C₁₋₃ alkyl group.

2. The compound according to claim 1 wherein B is NH₂; and X¹ is C(R¹)(R²) or NR³; and X² is C(R⁴)(R⁵) or NR⁶; and X³ is C(R⁷)(R⁸) or NR⁹; and X⁴ is C(R¹⁰)(R¹¹) or NR¹²; and X⁵ is C(R¹³)(R¹⁴) or NR¹⁵.

3. The compound according to claim 1 wherein B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 0 or 1; and e is 0 or 1; and A is a group of formula II wherein f is 0 or 1; g is 0, 1 or 2; h is 0 or 1; R¹⁶ is hydrogen or unsubstituted C₁₋₃ alkyl; R¹⁷ is hydrogen or unsubstituted C₁₋₃ alkyl; R¹⁸ is hydrogen or unsubstituted C₁₋₃ alkyl.

4. The compound according to claim 1 wherein B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula III wherein i is 2, 3; R¹⁹ is hydrogen or unsubstituted C₁₋₃ alkyl; R²⁰ is hydrogen or unsubstituted C₁₋₃ alkyl.

5. The compound according to claim 1 wherein B is NH₂; and X¹ is C(R¹)(R²); and X² is NR⁶; and X³ is C(R⁷)(R⁸); and X⁴ is C(R¹⁰)(R¹¹); and a is 1; and b is 1; and c is 1; and d is 0 or 1; and e is 0; and A is a group of formula III wherein i is 2; R¹⁹ is hydrogen; R²⁰ is unsubstituted C₁₋₃ alkyl.

6. The compound according to claim 1 wherein B is CH₃ or H; and X¹ is C(R¹)(R²); and X² is C(R⁴)(R⁵); and X³ is C(R⁷)(R⁸); and X⁴ is C(R¹⁰)(R¹¹); and a is 1; and b is 1; and c is 1; and d is 1; and e is 0; and A is a group of formula III wherein i is 2; R¹⁹ is hydrogen; R²⁰ is unsubstituted C₁₋₃ alkyl.

7. The compound according to claim 1 wherein B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 0 or 1; and e is 0 or 1; and A is a group of formula VI wherein R²² is hydrogen or unsubstituted C₁₋₃ alkyl; R²³ is hydrogen or unsubstituted C₁₋₃ alkyl; j is 1 or 2, k is 2.

8. The compound according to claim 1 wherein B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula VII wherein l is 1, 2 or 3; m is 0, 1 or 2; R²⁴ is a CH group or N; R²⁵ is hydrogen or unsubstituted C₁₋₃ alkyl group or is NH₂; with the proviso that l+m=2 or 3.

9. The compound according to claim 1 wherein B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula VIII wherein R²⁶ is hydrogen or is unsubstituted C₁₋₃ alkyl group.

10. The compound according to claim 1 wherein B is NH₂; and X¹ is C(R¹)(R²); and X² is C(R⁴)(R⁵); and X³ is C(R⁷)(R⁸); and X⁴ is C(R¹⁰)(R¹¹); and X⁵ is C(R¹³)(R¹⁴); and a is 1; and b is 1; and c is 1; and d is 1; and e is 0 or 1; and A is a group of formula IX wherein n is 0; R²⁷ is hydrogen or is unsubstituted C₁₋₃ alkyl; R²⁸ is hydrogen or is unsubstituted C₁₋₃ alkyl; R²⁹ is hydrogen or is unsubstituted C₁₋₃ alkyl; R³⁹ is hydrogen or is unsubstituted C₁₋₃ alkyl; R³¹ is hydrogen or is unsubstituted C₁₋₃ alkyl.

11. The compound according to claim 1 selected from the group consisting of
4-(4-methylpiperazin-1-yl)-7-phenyl-5,6,7,8-tetrahydroquinazolin-2-amine;
4-(3-aminoazetidin-1-yl)-7-phenyl-5,6,7,8-tetrahydroquinazolin-2-amine;
4-(3-aminopyrrolidin-1-yl)-7-phenyl-5,6,7,8-tetrahydroquinazolin-2-amine;
4-[(4aR*,7aR*)-octahydro-6H-pyrrolo[3,4-b]pyridin-6-yl]-7-phenyl-5,6,7,8-tetrahydroquinazolin-2-amine;
4-(3-methylpiperazin-1-yl)-7-phenyl-5,6,7,8-tetrahydroquinazolin-2-amine;
7-(3-chlorophenyl)-4-(4-methylpiperazin-1-yl)-5,6,7,8-tetrahydroquinazolin-2-amine;
7-(3-chlorophenyl)-4-(1,4-diazepan-1-yl)-5,6,7,8-tetrahydroquinazolin-2-amine;
7-(3-chlorophenyl)-4-[(4aR*,7aR*)-octahydro-6H-pyrrolo[3,4-b]pyridin-6-yl]-5,6,7,8-tetrahydroquinazolin-2-amine;
7,7-dimethyl-4-(4-methylpiperazin-1-yl)-5,6,7,8-tetrahydroquinazolin-2-amine;
4-(1,4-diazepan-1-yl)-7,7-dimethyl-5,6,7,8-tetrahydroquinazolin-2-amine;
4-(3-aminopyrrolidin-1-yl)-7,7-dimethyl-5,6,7,8-tetrahydroquinazolin-2-amine;
7,7-dimethyl-4-[(4aR*,7aR*)-octahydro-6H-pyrrolo[3,4-b]pyridin-6-yl]-5,6,7,8-tetrahydroquinazolin-2-amine;
4-(3-aminopyrrolidin-1-yl)-7-(3-chlorophenyl)-5,6,7,8-tetrahydroquinazolin-2-amine;
7,7-dimethyl-4-(3-methylpiperazin-1-yl)-5,6,7,8-tetrahydroquinazolin-2-amine;
7,7-dimethyl-4-piperazin-1-yl-5,6,7,8-tetrahydroquinazolin-2-amine;
7-(2-chlorophenyl)-4-(4-methylpiperazin-1-yl)-5,6,7,8-tetrahydroquinazolin-2-amine;
7-(2-chlorophenyl)-4-(1,4-diazepan-1-yl)-5,6,7,8-tetrahydroquinazolin-2-amine;
4-(3-aminopyrrolidin-1-yl)-7-(2-chlorophenyl)-5,6,7,8-tetrahydroquinazolin-2-amine;
4-(1,4-diazepan-1-yl)-7-(4-fluorophenyl)-5,6,7,8-tetrahydroquinazolin-2-amine;
7-(4-fluorophenyl)-4-(4-methylpiperazin-1-yl)-5,6,7,8-tetrahydroquinazolin-2-amine;
4-piperazin-1-yl-7-pyridin-2-yl-5,6,7,8-tetrahydroquinazolin-2-amine;
4-(4-methylpiperazin-1-yl)-7-pyridin-2-yl-5,6,7,8-tetrahydroquinazolin-2-amine;
4-(1,4-diazepan-1-yl)-7-pyridin-2-yl-5,6,7,8-tetrahydroquinazolin-2-amine;
7-(5-chloro-2-thienyl)-4-piperazin-1-yl-5,6,7,8-tetrahydroquinazolin-2-amine;
5-(5-chloro-2-thienyl)-4-piperazin-1-yl-5,6,7,8-tetrahydroquinazolin-2-amine;
5-(5-chloro-2-thienyl)-4-(4-methylpiperazin-1-yl)-5,6,7,8-tetrahydroquinazolin-2-amine;
7-(5-chloro-2-thienyl)-4-(4-methylpiperazin-1-yl)-5,6,7,8-tetrahydroquinazolin-2-amine;
5-(5-chloro-2-thienyl)-4-(1,4-diazepan-1-yl)-5,6,7,8-tetrahydro quinazolin-2-amine;
7-(5-chloro-2-thienyl)-4-(1,4-diazepan-1-yl)-5,6,7,8-tetrahydroquinazolin-2-amine;
7-(4-fluorophenyl)-4-[(4aR*,7aR*)-octahydro-6H-pyrrolo[3,4-b]pyridin-6-yl]-5,6,7,8-tetrahydroquinazolin-2-amine trifluoroacetic acid salt;
7-(4-fluorophenyl)-N-4-[2-(methylamino)ethyl]-5,6,7,8-tetrahydroquinazoline-2,4-diamine;
7,7-dimethyl-N-4-[2-(methylamino)ethyl]-5,6,7,8-tetrahydroquinazoline-2,4-diamine;
4-(hexahydropyrrolo[3,4-c]pyrrol-2(1H)-yl)-7,7-dimethyl-5,6,7,8-tetrahydroquinazolin-2-amine;
7,7-dimethyl-4-[(3S)-3-methyl-1,4-diazepan-1-yl]-5,6,7,8-tetrahydroquinazolin-2-amine;
8,8-dimethyl-4-(3-methylpiperazin-1-yl)-5,6,7,8-tetrahydroquinazolin-2-amine;
4-(3-aminoazetidin-1-yl)-8,8-dimethyl-5,6,7,8-tetrahydroquinazolin-2-amine;
8,8-dimethyl-N-4-piperidin-4-yl-5,6,7,8-tetrahydroquinazoline-2,4-diamine;
8,8-dimethyl-N-4-pyrrolidin-3-yl-5,6,7,8-tetrahydroquinazoline-2,4-diamine;
8,8-dimethyl-4-[(3S)-3-methylpiperazin-1-yl]-5,6,7,8-tetrahydroquinazolin-2-amine;
6,6-dimethyl-4-(4-methylpiperazin-1-yl)-6,7-dihydro-5H-cyclopenta[d]pyrimidin-2-amine;
4-(4-methylpiperazin-1-yl)-7-[4-(trifluoromethyl)pyrimidin-2-yl]-5,6,7,8-tetrahydropyrido[3,4-d]pyrimidin-2-amine.

12. The compound according to claim 1 selected from the group consisting of
7,7-dimethyl-4-(4-methylpiperazin-1-yl)-5,6,7,8-tetrahydroquinazolin-2-amine;
4-(1,4-diazepan-1-yl)-7,7-dimethyl-5,6,7,8-tetrahydroquinazolin-2-amine;
4-(3-aminopyrrolidin-1-yl)-7,7-dimethyl-5,6,7,8-tetrahydroquinazolin-2-amine;
7,7-dimethyl-4-[(4aR*,7aR*)-octahydro-6H-pyrrolo[3,4-b]pyridin-6-yl]-5,6,7,8-tetrahydroquinazolin-2-amine;
7,7-dimethyl-4-(3-methylpiperazin-1-yl)-5,6,7,8-tetrahydroquinazolin-2-amine;
7,7-dimethyl-4-piperazin-1-yl-5,6,7,8-tetrahydroquinazolin-2-amine;
7-(5-chloro-2-thienyl)-4-piperazin-1-yl-5,6,7,8-tetrahydroquinazolin-2-amine;
7-(5-chloro-2-thienyl)-4-(1,4-diazepan-1-yl)-5,6,7,8-tetrahydroquinazolin-2-amine;
4-(3-aminoazetidin-1-yl)-7,7-dimethyl-5,6,7,8-tetrahydroquinazolin-2-amine;
7-isobutyl-4-piperazin-1-yl-5,6,7,8-tetrahydroquinazolin-2-amine;
8,8-dimethyl-4-(4-methylpiperazin-1-yl)-5,6,7,8-tetrahydroquinazolin-2-amine;
4-(1,4-diazepan-1-yl)-8,8-dimethyl-5,6,7,8-tetrahydroquinazolin-2-amine bis acetic acid salt;
7,7-dimethyl-4-[3-(methylamino)pyrrolidin-1-yl]-5,6,7,8-tetrahydroquinazolin-2-amine;
7,7-dimethyl-4-[(3S)-3-methylpiperazin-1-yl]-5,6,7,8-tetrahydroquinazolin-2-amine;
4-(1,4-diazepan-1-yl)-7-isobutyl-5,6,7,8-tetrahydroquinazolin-2-amine acetic acid salt;
7-isobutyl-4-(4-methylpiperazin-1-yl)-5,6,7,8-tetrahydroquinazolin-2-amine acetic acid salt;
7,7-dimethyl-4-[3-(methylamino)azetidin-1-yl]-5,6,7,8-tetrahydroquinazolin-2-amine;
8,8-dimethyl-4-[(4aR*,7aR*)-octahydro-6H-pyrrolo[3,4-b]pyridin-6-yl]-5,6,7,8-tetrahydro quinazolin-2-amine;
4′-(4-methylpiperazin-1-yl)-5′,8′-dihydro-6′H-spiro[cyclohexane-1,7-quinazolin]-2′-amine;
4′-[(4aR*,7aR*)-octahydro-6H-pyrrolo[3,4-b]pyridin-6-yl]-5′,8′-dihydro-6′H-spiro[cyclohexane-1,7′-quinazolin]-2-amine;
4′-[(3S)-3-methylpiperazin-1-yl]-5′,8′-dihydro-6′H-spiro[cyclohexane-1,7′-quinazolin]-2′-amine bis acetic acid salt;
4′-(1,4-diazepan-1-yl)-5′,8′-dihydro-6′H-spiro[cyclohexane-1,7′-quinazolin]-2′-amine bis acetic acid salt;
4′-piperazin-1-yl-5′,8′-dihydro-6′H-spiro[cyclohexane-1,7′-quinazolin]-2′-amine bis acetic acid salt;
4′-[3-(methylamino)azetidin-1-yl]-5′,8′-dihydro-6′H-spiro[cyclohexane-1,7′-quinazolin]-2′-amine bis acetate salt;
4-(3-aminopyrrolidin-1-yl)-7-isobutyl-5,6,7,8-tetrahydroquinazolin-2-amine bis acetate salt;
7-isopropyl-4-(4-methylpiperazin-1-yl)-5,6,7,8-tetrahydroquinazolin-2-amine;
7-isopropyl-4-[(3S)-3-methylpiperazin-1-yl]-5,6,7,8-tetrahydroquinazolin-2-amine bis acetic acid salt;
7-isopropyl-4-[(4aR*,7aR*)-octahydro-6H-pyrrolo[3,4-b]pyridin-6-yl]-5,6,7,8-tetrahydroquinazolin-2-amine;
7-isopropyl-4-[3-(methylamino)azetidin-1-yl]-5,6,7,8-tetrahydroquinazolin-2-amine.

13. A compound selected from the group consisting of
tert-butyl [1-(2-amino-6,7-dihydro-5H-cyclopenta[d]pyrimidin-4-yl)pyrrolidin-3-yl]carbamate;
tert-butyl [1-(2-amino-6,7,8,9-tetrahydro-5H-cyclohepta[d]pyrimidin-4-yl)pyrrolidin-3-yl]carbamate;
tert-butyl [1-(2-amino-6-phenyl-5,6,7,8-tetrahydroquinazolin-4-yl)pyrrolidin-3-yl]carbamate;
methyl 2-oxo-4-phenylcyclohexanecarboxylate;
2-amino-7-phenyl-5,6,7,8-tetrahydroquinazolin-4-ol;
4-chloro-7-phenyl-5,6,7,8-tetrahydroquinazolin-2-amine;
tert-butyl [1-(2-amino-7-phenyl-5,6,7,8-tetrahydroquinazolin-4-yl)azetidin-3-yl]carbamate;
tert-butyl [1-(2-amino-7-phenyl-5,6,7,8-tetrahydroquinazolin-4-yl)pyrrolidin-3-yl]carbamate;
tert-butyl (4aR*,7aR*)-6-(2-amino-7-phenyl-5,6,7,8-tetrahydroquinazolin-4-yl)octahydro-1H-pyrrolo[3,4-b]pyridine-1-carboxylate;
tert-butyl 4-(2-amino-7-phenyl-5,6,7,8-tetrahydroquinazolin-4-yl)-2-methylpiperazine-1-carboxylate;
4-(1-benzyl-1,7-diazaspiro[4.4]non-7-yl)-7-phenyl-5,6,7,8-tetrahydroquinazolin-2-amine;
methyl 4-(3-chlorophenyl)-2-oxocyclohexanecarboxylate;
2-amino-7-(3-chlorophenyl)-5,6,7,8-tetrahydroquinazolin-4-ol;
tert-butyl 4-[2-amino-7-(3-chlorophenyl)-5,6,7,8-tetrahydroquinazolin-4-yl]-1,4-diazepane-1-carboxylate;
tert-butyl (4aR*,7aR*)-6-[2-amino-7-(3-chlorophenyl)-5,6,7,8-tetrahydroquinazolin-4-yl]octahydro-1H-pyrrolo[3,4-b]pyridine-1-carboxylate;
2-amino-7,7-dimethyl-5,6,7,8-tetrahydroquinazolin-4-ol;
4-chloro-7,7-dimethyl-5,6,7,8-tetrahydroquinazolin-2-amine;
tert-butyl 4-(2-amino-7,7-dimethyl-5,6,7,8-tetrahydroquinazolin-4-yl)-1,4-diazepane-1-carboxylate;
tert-butyl [1-(2-amino-7,7-dimethyl-5,6,7,8-tetrahydroquinazolin-4-yl)pyrrolidin-3-yl]carbamate;
tert-butyl (4aR*,7aR*)-6-(2-amino-7,7-dimethyl-5,6,7,8-tetrahydroquinazolin-4-yl)octahydro-1H-pyrrolo[3,4-b]pyridine-1-carboxylate;
tert-butyl {1-[2-amino-7-(3-chlorophenyl)-5,6,7,8-tetrahydroquinazolin-4-yl]pyrrolidin-3-yl}carbamate;
tert-butyl 4-(2-amino-7,7-dimethyl-5,6,7,8-tetrahydroquinazolin-4-yl)-2-methylpiperazine-1-carboxylate;
methyl 4-(2-chlorophenyl)-2-oxocyclohexanecarboxylate;
2-amino-7-(2-chlorophenyl)-5,6,7,8-tetrahydroquinazolin-4-ol;
4-chloro-7-(2-chlorophenyl)-5,6,7,8-tetrahydroquinazolin-2-amine;
tert-butyl 4-[2-amino-7-(2-chlorophenyl)-5,6,7,8-tetrahydroquinazolin-4-yl]-1,4-diazepane-1-carboxylate;
tert-butyl (4aR*,7aR*)-6-[2-amino-7-(2-chlorophenyl)-5,6,7,8-tetrahydroquinazolin-4-yl] octahydro-1H-pyrrolo[3,4-b]pyri dine-1-carboxylate;
tert-butyl {1-[2-amino-7-(2-chlorophenyl)-5,6,7,8-tetrahydroquinazolin-4-yl]pyrrolidin-3-yl}carbamate;
2-amino-7-(4-fluorophenyl)-5,6,7,8-tetrahydroquinazolin-4-ol;
4-chloro-7-(4-fluorophenyl)-5,6,7,8-tetrahydroquinazolin-2-amine;
tert-butyl (4aR*,7aR*)-6-(2-amino-5,6,7,8-tetrahydroquinazolin-4-yl)octahydro-1H-pyrrolo[3,4-b]pyridine-1-carboxylate;
methyl 2-oxo-4-pyridin-2-ylcyclohexanecarboxylate;
2-amino-7-pyridin-2-yl-5,6,7,8-tetrahydroquinazolin-4-ol;
4-chloro-7-pyridin-2-yl-5,6,7,8-tetrahydroquinazolin-2-amine;
methyl 4-(5-chloro-2-thienyl)-2-oxocyclohexanecarboxylate;
methyl 2-(5-chloro-2-thienyl)-6-oxocyclohexanecarboxylate;
2-amino-7-(5-chloro-2-thienyl)-5,6,7,8-tetrahydroquinazolin-4-ol;
4-chloro-7-(5-chloro-2-thienyl)-5,6,7,8-tetrahydroquinazolin-2-amine;
4-chloro-5-(5-chloro-2-thienyl)-5,6,7,8-tetrahydroquinazolin-2-amine;
tert-butyl (4aR,7aR)-6-[2-amino-7-(4-fluorophenyl)-5,6,7,8-tetrahydroquinazolin-4-yl]octahydro-1H-pyrrolo[3,4-b]pyridine-1-carboxylate;
tert-butyl (2-{[2-amino-7-(4-fluorophenyl)-5,6,7,8-tetrahydroquinazolin-4-yl]amino}ethyl)methylcarbamate;
tert-butyl [1-(2-amino-7,7-dimethyl-5,6,7,8-tetrahydroquinazolin-4-yl)azetidin-3-yl]carbamate;
4-chloro-7-isobutyl-5,6,7,8-tetrahydroquinazolin-2-amine;
2-amino-6,6-dimethyl-5,6,7,8-tetrahydroquinazolin-4-ol;
4-chloro-6,6-dimethyl-5,6,7,8-tetrahydroquinazolin-2-amine;
tert-butyl {2-[(2-amino-7,7-dimethyl-5,6,7,8-tetrahydroquinazolin-4-yl)amino]ethyl}methylcarbamate;
tert-butyl 5-(2-amino-7,7-dimethyl-5,6,7,8-tetrahydroquinazolin-4-yl)hexahydropyrrolo[3,4-c]pyrrole-2(1H)-carboxylate;
tert-butyl 4-(2-amino-6,6-dimethyl-5,6,7,8-tetrahydroquinazolin-4-yl)-2-methylpiperazine-1-carboxylate;
2-amino-8,8-dimethyl-5,6,7,8-tetrahydroquinazolin-4-ol;
4-chloro-8,8-dimethyl-5,6,7,8-tetrahydroquinazolin-2-amine;
tert-butyl 4-(2-amino-8,8-dimethyl-5,6,7,8-tetrahydroquinazolin-4-yl)-2-methylpiperazine-1-carboxylate;
tert-butyl [1-(2-amino-8,8-dimethyl-5,6,7,8-tetrahydroquinazolin-4-yl)azetidin-3-yl]carbamate;
tert-butyl (4aR,7aR)-6-(2-amino-8,8-dimethyl-5,6,7,8-tetrahydroquinazolin-4-yl)octahydro-1H-pyrrolo[3,4-b]pyridine-1-carboxylate;
tert-butyl 4-[(2-amino-8,8-dimethyl-5,6,7,8-tetrahydroquinazolin-4-yl)amino]piperidine-1-carboxylate;
tert-butyl 3-[(2-amino-8,8-dimethyl-5,6,7,8-tetrahydroquinazolin-4-yl)amino]pyrrolidine-1-carboxylate;
methyl 2-oxospiro[5.5]undecane-3-carboxylate;
2′-amino-5′,8′-dihydro-6′H-spiro[cyclohexane-1,7′-quinazolin]-4′-ol;
4′-chloro-5′,8′-dihydro-6′H-spiro[cyclohexane-1,7′-quinazolin]-2′-amine;
tert-butyl 2-amino-4-hydroxy-5,8-dihydropyrido[3,4-d]pyrimidine-7(6H)-carboxylate;
tert-butyl 2-[(2,2-dimethylpropanoyl)amino]-4-hydroxy-5,8-dihydropyrido[3,4-d]pyrimidine-7(6H)-carboxylate;
N-(4-hydroxy-5,6,7,8-tetrahydropyrido[3,4-d]pyrimidin-2-yl)-2,2-dimethylpropanamide;
N-[7-(5-cyanopyridin-2-yl)-4-hydroxy-5,6,7,8-tetrahydropyrido[3,4-d]pyrimidin-2-yl]-2,2-dimethylpropanamide;
N-[4-chloro-7-(5-cyanopyridin-2-yl)-5,6,7,8-tetrahydropyrido[3,4-d]pyrimidin-2-yl]-2,2-dimethylpropanamide;
N-[7-(5-cyanopyridin-2-yl)-4-(4-methylpiperazin-1-yl)-5,6,7,8-tetrahydropyrido[3,4-d]pyrimidin-2-yl]-2,2-dimethylpropanamide;
N-{4-hydroxy-7-[4-(trifluoromethyl)pyrimidin-2-yl]-5,6,7,8-tetrahydropyrido[3,4-d]pyrimidin-2-yl}-2,2-dimethylpropanamide;
N-{4-chloro-7-[4-(trifluoromethyl)pyrimidin-2-yl]-5,6,7,8-tetrahydropyrido[3,4-d]pyrimidin-2-yl}-2,2-dimethylpropanamide;
2,2-dimethyl-N-{4-(4-methylpiperazin-1-yl)-7-[4-(trifluoromethyl)pyrimidin-2-yl]-5,6,7,8-tetrahydropyrido[3,4-d]pyrimidin-2-yl}propanamide;
tert-butyl (4aR,7aR)-6-(2′-amino-5′,8′-dihydro-6′H-spiro[cyclohexane-1,7′-quinazolin]-4′-yl)octahydro-1H-pyrrolo[3,4-b]pyridine-1-carboxylate;
tert-butyl [1-(2′-amino-5′,8′-dihydro-6′H-spiro[cyclohexane-1,7′-quinazolin]-4′-yl)azetidin-3-yl]methylcarbamate;
tert-butyl [1-(2-amino-7-isobutyl-5,6,7,8-tetrahydroquinazolin-4-yl)pyrrolidin-3-yl]carbamate;
methyl 4-isopropyl-2-oxocyclohexanecarboxylate;
2-amino-7-isopropyl-5,6,7,8-tetrahydroquinazolin-4-ol;
4-chloro-7-isopropyl-5,6,7,8-tetrahydroquinazolin-2-amine;
tert-butyl (4aR,7aR)-6-(2-amino-7-isopropyl-5,6,7,8-tetrahydroquinazolin-4-yl)octahydro-1H-pyrrolo[3,4-b]pyridine-1-carboxylate;
tert-butyl [1-(2-amino-7-isopropyl-5,6,7,8-tetrahydroquinazolin-4-yl)azetidin-3-yl]methylcarbamate.

14. A pharmaceutical composition comprising as active ingredient a therapeutically effective amount of a compound according to any claim 1 and a pharmaceutically acceptable adjuvant, diluent or carrier.

15. (canceled)

16. (canceled)

17. (canceled)

18. A method of treating a disease or disorder in a mammal, the method comprising administering to the mammal a therapeutically effective amount of a compound according to claim 1, wherein the disease or disorder is
a respiratory disease selected from adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, and allergic congestion;
a disorder of the genito-urinary tract selected from female and male sexual dysfunction, overactive bladder conditions, urinary incontinence, neurogenic detrusor overactivity, idiopathic detrusor overactivity, benign prostate hyperplasia and lower urinary tract symptoms;
a dermatological disease selected from dermatitis and psoriasis and itchy skin;
a disease of the cardiovascular system selected from thromboembolic diseases, atherosclerosis, myocardial infarction, angina pectoris, unstable angina, myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction;
a disease of the gastrointestinal tract selected from inflammatory bowel disease, Crohn's disease, ulcerative colitis;
an autoimmune diseases selected from rheumatoid arthritis, multiple sclerosis;
cancer;
pain; or
a lymphatic disease.

19. The method of claim 18 wherein the disease is
an inflammatory disorder,
a respiratory disease selected from adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic and congestion;
a dermatological disease selected from dermatitis and psoriasis and treatment of itchy skin or diseases of the gastrointestinal tract including inflammatory bowel disease, Crohn's disease, and ulcerative colitis; or
an autoimmune disease selected from rheumatoid arthritis, multiple sclerosis.
